(19) United States
(12) Patent Application Publication
(10) Pub. No.: US 2024/0047013 A1
(43) Pub. Date: Feb. 8, 2024

(54) SYSTEMS AND METHODS FOR SELECTING CELLS OF INTEREST BASED ON VISUALIZATION OF IMMUNE CELL DATA

(71) Applicant: 10X GENOMICS, INC., Pleasanton, CA (US)

(72) Inventors: Wyatt James MCDONNELL, Pleasanton, CA (US); David Benjamin JAFFE, Pleasanton, CA (US)

(73) Assignee: 10X GENOMICS, INC., Pleasanton, CA (US)

(21) Appl. No.: 18/353,001

(22) Filed: Jul. 14, 2023

Related U.S. Application Data
(63) Continuation of application No. PCT/US2022/013420, filed on Jan. 21, 2022.
(60) Provisional application No. 63/140,778, filed on Jan. 22, 2021.

(51) Int. Cl. G16B 45/00

(52) U.S. Cl. CPC G16B 45/00

(57) ABSTRACT

Methods and systems for selecting a cell of interest based on immune cell data are disclosed. For example, a method may comprise obtaining a single cell or spatial dataset, wherein the single cell or spatial dataset comprises a dataset of immune cell receptors, antibodies, or fragments thereof from a sample; identifying a clonotype group in the single cell or spatial dataset;

selecting a schema to visualize selected amino acids in the clonotype group based on positions or chemical identity of the selected amino acids; visualizing the selected amino acids in the clonotype group in a graphic representation according to the schema; and selecting a cell of interest from the clonotype group based on a pre-defined criterion using the graphic representation.

### CROSS-REFERENCE TO RELATED APPLICATIONS

This application claims priority to U.S. Provisional Patent Application No. 63/140,778, filed Jan. 22, 2021, which is incorporated by reference herein in its entirety.

### FIELD

This description is generally directed towards systems and methods for immune cell screening. More specifically, methods and systems are provided for screening immune cell datasets based on visualization of immune cell clonotype data.

### BACKGROUND

Current immune cell clonotype data analysis has difficulty with the selection of cells of interest from immune cell clonotype datasets due to problems such as inefficient and mentally taxing data display to render such display hard for interactive analysis. There is, therefore, a need for improved data analysis and visualization systems and methods to help interactive analysis and selection of cells of interest.

### SUMMARY

In accordance with various embodiments, methods and systems for selecting a cell of interest based on a single cell dataset arc disclosed. For example, a method may comprise obtaining a single cell dataset. For example, the single cell dataset comprises a dataset of immune cell receptors, antibodies, or fragments thereof from single cells. The method may further comprise identifying a clonotype group in the single cell dataset. The method may further comprise selecting a schema to visualize selected amino acids in the clonotype group based on positions or chemical identity of the selected amino acids. The method may further comprise visualizing the selected amino acids in the clonotype group in a graphic representation according to the schema. The method may further comprise selecting a cell of interest from the clonotype group based on a pre-defined criterion using the graphic representation.

In accordance with various embodiments, an interactive visualization system is disclosed. The system includes a data source for obtaining a single cell dataset, wherein the single cell dataset comprises a dataset of immune cell receptors, antibodies, or fragments thereof from single cells. The system includes one or more data processors. The system includes a computing device communicatively connected to the data source and configured to receive the single cell dataset. The computing device comprises a non-transitory computer readable storage medium containing instructions which, when executed on the one or more data processors, cause the one or more data processors to perform a method, the method comprising: identifying a clonotype group in the single cell dataset; selecting a schema to visualize selected amino acids in the clonotype group based on positions or chemical identity of the selected amino acids; and visualizing the selected amino acids in the clonotype group in a graphic representation according to the schema; and a display for rendering a visualization of the selected amino acids in the clonotype group in a graphic representation according to the schema.

In accordance with various embodiments, a method for producing an immunotherapeutic composition from cells selected from a single cell dataset may be provided. The method comprises obtaining a single cell dataset, wherein the single cell dataset comprises a dataset of immune cell receptors, antibodies, or fragments thereof from single cells. The method further comprises identifying a clonotype group in the single cell dataset. The method further comprises selecting a schema to visualize selected amino acids in the clonotype group based on positions or chemical identity of the selected amino acids. The method further comprises visualizing the selected amino acids in the clonotype group in a graphic representation according to the schema. The method further comprises selecting a cell of interest from the clonotype group based on a pre-defined criterion using the graphic representation. The method further comprises producing an immunotherapeutic composition using the cell of interest.

In accordance with various embodiments, a graphical user interface (GUI) for displaying immune cell clonotyping information is provided. The GUI can comprise a listing of exact subclonotypes of an immune cell clonotype, wherein the exact subclonotypes share identical V(D)J transcripts; a listing of one or more textual frames with information about chains common to each member of the immune cell clonotype, wherein the textual frame contains an amino acid sequence for variable and constant regions of each exact subclonotype; and positional information for selected amino acids of the amino acid sequence, wherein the selected amino acids are selected based on positions or chemical identity of the selected amino acids.

In accordance with various embodiments, a computer-program product tangibly embodied in a non-transitory machine-readable storage medium is provided. The computer-program product includes instructions configured to cause one or more data processors to perform a method for selecting a cell of interest based on a single cell dataset. The method comprises obtaining a single cell dataset, wherein the single cell dataset comprises a dataset of immune cell receptors, antibodies, or fragments thereof from single cells; identifying a clonotype group in the single cell dataset; selecting a schema to visualize selected amino acids in the clonotype group based on positions or chemical identity of the selected amino acids; visualizing the selected amino acids in the clonotype group in a graphic representation according to the schema; and selecting a cell of interest from the clonotype group based on a pre-defined criterion using the graphic representation.

In accordance with various embodiments, a method for selecting a cell of interest based on immune cell data is provided. The method includes obtaining the immune cell data, wherein the immune cell data comprises a dataset of immune cell receptors, antibodies, or fragments thereof from a sample. The method includes identifying a clonotype group in the immune cell data and selecting a schema to visualize selected amino acids in the clonotype group based on positions or chemical identity of the selected amino acids. The method includes visualizing the selected amino acids in the clonotype group in a graphic representation according to the schema. The method includes selecting a cell of interest from the clonotype group based on a pre-defined criterion using the graphic representation.

In accordance with various embodiments, an interactive visualization system is provided that comprises a data source for obtaining immune cell data, wherein the immune cell data comprises a dataset of immune cell receptors, antibodies, or fragments thereof from a sample; a computing device communicatively connected to the data source and configured to receive the immune cell data, the computing device comprising a set of processors and a non-transitory computer readable storage medium containing instructions, and a display for rendering a visualization of the selected amino acids in the clonotype group in the graphic representation according to the schema. The instructions, when executed by the set of processors, cause the set of processors to perform a method comprising: identifying a clonotype group in the immune cell data; selecting a schema to visualize selected amino acids in the clonotype group based on positions or chemical identity of the selected amino acids; and visualizing the selected amino acids in the clonotype group in a graphic representation according to the schema.

In accordance with various embodiments, a method for producing an immunotherapeutic composition from cells selected from immune cell data is provided. The method includes obtaining the immune cell data, wherein the immune cell data comprises a dataset of immune cell receptors, antibodies, or fragments thereof from a sample. The method includes identifying a clonotype group in the immune cell data. The method includes selecting a schema to visualize selected amino acids in the clonotype group based on positions or chemical identity of the selected amino acids. The method includes visualizing the selected amino acids in the clonotype group in a graphic representation according to the schema. The method includes selecting a cell of interest from the clonotype group based on a pre-defined criterion using the graphic representation; and producing an immunotherapeutic composition using the cell of interest.

In accordance with various embodiments, a computer-program product tangibly embodied in a non-transitory machine-readable storage medium is provided. The computer-program product includes instructions configured to cause one or more data processors to perform a method for selecting a cell of interest based on immune cell data. The method comprises obtaining the immune cell data, wherein the immune cell data comprises a dataset of immune cell receptors, antibodies, or fragments thereof from a sample; identifying a clonotype group in the immune cell data; selecting a schema to visualize selected amino acids in the clonotype group based on positions or chemical identity of the selected amino acids; visualizing the selected amino acids in the clonotype group in a graphic representation according to the schema; and selecting a cell of interest from the clonotype group based on a pre-defined criterion using the graphic representation.

These and other aspects and implementations are discussed in detail herein. The foregoing information and the following detailed description include illustrative examples of various aspects and implementations and provide an overview or framework for understanding the nature and character of the claimed aspects and implementations. The drawings provide illustration and a further understanding of the various aspects and implementations and are incorporated in and constitute a part of this specification.

### BRIEF DESCRIPTION OF FIGURES

The accompanying drawings are not intended to be drawn to scale. Like reference numbers and designations in the various drawings indicate like elements. For purposes of clarity, not every component may be labeled in every drawing. In the drawings:

FIG. 1 is a schematic illustration of non-limiting examples of a single cell sequencing workflow, in accordance with various embodiments.

FIG. 2 is a schematic illustration of non-limiting examples of a workflow for analyzing and visualizing immune cell clonotyping information, in accordance with various embodiments.

FIG. 3 is an example visualization displaying immune cell clonotyping information, in accordance with various embodiments.

FIG. 4 is an example visualization displaying immune cell clonotyping information, in accordance with various embodiments.

FIG. 5 is an example visualization displaying immune cell clonotyping information, in accordance with various embodiments.

FIG. 6 is an example visualization displaying immune cell clonotyping information, in accordance with various embodiments.

FIG. 7 is an example visualization displaying immune cell clonotyping information, in accordance with various embodiments.

FIG. 8 is an example visualization displaying immune cell clonotyping information, in accordance with various embodiments.

FIG. 9 is an example flow chart illustrating a method for displaying immune cell clonotyping information, in accordance with various embodiments.

FIG. 10 is an example flow chart illustrating a method for producing an immunotherapeutic composition from cells selected from a single cell dataset, in accordance with various embodiments.

FIG. 11 illustrates an interactive visualization system, in accordance with various embodiments.

FIG. 12 illustrates is a block diagram of a computer system, in accordance with various embodiments.

FIG. 13 is a schematic diagram showing an exemplary capture probe, in accordance with various embodiments.

FIG. 14 is a schematic illustrating a cleavable capture probe, wherein the cleaved capture probe can enter into a non-permeabilized cell and bind to analytes within the sample, in accordance with various embodiments.

FIG. 15 is a schematic diagram of an exemplary multiplexed spatially-barcoded feature, in accordance with various embodiments.

FIG. 16A is a schematic diagram illustrating an exemplary embodiment of a spatial methodology for generating immune cell data (e.g., sequence data for an antigen binding molecule (ABM), in accordance with various embodiments.

FIG. 16B is a schematic diagram illustrating an exemplary embodiment of a spatial methodology for generating immune cell data, in accordance with various embodiments.

FIG. 17 is a schematic diagram illustrating an exemplary analyte enrichment strategy following analyte capture on the array, in accordance with various embodiments.

FIG. 18 is a schematic diagram illustrating a sequencing strategy with a primer specific complementary to the sequencing flow cell attachment sequence (e.g., P5) and a custom sequencing primer complementary to a portion of the constant region of the analyte, in accordance with various embodiments.

FIG. 19 is a schematic diagram illustrating an exemplary nucleic acid library preparation method to remove a portion of an analyte sequence via double circularization of a member of a nucleic acid library, in accordance with various embodiments.

FIG. 20 is a schematic diagram illustrating another exemplary workflow for processing such double-stranded circularized nucleic acid product, in accordance with various embodiments.

FIG. 21 is a schematic diagram illustrating an exemplary nucleic acid library preparation method to remove all or a portion of a constant sequence of an analyte from a member of a nucleic acid library via circularization, in accordance with various embodiments.

FIG. 22 is a schematic diagram illustrating an exemplary nucleic acid library method to reverse the orientation of an analyte sequence in a member of a nucleic acid library, in accordance with various embodiments.

It is to be understood that the figures are not necessarily drawn to scale, nor are the objects in the figures necessarily drawn to scale in relationship to one another. The figures are depictions that are intended to bring clarity and understanding to various embodiments of apparatuses, systems, and methods disclosed herein. Wherever possible, the same reference numbers will be used throughout the drawings to refer to the same or like parts. Moreover, it should be appreciated that the drawings are not intended to limit the scope of the present teachings in any way.

### DETAILED DESCRIPTION

The following description of various embodiments is exemplary and explanatory only and is not to be construed as limiting or restrictive in any way. Other embodiments, features, objects, and advantages of the present teachings will be apparent from the description and accompanying drawings, and from the claims.

It should be understood that any use of subheadings herein is for organizational purposes and should not be read to limit the application of those subheaded features to the various embodiments herein. Each and every feature described herein is applicable and usable in all the various embodiments discussed herein and that all features described herein can be used in any contemplated combination, regardless of the specific example embodiments that arc described herein. It should further be noted that exemplary description of specific features are used, largely for informational purposes, and not in any way to limit the design, subfeature, and functionality of the specifically described feature.

### I. Exemplary Definitions and Context

Unless defined otherwise, all technical and scientific terms used herein have the same meaning as commonly understood by one of ordinary skill in the art to which their various embodiments belong.

All publications mentioned herein are incorporated herein by reference for the purpose of describing and disclosing devices, compositions, formulations, and methodologies which are described in the publication and which might be used in connection with the present disclosure.

As used herein, the terms “comprise”, “comprises”, “comprising”, “contain”, “contains”, “containing”, “have”, “having” “include”, “includes”, and “including” and their variants are not intended to be limiting, are inclusive or open-ended and do not exclude additional, unrecited additives, components, integers, elements or method steps. For example, a process, method, system, composition, kit, or apparatus that comprises a list of features is not necessarily limited only to those features but may include other features not expressly listed or inherent to such process, method, system, composition, kit, or apparatus.

Unless otherwise defined, scientific and technical terms used in connection with the present teachings described herein shall have the meanings that are commonly understood by those of ordinary skill in the art. Further, unless otherwise required by context, singular terms shall include pluralities and plural terms shall include the singular. Generally, nomenclatures utilized in connection with, and techniques of, cell and tissue culture, molecular biology, and protein and oligo- or polynucleotide chemistry and hybridization described herein are those well-known and commonly used in the art. Standard techniques are used, for example, for nucleic acid purification and preparation, chemical analysis, recombinant nucleic acid, and oligonucleotide synthesis. Enzymatic reactions and purification techniques are performed according to manufacturer's specifications or as commonly accomplished in the art or as described herein. The techniques and procedures described herein arc generally performed according to conventional methods well known in the art and as described in various general and more specific references that are cited and discussed throughout the instant specification. See, e.g., Sambrook et al., Molecular Cloning: A Laboratory Manual (Third ed., Cold Spring Harbor Laboratory Press, Cold Spring Harbor, N.Y. 2000). The nomenclatures utilized in connection with, and the laboratory procedures and techniques described herein are those well-known and commonly used in the art.

Where values are described as ranges, it will be understood that such disclosure includes the disclosure of all possible sub-ranges within such ranges, as well as specific numerical values that fall within such ranges irrespective of whether a specific numerical value or specific subrange is expressly stated.

The terms “a,” “an,” and “the,” as used herein, generally refers to singular and plural references unless the context clearly dictates otherwise. “A and/or B” is used herein to include all of the following alternatives: “A”, “B”, “A or B”, and “A and B”.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number. If the degree of approximation is not otherwise clear from the context, “about” means either within plus or minus 10% of the provided value, or rounded to the nearest significant figure, in all cases inclusive of the provided value. In some embodiments, the term “about” indicates the designated value ±up to 10%, up to ±5%, or up to ±1%.

The term “CDR3 (Complementarity-Determining Region 3), as used herein, refers to three complementarity-determining regions are the portions of the amino acid sequence of a T or B cell receptor which are predicted to bind to an antigen. The nucleotide region encoding CDR3 spans the V(D)J junction, making it more diverse than that of the other CDRs. This serves as a useful way to identify unique chains.

The term “barcode” may refer to a label, or identifier, that conveys or is capable of conveying information (e.g., information about an analyte in a sample, a bead, a feature, a capture probe, and/or a nucleic acid barcode molecule). A barcode can be part of an analyte, a capture probe, a reporter oligonucleotide, an analyte capture agent, or nucleic acid barcode molecule, or independent of an analyte, a capture probe, a reporter oligonucleotide, an analyte capture agent, or nucleic acid barcode molecule. A barcode can be attached to an analyte, a capture probe, a reporter oligonucleotide, an analyte capture agent, or nucleic acid barcode molecule in a reversible or irreversible manner. A particular barcode can be unique relative to other barcodes. Barcodes can have a variety of different formats. For example, barcodes can include polynucleotide barcodes, random nucleic acid and/or amino acid sequences, and synthetic nucleic acid and/or amino acid sequences. A barcode can be attached to an analyte or to another moiety or structure in a reversible or irreversible manner. A barcode can be added to, for example, a fragment of a deoxyribonucleic acid (DNA) or ribonucleic acid (RNA) sample before or during sequencing of the sample. Barcodes can allow for or facilitates identification and/or quantification of individual sequencing-reads. In some embodiments, a barcode can be configured for use as a fluorescent barcode. For example, in some embodiments, a barcode can be configured for hybridization to fluorescently labeled oligonucleotide probes. Barcodes can be configured to spatially resolve molecular components found in biological samples, for example, at single-cell resolution (e.g., a barcode can be or can include a “spatial barcode”). In some embodiments, a barcode includes two or more sub-barcodes that together function as a single barcode. For example, a polynucleotide barcode can include two or more polynucleotide sequences (e.g., sub-barcodes). In some embodiments, the two or more sub-barcodes are separated by one or more non-barcode sequences. In some embodiments, the two or more sub-barcodes are not separated by non-barcode sequences.

In some embodiments, a barcode can include one or more unique molecular identifiers (UMIs). Generally, a unique molecular identifier is a contiguous nucleic acid segment or two or more non-contiguous nucleic acid segments that function as a label or identifier for a particular analyte, or for a nucleic acid barcode molecule that binds a particular analyte (e.g., mRNA) via the capture sequence.

The term “barcoded nucleic acid molecule” generally refers to a nucleic acid molecule that results from, for example, the processing of a nucleic acid barcode molecule (e.g., a capture probe comprising a spatial barcode sequence) with a nucleic acid sequence (e.g., nucleic acid sequence complementary to a nucleic acid primer sequence encompassed by the nucleic acid barcode molecule). The nucleic acid sequence may be a targeted sequence or a non-targeted sequence. For example, hybridization and reverse transcription of a nucleic acid molecule (e.g., a messenger RNA (mRNA) molecule) of a cell with a nucleic acid barcode molecule (e.g., a nucleic acid barcode molecule containing a barcode sequence and a nucleic acid primer sequence complementary to a nucleic acid sequence of the mRNA molecule) results in a barcoded nucleic acid molecule that has a sequence corresponding to the nucleic acid sequence of the mRNA and the barcode sequence (or a reverse complement thereof). A barcoded nucleic acid molecule may serve as a template, such as a template polynucleotide, that can be further processed (e.g., amplified) and sequenced to obtain the target nucleic acid sequence. For example, a barcoded nucleic acid molecule may be further processed (e.g., amplified) and sequenced to obtain the nucleic acid sequence of the mRNA.

In some embodiments, where nucleic acid barcode molecule comprises a single cell barcode sequence, the nucleic acid barcode molecule may be hybridized to an analyte (e.g., a messenger RNA (mRNA) molecule) of a cell. Reverse transcription can generate a barcoded nucleic acid molecule that has a sequence corresponding to the nucleic acid sequence of the mRNA and the barcode sequence (or a reverse complement thereof). The processing of the nucleic acid molecule comprising the nucleic acid sequence, the nucleic acid barcode molecule, or both, can include a nucleic acid reaction, such as, in non-limiting examples, reverse transcription, nucleic acid extension, ligation, etc. For example, the nucleic acid molecule comprising the nucleic acid sequence may he subjected to reverse transcription and then be attached to the nucleic acid barcode molecule to generate the barcoded nucleic acid molecule, or the nucleic acid molecule comprising the nucleic acid sequence may be attached to the nucleic acid barcode molecule and subjected to a nucleic acid reaction (e.g., extension, ligation) to generate the barcoded nucleic acid molecule. The barcoded nucleic acid molecule may serve as a template, such as a template polynucleotide, that can be further processed (e.g., amplified) and sequenced to obtain the target nucleic acid sequence. For example, the barcoded nucleic acid molecule may be further processed (e.g., amplified) and sequenced to obtain the nucleic acid sequence of the nucleic acid molecule (e.g., mRNA).

The term “cell barcode,” as used herein, refers to a known nucleotide sequence that serves as a unique identifier for a single GEM droplet. Each barcode usually contains reads from a single cell.

The term “clonotype,” as used herein, refers to a set of adaptive immune cells that are clonal progeny of a fully recombined, unmutated common ancestor. T cell clonotypes are generally distinguished by the nucleotide sequence of the rearranged TCR, which does not undergo somatic hypermutation (SHM) in the majority of vertebrate species. B cell clonotypes are commonly divergent from each other at the nucleotide level. For this reason, B cell clonotypes also frequently contain multiple exact subclonotypes.

The term “exact subclonotype,” as used herein, refers to a subset of cells within a clonotype that share identical immune receptor sequences at the nucleotide level, spanning the entirety of the V, D, and J genes and the V(D)J junction. Exact subclonotypes share the same V, D, J, and C gene annotations (e.g., cells that have identical V(D)J sequences but different C genes or isotypes are split into distinct exact subclonotypes).

The term “sample,” as used herein, generally refers to a biological sample of a subject. The sample may be a tissue sample, such as a biopsy, core biopsy, needle aspirate, or fine needle aspirate. The sample may be a fluid sample, such as a blood sample, urine sample, or saliva sample. The sample may be a skin sample. The sample may be a cheek swap. The sample may be a plasma or serum sample. The sample may be a cell-free or cell free sample. A cell-free sample may include extracellular polynucleotides. Extracellular polynucleotides may be isolated from a bodily sample that may be selected from a group consisting of blood, plasma, serum, urine, saliva, mucosal excretions, sputum, stool and tears.

The term “subject,” as used herein, generally refers to an animal, such as a mammal (e.g., human) or avian (e.g., bird), or other organism, such as a plant. For example, the subject can be a vertebrate, a mammal, a rodent (e.g., a mouse), a primate, a simian or a human. Animals may include, but are not limited to, farm animals, sport animals, and pets. A subject can be a healthy or asymptomatic individual, an individual that has or is suspected of having a disease (e.g., cancer) or a pre-disposition to the disease, and/or an individual that is in need of therapy or suspected of needing therapy. A subject can be a patient. A subject can be a microorganism or microbe (e.g., bacteria, fungi, archaea, viruses). The term “non-human animals” includes all vertebrates, e.g., mammals, e.g., rodents, e.g., mice, nonhuman primates, and other mammals, such as e.g., sheep, dogs, cows, chickens, and non-mammals, such as amphibians, reptiles, etc.

The term “primer,” as used herein generally refers to a strand of RNA or DNA that serves as a starting point for nucleic acid (e.g., DNA) synthesis. A primer may be used in a primer extension reaction, which may be a nucleic acid amplification reaction, such as, for example, polymerase chain reaction (PCR) or reverse transcription PCR (RT-PCR). The primer may have a sequence that is capable of coupling to a nucleic acid molecule. Such sequence may be complementary to the nucleic acid molecule, such as a poly-T sequence or a predetermined sequence, or a sequence that is otherwise capable of coupling (e.g., hybridizing) to the nucleic acid molecule, such as a universal primer.

As used herein, the term “cell” is used interchangeably with the term “biological cell.” Non-limiting examples of biological cells include eukaryotic cells, plant cells, animal cells, such as mammalian cells, reptilian cells, avian cells, fish cells or the like, prokaryotic cells, bacterial cells, fungal cells, protozoan cells, or the like, cells dissociated from a tissue, such as muscle, cartilage, fat, skin, liver, lung, neural tissue, and the like, immunological cells, such as T cells, B cells, natural killer cells, macrophages, and the like, embryos (e.g., zygotes), oocytes, ova, sperm cells, hybridomas, cultured cells, cells from a cell line, cancer cells, infected cells, transfected and/or transformed cells, reporter cells and the like. A mammalian cell can be, for example, from a human, mouse, rat, horse, goat, sheep, cow, primate or the like.

As used herein, a genome is the genetic material of a cell or organism, including animals, such as mammals, e.g., humans. In humans, the genome includes the total DNA, such as, for example, genes, noncoding DNA and mitochondrial DNA. The human genome typically contains 23 pairs of linear chromosomes: 22 pairs of autosomal chromosomes plus the sex-determining X and Y chromosomes. The 23 pairs of chromosomes include one copy from each parent. The DNA that makes up the chromosomes is referred to as chromosomal DNA and is present in the nucleus of human cells (nuclear DNA). Mitochondrial DNA is located in mitochondria as a circular chromosome, is inherited from only the female patient, and is often referred to as the mitochondrial genome as compared to the nuclear genome of DNA located in the nucleus.

The phrase “sequencing” refers to any technique known in the art that allows the identification of consecutive nucleotides of at least part of a nucleic acid. Non-limiting exemplary sequencing techniques include RNA-seq (also known as whole transcriptome sequencing), Illumina™ sequencing, direct sequencing, random shotgun sequencing, Sanger dideoxy termination sequencing, whole-genome sequencing, massively parallel signature sequencing (VIPSS), sequencing by hybridization, pyrosequencing, capillary electrophoresis, gel electrophoresis, duplex sequencing, cycle sequencing, single-base extension sequencing, solid-phase sequencing, high-throughput sequencing, massively parallel signature sequencing, emulsion PCR, sequencing by reversible dye terminator, paired-end sequencing, near-term sequencing, exonuclease sequencing, sequencing by ligation, short-read sequencing, single-molecule sequencing, sequencing-by-synthesis, real-time sequencing, reverse-terminator sequencing, nanopore sequencing, 454 sequencing, Solexa Genome Analyzer sequencing, SOLiD™ sequencing, MS-PET sequencing, mass spectrometry, and any combination thereof

DNA (deoxyribonucleic acid) is a chain of nucleotides consisting of 4 types of nucleotides; A (adenine), T (thymine), C (cytosine), and G (guanine), and that RNA (ribonucleic acid) is comprised of 4 types of nucleotides; A, U (uracil), G, and C. Certain pairs of nucleotides specifically bind to one another in a complementary fashion (called complementary base pairing). That is, adenine (A) pairs with thymine (T) (in the case of RNA, however, adenine (A) pairs with uracil (U)), and cytosine (C) pairs with guanine (G). When a first nucleic acid strand binds to a second nucleic acid strand made up of nucleotides that are complementary to those in the first strand, the two strands bind to form a double strand. As used herein, “nucleic acid sequencing data,” “nucleic acid sequencing information,” “nucleic acid sequence,” “genomic sequence,” “genetic sequence,” or “fragment sequence,” or “nucleic acid sequencing read” denotes any information or data that is indicative of the order of the nucleotide bases (e.g., adenine, guanine, cytosine, and thymine/uracil) in a molecule (e.g., whole genome, whole transcriptome, exome, oligonucleotide, polynucleotide, fragment, etc.) of DNA or RNA. It should be understood that the present teachings contemplate sequence information obtained using all available varieties of techniques, platforms, or technologies, including, but not limited to: capillary electrophoresis, microarrays, ligation-based systems, polymerase-based systems, hybridization-based systems, direct or indirect nucleotide identification systems, pyrosequencing, ion-or pH-based detection systems, electronical-based systems, etc.

A “polynucleotide”, “nucleic acid”, or “oligonucleotide” refers to a linear polymer of nucleosides (including deoxyribonucleosides, ribonucleosides, or analogs thereof) joined by internucleosidic linkages. Typically, a polynucleotide comprises at least three nucleosides. Usually oligonucleotides range in size from a few monomeric units, e.g. 3-4, to several hundreds of monomeric units. Whenever a polynucleotide such as an oligonucleotide is represented by a sequence of letters, such as “ATGCCTG,” it will be understood that the nucleotides are in 5′- >3′ order from left to right and that “A” denotes deoxyadenosine, “C” denotes deoxycytidine, “G” denotes deoxyguanosine, and “T” denotes thymidine, unless otherwise noted. The letters A, C, G, and T may be used to refer to the bases themselves, to nucleosides, or to nucleotides comprising the bases, as is standard in the art.

The methods and systems described herein accomplish sequencing of nucleic acid molecules including, but not limited to, DNA (e.g., genomic DNA), RNA (e.g., mRNA, including full-length mRNA transcripts, and small RNAs, such as miRNA, tRNA, and rRNA), and cDNA. In various embodiments, the methods and systems described herein accomplish genomic sequencing of nucleic acid molecules (e.g., DNA, RNA, and mRNA). In various embodiments, the methods and systems described herein accomplish genomic sequencing of immune cell receptor sequences (e.g., DNA, RNA, and mRNA). In various embodiments, the methods and systems described herein can accomplish transcriptome sequencing, e.g., whole transcriptome sequencing of mRNA encoding immune cell receptors. In some embodiments, the methods and systems described herein can also accomplish targeted genomic sequencing of nucleic acid molecules (e.g., DNA, RNA, and mRNA). In various embodiments, the methods and systems described herein accomplish genomic sequencing, for example, without limitation, single cell genomic sequencing of nucleic acid molecules (e.g., RNA and mRNA) encoding immune cell receptors of single cells, such as B cell receptors (BCRs) and T cell receptors (TCRs), and/or spatial genomic sequencing.

In various embodiments, the methods and systems described herein can include high-throughput sequencing technologies, e.g., high-throughput DNA and RNA sequencing technologies. In various embodiments, the methods and systems described herein can include high-throughput, higher accuracy short-read DNA and RNA sequencing technologies. In various embodiments, the methods and systems described herein can include long-read RNA sequencing, e.g., by sequencing cDNA transcripts in their entirety without assembly. In various embodiments, the methods and systems described herein can also, for example, segment long nucleic acid molecules into smaller fragments that can be sequenced using high-throughput, higher accuracy short-read sequencing technologies, and that segmentation is accomplished in a manner that allows the sequence information derived from the smaller fragments to retain the original long range molecular sequence context, i.e., allowing the attribution of shorter sequence reads to originating longer individual nucleic acid molecules. By attributing sequence reads to an originating longer nucleic acid molecule, one can gain significant characterization information for that longer nucleic acid sequence that one cannot generally obtain from short sequence reads alone. This long-range molecular context is not only preserved through a sequencing process but is also preserved through the targeted enrichment process used in targeted sequencing approaches.

In one or more embodiments, the methods and systems described herein are directed to single cell analysis (including single- and multi-modal analyses) of genomic sequencing of nucleic acids (e.g., RNA and mRNA) encoding immune cell receptors of single cells, such as B cell receptors (BCRs) and T cell receptors (TCRs). Single cell analysis, including single cell multi-modal analyses (e.g., single cell immune cell receptor sequencing combined with, for example, gene expression, protein expression, and/or antigen capture technologies), as well as processing and sequencing of nucleic acids, in accordance with the methods and systems described in the present application are described in further detail, for example, in U.S. Pat. Nos. 9,689,024; 9,701,998; 10,011,872; 10,221,442; 10,337,061; 10,550,429; 10,273,541; and U.S. Pat. Pub. 20180105808, which are all herein incorporated by reference in their entirety for all purposes and in particular for all written description, figures and working examples directed to processing nucleic acids and sequencing and other characterizations of genomic material.

The term “B cells”, also known as B lymphocytes, refer to a type of white blood cell of the small lymphocyte subtype. They function in the humoral immunity component of the adaptive immune system by expressing and/or secreting antibodies. Additionally, B cells present antigens (they are also classified as professional antigen-presenting cells (APCs)) and secrete cytokines. In mammals, B cells mature in the bone marrow, which is at the core of most bones. In birds, B cells mature in the bursa of Fabricius, an immune organ where they were first discovered by Chang and Glick, (B for bursa) and not from bone marrow as commonly believed. B cells, unlike the other two classes of lymphocytes, T cells and natural killer cells, express B cell receptors (BCRs) on their cell membrane or secrete their BCRs if they have differentiated into long-lived plasma cells. BCRs allow a B cell to bind to specific antigens, against which it will initiate an antibody response.

The term “T cell”, also known as T lymphocytes, refer to a type of an adaptive immune cell. T cells develops in the thymus gland, hence the name T cell, and play a central role in the immune response of the body. T cells can be distinguished from other lymphocytes by the presence of a T cell receptor (TCR) on the cell surface. These immune cells originate as precursor cells, derived from bone marrow, and then develop into several distinct types of T cells once they have migrated to the thymus gland. T cell differentiation continues even after they have left the thymus. T cells include, but are not limited to, helper T cells, cytotoxic T cells, memory T cells, regulatory T cells, and killer T cells. Helper T cells stimulate B cells to make antibodies and help killer cells develop. Based on the T cell receptor chain, T cells can also include T cells that express αβ TCR chains, T cells that express γδ TCR chains, as well as unique TCR co-expressors (i.e., hybrid αβ-γδ cells) that co-express the αβ and γδ TCR chains. T cells can also include engineered T cells that can attack specific cancer cells. A

patient's T cells can be collected and genetically engineered to produce chimeric antigen receptors (CAR). These engineered T cells are called CAR T cells, which forms the basis of the developing technology called CAR-T therapy. These engineered CAR T cells are grown by the billions in the laboratory and then infused into a patient's body, where the cells are designed to multiply and recognize the cancer cells that express the specific protein. This technology, also called adoptive cell transfer is emerging as a potential next-generation immunotherapy treatment.

T cells, such as the killer T cells can directly kill cells that have already been infected by a foreign invader. T cells can also use cytokines as messenger molecules to send chemical instructions to the rest of the immune system to ramp up its response. Activating T cells against cancer cells is the basis behind checkpoint inhibitors, a relatively new class of immunotherapy drugs that have recently been approved to treat lung cancer, melanoma, and other difficult cancers. Cancer cells often evade patrolling T cells by sending signals that make them seem harmless. Checkpoint inhibitors disrupt those signals and prompt the T cells to attack the cancer cells.

The term “naïve”, as used herein, can refer to B-lymphocytes or T-lymphocytes that have not yet reacted with an epitope of an antigen or that have a cellular phenotype consistent with that of a lymphocyte that has not yet responded to antigen-specific activation after clonal licensing.

The term “Fab”, also referred to as an antigen-binding fragment, refers to the variable portions of an antibody molecule with a paratope that enables the binding of a given epitope of a cognate antigen. The amino acid and nucleotide sequences of the Fab portion of antibody molecules are hypervariable. This is in contrast to the “Fc” or crystallizable fragment, which is relatively constant and encodes the isotype for a given antibody; this region can also confer additional functional capacity through processes such as antibody-dependent complement deposition, cellular cytotoxicity, cellular trogocytosis, and cellular phagocytosis.

The phrase “clonal selection” refers to the selection and activation of specific B lymphocytes and T lymphocytes by the binding of epitopes to B cell receptors or T cell receptors with a corresponding fit and the subsequent elimination (negative selection) or licensing for clonal expansion (positive selection) of a B or T lymphocyte after binding of an antigenic determinant. The phrase “clonal expansion” refers to the proliferation of B lymphocytes and T

lymphocytes activated by clonal selection in order to produce a clonal population of daughter cells with the same antigen specificity and functional capacity. In the case of T lymphocytes this antigen specificity is exact at the nucleotide and protein level and in the case of B lymphocytes this antigen specificity can be exact at the nucleotide and protein level or mutated relative to the patient population by mutations at the nucleotide level (and by extension the protein level). This enables the body to have sufficient numbers of antigen-specific lymphocytes to mount an effective immune response.

The phrase “T helper lymphocytes”, also referred to as helper cells, refer to a type of white blood cell that orchestrate the immune response and enhance the activities of the killer T-cells (those that destroy pathogens) and B cells (antibody and immunoglobulin producers).

The phrase “affinity maturation” refers to the gradual modification of the paratope and entire B cell receptor as a result of somatic hypermutation. B lymphocytes with higher affinity B cell receptors that can 1) bind the epitope more tightly and 2) therefore bind the epitope for a longer period of time are able to proliferate more and survive longer. These B cells can eventually differentiate into plasma cells, which secrete their antibodies and form the basis of serum-mediated immunity.

The phrase “heavy chain” refers to the large polypeptide subunit of an antibody (immunoglobulin). The first recombination event to occur is between one D and one J gene segment of the heavy chain locus. Any DNA between these two gene segments is deleted. This D-J recombination is followed by the joining of one V gene segment, from a region upstream of the newly formed DJ complex, forming a rearranged VDJ gene segment. All other gene segments between V and D segments are now deleted from the cell's genome. Primary tianscript (unspliced RNA) is generated containing the VDJ region of the heavy chain and both the constant mu and delta chains (Cμ and Cδ (i.e., the primary transcript contains the segments: V-D-J-Cμ-Cδ. The primary RNA is processed to add a polyadenylated (poly-A) tail after the Cp chain and to remove sequence between the VDJ segment and this constant gene segment. Translation of this mRNA leads to the production of the IgM heavy chain protein and the IgD heavy chain protein (its splice variant). Expression of the immunoglobulin heavy chain with one or more surrogate light chains constitutes the pre-B cell receptor that allows a B cell to undergo selection and maturation.

The phrase “light chain” refers to the small polypeptide subunit of an antibody (immunoglobulin). The kappa (κ) and lambda (λ) chains of the immunoglobulin light chain loci rearrange in a very similar way, except that the light chains lack a D segment. In other words, the first step of recombination for the light chains involves the joining of the V and J chains to give a VJ complex before the addition of the constant chain gene during primary transcription. Translation of the spliced mRNA for either the kappa or lambda chains results in formation of the Ig κ or Ig λ light chain protein. Assembly of the Ig μ heavy chain and one of the light chains results in the formation of membrane bound form of the immunoglobulin IgM that is expressed on the surface of the immature B cell. B cells may express up to two heavy chains and/or two light chains in respectively rare and uncommon instances through a phenomenon known as allelic inclusion. This phenomenon can only be directly observed using single-cell technologies, though it can be inferred with a degree of uncertainty using a combination of bulk sequencing technologies and probabilistic inference via an extension of the birthday paradox.

The phrase “complementarity-determining regions” (CDRs) refers to part of the variable chains in immunoglobulins (antibodies) and T cell receptors, generated by B cells and T cells respectively, where these molecules are particularly hypervariable. The antigen-binding site of most antibodies and T cell receptors is typically distributed across these CDRs, collectively forming a paratope. However, there are many documented examples of paratopes that enable antigen recognition that fall outside of the CDRs. As the most variable parts of the molecules, CDRs are crucial to the diversity of antigen specificities and immune cell receptor sequences generated by lymphocytes. V(D)J recombination is a genetic recombination mechanism that occurs in

developing lymphocytes during the early stages of T and B cell maturation. Through somatic recombination, this mechanism produces a highly diverse repertoire of antibodies/immunoglobulins and T cell receptors (TCRs) found in B cells and T cells, respectively. This process is a defining feature of the adaptive immune system and these receptors are defining features of adaptive immune cells.

V(D)J recombination occurs in the primary immune organs (bone marrow for B cells and thymus for T cells) and in a generally random fashion. The process leads to the rearranging of variable (V), joining (J), and in some cases, diversity (D) gene segments. As discussed above, the heavy chain possesses numerous V, D, and J gene segments, while the light chain possesses only V and J gene segments. The process ultimately results in novel amino acid sequences in the antigen-binding regions of immunoglobulins and TCRs that allow for the recognition of antigens from nearly all pathogens including, for example, bacteria, viruses, and parasites. Furthermore, the recognition can also be allergic in nature or may recognize host tissues and lead to autoimmunity.

Human antibody molecules or B cell receptors (BCRs) include both heavy and light chains, each of which contains both constant (C) and variable (V) regions, and are genetically encoded on three loci. The first is the immunoglobulin heavy locus on chromosome 14, containing the gene segments for the immunoglobulin heavy chain. The second is the immunoglobulin kappa (κ) locus on chromosome 2, containing the gene segments for part of the immunoglobulin light chain. The third is the immunoglobulin lambda (λ) locus on chromosome 22, containing the gene segments for the remainder of the immunoglobulin light chain.

Each heavy or light chain contains multiple copies of different types of gene segments for the variable regions of the antibody proteins. For example, the human immunoglobulin heavy chain region contains two C gene segments (Cμ and Cδ), 44 V gene segments, 27 D gene segments and 6 J gene segments. The number of given segments present in any individual can vary, as these gene segments are carried in haplotypes; for this reason, inference of both the alleles present within an individual and the germline sequence of those alleles is an important step in correctly identifying B cell clonotypes. The light chains possess two C gene segments (Cλ and Cκ) and numerous V and J gene segments, but do not have D gene segments. DNA rearrangement causes one copy of each type of gene segment to mate with any given lymphocyte, generating a substantial antibody repertoire. Approximately 10¹⁴ combinations are possible, with 1.5×10² to 3×10³ potentially removed via self-reactivity.

Accordingly, each naïve B cell makes an antibody with a unique Fab site through a series of gene recombinations, and later mutations, with the specific molecules of the given antibody attaching to the B cell's surface as a B cell receptor (BCR). These BCRs are then available to react with epitopes of an antigen.

When the immune system encounters an antigen, epitopes of that antigen will be presented to many B lymphocytes. B lymphocytes must first rearrange a heavy chain that enables pre-B cell receptor ligand binding. B lymphocytes that bind multivalent self-targets after rearrangement of the light chain too strongly are eliminated and die or undergo a secondary recombination event, while B cells that do not bind self-targets too strongly ar e licensed to exit the bone marrow. The latter becomes available to respond to non-self antigens and to undergo clonal expansion. This process is known as clonal selection.

Cytokines produced by activated CD4 T helper lymphocytes enable those activated B lymphocytes (B cells) to rapidly proliferate to produce large clones of thousands of identical B cells. More specifically, when under threat (i.e., via bacteria, virus, etc.), the body releases white blood cells by the immune system. CD4 T lymphocytes help the response to a threat by triggering the maturation of other types of white blood cell. They produce special proteins, called cytokines, have plural functions, including the ability to summon all of the other immune cells to the area, and also the ability to cause nearby cells to differentiate (become specialized) into mature B cells and T cells.

Accordingly, while only a few B cells in the body may have an antibody molecule that can bind a particular epitope, eventually many thousands of cells arc produced with the right specificity, allowing the body's immune system to act en masse. This is referred to as clonal expansion. Natural phenomena such as IgA deficiency and murine transgenic models have shown that there are multiple paths by which a B cell receptor can acquire novel antigen specificity even from a very limited repertoire through the processes of somatic hypermutation and affinity maturation.

As the B cells proliferate, they undergo affinity maturation as a result of somatic hypermutation. This allows the B cells to “fine-tune” the paratopes of the antibody to more effectively fit with the recognized epitopes. B cells with high affinity B cell receptors on their surface bind epitopes more tightly and for a longer period of time, which enables these cells to selectively proliferate. Over the course of this proliferation and expansion, these variant B cells differentiate into plasma cells that synthesize and secrete vast quantities of antibodies with Fab sites that fit the target epitopes very precisely.

The phrase “immune cell” refers to a cell that is part of the immune system and that helps the body fight infections and other diseases. Immune cells include innate immune cells (such as basophils, dendritic cells, neutrophils, etc.) that are the first line of body's defense and are deployed to help attack the invading foreign cells (e.g., cancer cells) and pathogens. The innate immune cells can quickly respond to foreign cells and pathogens to fight infection, battle a virus, or defend the body against bacteria. Immune cells can also include adaptive immune cells (such as lymphocytes including B cells and T cells). The adaptive immune cells can come into action when an invading foreign cells or pathogens slip through the first line of body's defense mechanism. The adaptive immune cells can take longer to develop, because their behaviors evolve from learned experiences, but they can tend to live longer than innate immune cells. Adaptive immune cells remember foreign invaders after their first encounter and fight them off the next time they enter the body. Both types of immune cells employ important natural defenses in helping the body fight foreign cells and pathogens for fighting infections and other diseases.

The immune cells of the disclosure can include, but are not limited to, neutrophils, eosinophils, basophils, mast cells, monocytes, macrophages, dendritic cells, natural killer cells, and lymphocytes (such as B cells and T cells). The immune cells of the disclosure can further include dual expresser cells or DE (such as unique dual-receptor-expressing lymphocytes that co-express functional B cell receptor (BCR) and T cell receptor (TCR)), cells with adaptive immune receptors that may diversify or may not diversify (including immune cells expressing a chimeric antigen receptor with a fixed nucleotide sequence or with the capacity to mutate), and TCR co-expressors (i.e., hybrid αβ-γδ cells) that co-express both αβ and γϵ TCR chains.

The phrase “Immune cell receptor”, “immune receptor”, or “immunologic receptor” refers to a receptor or immune cell receptor sequence, usually on a cell membrane, which can recognize components of pathogenic microorganisms (e.g., components of bacterial cell wall, bacterial flagella or viral nucleic acids) and foreign cells (e.g., cancer cells), which are foreign and not found naturally on the host cells, or binds to a target molecule (for example, a cytokine), and causes a response in the immune system. The immune cell receptors of the immune system can include, but are not limited to, pattern recognition receptors (PRRs), Toll-like receptors (TLRs), killer activated and killer inhibitor receptors (KARs and KIRs), complement receptors, Fc receptors, B cell receptors, and T cell receptors.

The phrase “immune cell receptor sequences” of an immune cell receptor include both heavy and light chains, each of which contains both constant (C) and variable (V) regions. For example, B cell receptors (BCRs) or B cell receptor sequences (including human antibody molecules) comprise of immunoglobulin heavy and light chains, each of which contains both constant (C) and variable (V) regions. Each heavy or light chain not only contains multiple copies of different types of gene segments for the variable regions of the antibody proteins, but also contains constant regions. For example, the BCR or human immunoglobulin heavy chain contains two (2) constant (Constant mu (Cμ) and delta (Cδ)) gene segments and 44 Variable (V) gene segments, plus twenty seven (27) Diversity (D) gene segments, and six (6) Joining (J) gene segments. The BCR light chains also possess two (2) constant gene segments ((Constant lambda (Cλ) and kappa (Cκ) and numerous V and J gene segments, but do not have any D gene segments. DNA rearrangement (i.e., recombination events) in developing B cells can cause one copy of each type of gene segment to go in any given lymphocyte, generating an enormous antibody repertoire.

Accordingly, the primary transcript (unspliced RNA) of a BCR heavy chain can be generated containing the VDJ region of the heavy chain and both the constant mu and delta chains (Cμ and Cδ), i.e., the heavy chain primary transcript can contain the segments: V-D-J-Cμ-Cδ). In case of the B cell receptor and human immunoglobulin light chain, the first step of recombination for the light chains involves the joining of the V and J chains to give a VJ complex before the addition of the constant chain gene during primary transcription. Translation of the spliced mRNA for either the constant κ (Cκ) or λ (Cλ) chains results in formation of the Ig κ or Igλ light chain protein.

Most T cell receptors (TCR) are composed of an alpha (α) chain and a beta (β) chain, each of which contains both constant (C) and variable (V) regions. Thus, the most common type of a T cell receptor is called an alpha-beta TCR because it is composed of two different chains, one a-chain and one beta β-chain. A less common type of TCR is the gamma-delta TCR. which contains a different set of chains, one gamma (γ) chain and one delta (δ) chain. The T cell receptor genes are similar to immunoglobulin genes for the BCR and undergo similar DNA rearrangement (i.e., recombination events) in developing T cells as for the B cells. For example, the alpha-beta TCR genes also contain multiple V, D, and J gene segments in their beta chains and V and J gene segments in their alpha chains, which are re-arranged during the development of the T cells to provide a cell with a unique T cell antigen receptor. Similar to the alpha-beta TCRs, the TCR-γ chain is produced by V-J recombinations and can contain Vγ-Jγ gene segments and constant domain (Cγ) genes resulting in a Vγ-Jγ-Cγ sequence of the TCR y-chain, while the TCR-δ chain is produced using V-D-J recombinations, and can contain Vδ-Dδ-Jδ gene segments and constant domain (Cδ) genes resulting in a Vδ-Dδ-Jδ-Cδ sequence of the TCR 5-chain.

The phrase “immune cell receptor constant region sequence” or “immune receptor constant region sequence” refers to the constant region or constant region sequence of an immune cell receptor. For example, the immune cell receptor constant region sequence or immune receptor constant region sequence can include, but is not limited to, the constant mu (Cμ) and delta (Cδ) region genes and sequences of a BCR and immunoglobulin heavy chain, the constant lambda (Cλ) and kappa (Cκ) region genes and sequences of a BCR and immunoglobulin light chain, the alpha constant (Cα) region genes and sequences of a TCR a-chain sequence, the beta constant (Cβ) region genes and sequences of a TCR β-chain sequence, the gamma constant (Cγ) region genes and sequences of a TCR γ-chain sequence, and the delta constant (Cδ) region genes and sequences of a TCR δ-chain sequence.

### II. Introduction

With this understanding of the immune cell's purpose in fighting off foreign antigens, the pharmaceutical industry has strongly focused on designing pharmaceutical compositions with the ability to expand antibody lineages directed towards specific B cells or T cells with shared antigen specificity. To most effectively determine the efficacy of a vaccine or antitumor antibody therapy, it is useful to be able to accurately identify cell members of a clonotype, which potentially share common or similar antigen specificity. The pharmaceutical industry has also directed its efforts to isolate antibodies and antibody lineages against non-foreign targets for the purpose of developing antibody-based therapeutics for a broad array of disease states including autoimmune disease (anti-inflammatory targets), cancer (checkpoint inhibitors and other targets), and other conditions such as osteoporosis. Similarly, knowing the fine specificities of different antibody lineages elicited by a vaccine is useful to understand serum neutralization profiles and global epitope maps of an antigen. This same concept applies to understanding how a patient's adaptive immune system can render antibody drugs such as adalimumab ineffective through the emergence of anti-drug antibodies and distinct anti-drug antibody lineage.

Available approaches to group and visualize immune cell receptor sequences do not enable interactive analysis for displaying a large amount of information about the single cells within a clonotype in a compact and readily interpretable display. Therefore, in accordance with various embodiments, various systems and methods are provided that display large amounts of information related to clonotype groupings for B cells or T cells in a dynamic, interactive and compact graphic representation such as graphical user interface (GUI). In accordance with various embodiments, visualization methods can be used for displaying the location of specific mutations, the type of amino acids present, the abundance (e.g., quantities or frequency) and location of protein motifs that may pose developability challenges for therapeutic purposes so one can easily relate all of this information in a compact space to the phylogenetic/developmental relationships between the cells in each clonotype.

The visualization methods and systems described herein may be used to identify and develop therapeutics, e.g., for severe acute respiratory syndrome coronavirus 2 (SARS-CoV-2), a newly-identified emerging coronavirus causing an acute respiratory distress syndrome known as COVID-19 that is similar to severe acute respiratory syndrome (SARS) caused by the closely related SARS-CoV. The visualization methods and systems described herein may be used to identify or otherwise provide anti-SARS-CoV-2 S antibodies and antigen-binding fragments thereof, and to provide therapeutic methods of using such antibodies and fragments for treating viral infections based on SARS-CoV-2. For example, one or more of the embodiments described herein provide visualization schemas that enable faster and more efficient isolation of antibodies or antigen-binding fragments thereof, that bind specifically to a spike (S) protein of severe acute respiratory syndrome coronavirus 2 (SARS-CoV-2).

### III. Barcoding and Sequencing Methodologies

Systems and methods to visualize and present these data for interactive analysis and interpretation are useful and readily applied to immune cell data generated using barcoding and sequencing technologies. Examples include single cell sequencing technologies such as non-droplet and droplet-based microfluidic single cell sequencing (e.g., single cell genomic sequencing) technologies, array-based microwell- and nanowell-based single cell sequencing technologies (e.g., array-based microwell- and nanowell based single cell genomic sequencing), and in situ sequencing technologies. Other examples include spatial analysis methodologies, e.g., spatially indexed single cell technologies, which arc described further herein.

Any known sequencing methods (e.g., including single cell sequencing methods and spatial analysis methodologies) can be used to provide immune cell data (e.g., single immune cell sequencing data or spatial data) in various embodiments. In various embodiments, with single cell sequencing methods, single cells can be separated into partitions such as droplets or wells, wherein each partition comprises a single cell with a known identifier like a barcode. The barcode can be attached to a support, for example, a bead, such as a solid bead or a gel bead.

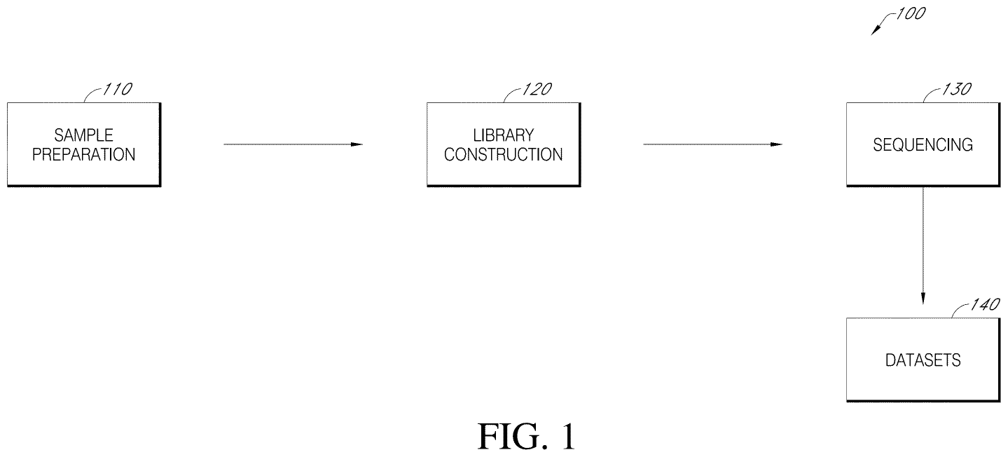

In accordance with various embodiments, a general schematic workflow is provided in FIG. 1 to illustrate a non-limiting example process for using immune cell sequencing technology (e.g., single cell or spatial analysis methodologies) to generate immune cell data such as, for example, without limitation, immune cell sequencing data, antigen binding data, or a combination thereof. Such immune sequencing data and antigen binding data can be used for identifying V(D)J information, clonotype information, and antigen specificity in accordance with various embodiments. The workflow can include various combinations of features, whether it be more or less features than that illustrated in FIG. 1. As such, FIG. 1 simply illustrates one example of a possible workflow. The workflow provided in FIG. 1 may include using, for example, single cell sequencing methodologies or spatial analysis methodologies. Accordingly, the single cell methodologies described below with respect to FIG. 1 are merely examples of how the workflow in FIG. 1 may be implemented and are not meant to limiting. Spatial analysis methodologies that may be included in the workflow in FIG. 1 are described further below.

### Sample Preparation and Processing

The workflow provided in FIG. 1 depicts sample preparation/processing at 110, which includes, without limitation, partition-based approaches for processing single cells or their components, or spatial array based methodologies, described further herein.

In exemplary partition-based approaches, single cells may be partitioned into wells (e.g., a microwell array) or into a plurality of droplets-in-an-emulsion (e.g., 10× Genomics). Single cells of the immune system can be partitioned and processed to yield barcoded nucleic acid molecules as described in US 2018-0105808 A1, which is incorporated herein by reference in its entirety.

In general, a partition (from a plurality of partitions comprising a plurality of single cells) can comprise a single cell (or the lysate of a single cell) and a plurality of nucleic acid barcode molecules. In one embodiment, the partition further comprises a support (e.g., a bead such as a gel bead) which comprises the plurality of nucleic acid barcode molecules. In another embodiment, the plurality of nucleic acid barcode molecules comprise a common barcode sequence.

The partition can further comprise additional reagents to allow for processing of the single cell and the generation of barcoded nucleic acid molecules from the single cell or components thereof, e.g., nucleic acid components including RNA, such as mRNA, and DNA. Such barcoded nucleic acid molecules comprise the common barcode sequence or a complement thereof. Other aspects of partition-based barcoding of nucleic acid molecules are described in U.S. Pat. Nos. 10,323,278, 10,550,429, 10,815,525, 10,725,027, 10,343,166, 10,5834,40, as well as U.S. Pat. Pubs. US2018/01058080A1, US2018/0179590A1, and US2019/0367969A1, and Published International PCT Application No. WO 2019/040637, each of which is incorporated herein by reference in its entirety.

In various embodiments, this sample preparation step 110 can result in partitioning of single cells comprising analytes of interest (e.g., mRNA) into a plurality of individual partitions (e.g., droplets or wells), wherein the plurality of partitions comprises a subset of partitions, each comprising a single cell and a plurality of nucleic acid barcode molecules comprising a common barcode sequence. The plurality of nucleic acid barcode molecules can be provided as part of a support (e.g., a single bead such as a single gel bead). Within a partition of the subset of partitions, a plurality of barcoded nucleic acid molecules is generated, wherein a barcoded nucleic acid molecule of the plurality of barcoded nucleic acid molecules comprises the common barcode sequence or complement thereof and a sequence corresponding to a nucleic acid analyte (e.g., an mRNA molecule) from the single cell. Further details can be found in U.S. Pat. Pub. 2018/0105808, U.S. Pat. Nos. 10,323,278, 10,550,429 and 10,815,525, U.S. Published Application Nos. US2018/0179590A1 and, US2019/0367969A1, and Published International PCT Application No. WO 2019/040637, each of which is incorporated herein by reference in its entirety.

In one embodiment, the sample preparation step 110 further includes lysing the single cells and barcoding the cellular analytes (e.g., mRNA molecules) to produce a plurality of barcoded nucleic acid molecules that can be sequenced to yield information about the cells (e.g., determining gene expression).

Upon generation of the plurality of partitions as described above, nucleic acid barcode molecules in a partition are used to generate a plurality of barcoded nucleic acid molecules. In one embodiment, the plurality of nucleic acid barcode molecules is provided as part of a support and released from the support to generate the plurality of barcoded nucleic acid molecules using the released nucleic acid barcode molecules and nucleic acid molecules from the cell.

In one embodiment, the support is a bead. In another embodiment, the bead is a gel bead that can be degraded within the partition to release the nucleic acid barcode molecules. The released nucleic acid barcode molecules, as well as nucleic acid molecules from the cell (e.g., mRNA) and reagents (e.g., reverse transcription (RT) reagents) arc used to perform a nucleic acid extension reaction (e.g., reverse transcription of polyadenylated mRNA) to generate the barcoded nucleic acid molecules within the partition. As a result, the barcoded nucleic acid molecules generated in the partition can comprise the common barcode sequence (as described herein), thereby allowing the sequencing reads to be mapped back to their original single cells of origin.

In various embodiments, the barcoded nucleic acid molecules may be removed from the partitions for further processing. In one embodiment, where the partitions arc droplets in an emulsion (e.g., GEMs), a plurality of droplets are broken, and their contents pooled. In another embodiment, the pooled contents comprise barcoded cDNA molecules that comprise barcode sequences from their respective partitions. In one other embodiment, barcoded cDNA molecules are processed in bulk to complete library preparation for sequencing (e.g., next generation high throughput sequencing), as described in detail below. In various embodiments, following the amplification process, leftover biochemical reagents can be removed from the post-partition reaction mixture.

Various protocols known in the art can be employed to generate suspensions for use with some embodiments herein. Suspensions can be generated from any cells. Such cells may include, but are not limited to, cells from fresh and cryopreserved cell lines, e.g., human and mouse cell lines, as well as more fragile primary cells. In various embodiments, such cells may include, any eukaryotic cells, i.e., a eukaryotic cell with a chromatin structure. In various embodiments, such cells may include, but are not limited to, immune cells (e.g., B cells and T cells), peripheral blood mononuclear cells (PBMCs), bone marrow mononuclear Cells (BMNICs), or any lymphocytes.

### Library Construction

The workflow 100 provided in FIG. 1 further includes library construction at step 120 based on single cell or spatial analysis. In one or more embodiments, in the library construction step of workflow 100, a library containing a plurality of double-stranded DNA fragments is generated. These double-stranded DNA fragments can be utilized for completing the subsequent sequencing step. Detail related to the library construction, in accordance with various embodiments disclosed herein, is provided below. Further details can be found in U.S. Pat. Pub. 2018/0105808, which is incorporated herein by reference in its entirety.

In accordance with various embodiments disclosed herein, an adapter sequence) and optionally a sample index (SI) sequence can be added during the library construction step via PCR to generate the library, which contains a plurality of double stranded DNA fragments. In accordance with various embodiments herein, the sample index sequences can each comprise one or more oligonucleotides. In one embodiment, the sample index sequences can each comprise four oligonucleotides. In various embodiments, when analyzing the immune cell data (e.g., single cell sequencing data or spatial data) for a given sample (e.g., single cells or tissue sample, respectively), the reads associated with all four of the oligonucleotides in the sample index can be combined for identification of a sample.

For example, barcoded cDNA molecules recovered from the plurality of partitions can be used as templates for multiplexed PCR to produce a single cell library. Various embodiments of single cell sequencing technology within the disclosure can at least include platforms such as One Sample, One GEM Well, One Flowcell; One Sample, One GEM well, Multiple Flowcells; One Sample, Multiple GEM Wells, One Flowcell; Multiple Samples, Multiple GEM Wells, One Flowcell; and Multiple Samples, Multiple GEM Wells, Multiple Flowcells platform. Accordingly, various embodiments within the disclosure can include sequence dataset from one or more samples, samples from one or more donors, and multiple libraries from one or more donors.

### Sequencing

The workflow 100 provided in FIG. 1 further includes a sequencing step 130 to generate immune cell data that includes a data set 140 that provides immune cell receptor information, e.g., on a single cell basis or a spatial basis. In this step, the library can be sequenced to generate a plurality of sequencing data. The fully constructed library can be sequenced according to a suitable sequencing technology, such as a next-generation sequencing protocol, to generate the sequencing data. In non-limiting exemplary embodiments, the next-generation sequencing protocol utilizes the llumina® sequencer, Pacific Biosciences (PacBio®), Oxford Nanopore Technologies (ONT) sequencing platforms for generating the sequencing data. It is understood that other next-generation sequencing protocols, platforms, and sequencers such as, e.g., MiSeq™, NextSeq™ 500/550 (High Output), Hi Seq 2500™ (Rapid Run), Hi Seq™ 3000/4000, and NovaSeg™, can be also used with various embodiments herein. Further details can be found in U.S. Pat. Pub. 2018/0105808, which is incorporated herein by reference in its entirety.

The various embodiments, systems and methods within the disclosure further include processing and inputting the sequence data. A compatible format of the sequencing data of the various embodiments herein can be a FASTQ file. Other file formats for inputting the sequence data are also contemplated within the disclosure herein. Various software tools within the embodiments herein can be employed for processing and inputting the sequencing output data into input files for the downstream data analysis workflow. It is understood that various systems and methods with the embodiments herein are contemplated and can be employed to simultaneously analyze the inputted single cell sequencing data or spatial data for sequence analysis in accordance with various embodiments.

### Spatial Analysis Methodologies

Spatial analysis methodologies and compositions described herein can provide a vast amount of analyte and/or expression data for a variety of analytes within a biological sample at high spatial resolution, while retaining native spatial context. Spatial analysis methods and compositions can include, e.g., the use of a capture probe including a spatial barcode (e.g., a nucleic acid sequence that provides information as to the location or position of an analyte within a cell or a tissue sample (e.g., mammalian cell or a mammalian tissue sample) and a capture domain that is capable of binding to an analyte (e.g., a protein and/or a nucleic acid) produced by and/or present in a cell. Spatial analysis methods and compositions can also include the use of a capture probe having a capture domain that captures an intermediate agent for indirect detection of an analyte. For example, the intermediate agent can include a nucleic acid sequence (e.g., a barcode) associated with the intermediate agent. Detection of the intermediate agent is therefore indicative of the analyte in the cell or tissue sample.

Non-limiting aspects of spatial analysis methodologies and compositions are described in U.S. Pat. Nos. 10,774,374, 10,724,078, 10,480,022, 10,059,990, 10,041,949, 9,879,313, 9,783,841, 9,727,810. 9,593,365, 8,951,726, 8,604,182, 7,709,198, U.S. Patent Application Publication Nos. 2020/239946, 2020/080136, 2020/0277663, 2020/024641, 2019/330617, 2019/264268, 2020/256867, 2020/224244, 2019/194709, 2019/161796, 2019/085383, 2019/055594, 2018/216161, 2018/051322, 2018/0245142, 2017/241911, 2017/089811, 2017/067096, 2017/029875, 2017/0016053, 2016/108458, 2015/000854, 2013/171621, WO 2018/091676, WO 2020/176788, Rodrigues et al., Science 363(6434): 1463-1467, 2019; Lee etal., Nat. Protoc. 10(3):442-458, 2015; Trejo etal., PLOS ONE 14(2):e0212031, 2019; Chen et al., Science 348(6233) :aaa6090, 2015; Gao et al., BMC Biol. 15:50, 2017; and Gupta etal., Nature Biotechnol. 36:1197-1202, 2018; the Visium Spatial Gene Expression Reagent Kits User Guide (e.g., Rev D, dated October 2020), and/or the Visium Spatial Tissue Optimization Reagent Kits User Guide (e.g., Rev D, dated October 2020), both of which are available at the 10× Genomics Support Documentation website, and can be used herein in any combination. Further non-limiting aspects of spatial analysis methodologies and compositions are described herein.

Array-based spatial analysis methods involve the transfer of one or more analytes from a biological sample to an array of features on a substrate, where each feature is associated with a unique spatial location on the array. Subsequent analysis of the transferred analytes includes determining the identity of the analytes and the spatial location of the analytes within the biological sample. The spatial location of an analyte within the biological sample is determined based on the feature to which the analyte is bound (e.g., directly or indirectly) on the array, and the feature's relative spatial location within the array.

A “capture probe” refers to any molecule capable of capturing (directly or indirectly) and/or labelling an analyte (e.g., an analyte of interest) in a biological sample. In some embodiments, the capture probe is a nucleic acid or a polypeptide. In some embodiments, the capture probe includes a barcode (e.g., a spatial barcode and/or a unique molecular identifier (UMI)) and a capture domain). In some embodiments, a capture probe can include a cleavage domain and/or a functional domain (e.g., a primer-binding site, such as for next-generation sequencing (NGS)).

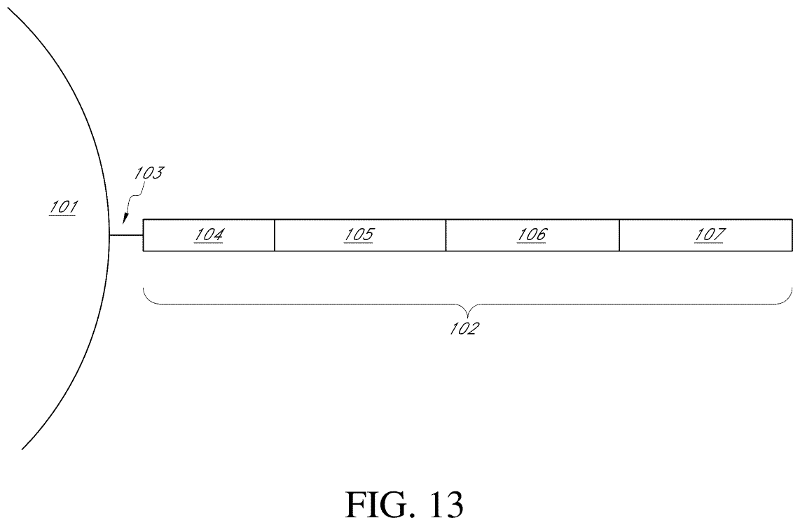

FIG. 13 is a schematic diagram showing an exemplary capture probe, as described herein. As shown in FIG. 13, the capture probe 102 is optionally coupled to a feature 101 by a cleavage domain 103, such as a disulfide linker. The capture probe can include a functional sequence 104 that is useful for subsequent processing. The functional sequence 104 can include all or a part of sequencer specific flow cell attachment sequence (e.g., a P5 or P7 sequence), all or a part of a sequencing primer sequence, (e.g., a R1 primer binding site, a R2 primer binding site), or combinations thereof. The capture probe can also include a spatial barcode 105. The capture probe can also include a unique molecular identifier (UMI) sequence 106. While FIG. 13 shows the spatial barcode 105 as being located upstream (5′) of UMI sequence 106, it is to be understood that capture probes wherein UMI sequence 106 is located upstream (5′) of the spatial barcode 105 is also suitable for use in any of the methods described herein. The capture probe can also include a capture domain 107 to facilitate capture of a target analyte. The capture domain can have a sequence complementary to a sequence of a nucleic acid analyte. The capture domain can have a sequence complementary to a connected probe described herein. The capture domain can have a sequence complementary to a capture handle sequence present in an analyte capture agent. The capture domain can have a sequence complementary to a splint oligonucleotide. Such splint oligonucleotide, in addition to having a sequence complementary to a capture domain of a capture probe, can have a sequence of a nucleic acid analyte, a sequence complementary to a portion of a connected probe described herein, and/or a capture handle sequence described herein.

The functional sequences can generally be selected for compatibility with any of a variety of different sequencing systems, e.g., Ion Torrent Proton or PGM, Illumina sequencing instruments, PacBio, Oxford Nanopore, etc., and the requirements thereof. In some embodiments, functional sequences can be selected for compatibility with non-commercialized sequencing systems. Examples of such sequencing systems and techniques, for which suitable functional sequences can be used, include (but are not limited to) Ion Torrent Proton or PGM sequencing, Illumina sequencing, PacBio SMRT sequencing, and Oxford Nanopore sequencing. Further, in some embodiments, functional sequences can be selected for compatibility with other sequencing systems, including non-commercialized sequencing systems.

Referring again to FIG. 13, in some embodiments, the spatial barcode 105 and functional sequences 104 are common to all of the probes attached to a given feature. In some embodiments, the UMI sequence 106 of a capture probe attached to a given feature is different from the UMI sequence of a different capture probe attached to the given feature.

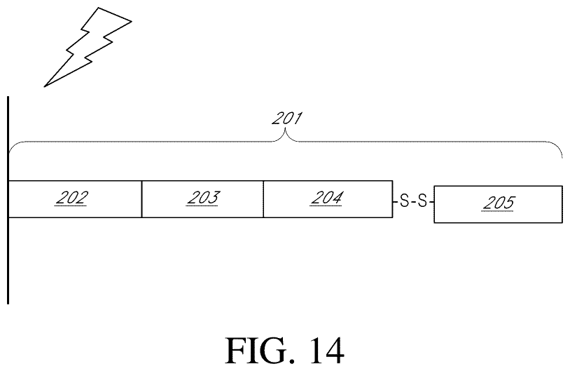

FIG. 14 is a schematic illustrating a cleavable capture probe, wherein the cleaved capture probe can enter into a non-permeabilized cell and bind to analytes within the sample. As shown in FIG. 14, the capture probe 201 contains a cleavage domain 202, a cell penetrating peptide 203, a reporter molecule 204, and a disulfide bond (—S—S—). 205 represents all other parts of a capture probe, for example a spatial barcode and a capture domain.

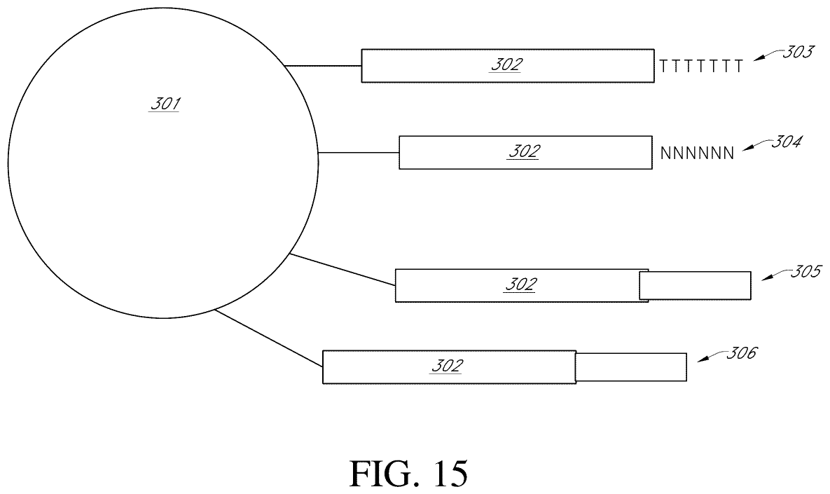

FIG. 15 is a schematic diagram of an exemplary multiplexed spatially-barcoded feature. In FIG. 15, the feature 301 can be coupled to spatially-barcoded capture probes, wherein the spatially-barcoded probes of a particular feature can possess the same spatial barcode, but have different capture domains designed to associate the spatial barcode of the feature with more than one target analyte. For example, a feature may be coupled to four different types of spatially-barcoded capture probes, each type of spatially-barcoded capture probe possessing the spatial barcode 302. One type of capture probe associated with the feature includes the spatial barcode 302 in combination with a poly(T) capture domain 303, designed to capture mRNA target analytes. A second type of capture probe associated with the feature includes the spatial barcode 302 in combination with a random N-mer capture domain 304 for gDNA analysis. A third type of capture probe associated with the feature includes the spatial barcode 302 in combination with a capture domain complementary to a capture handle sequence of an analyte capture agent of interest 305. A fourth type of capture probe associated with the feature includes the spatial barcode 302 in combination with a capture domain that can specifically bind a nucleic acid molecule 306 that can function in a CRISPR assay (e.g., CRISPR/Cas9). While only four different capture probe-barcoded constructs are shown in FIG. 15, capture-probe barcoded constructs can be tailored for analyses of any given analyte associated with a nucleic acid and capable of binding with such a construct. For example, the schemes shown in FIG. 15 can also be used for concurrent analysis of other analytes disclosed herein, including, but not limited to: (a) mRNA, a lineage tracing construct, cell surface or intracellular proteins and metabolites, and gDNA; (b) mRNA, accessible chromatin (e.g., ATAC-seq, DNase-seq, and/or MNase-seq) cell surface or intracellular proteins and metabolites, and a perturbation agent (e.g., a CRISPR crRNA/sgRNA, TALEN, zinc finger nuclease, and/or antisense oligonucleotide as described herein); (c) mRNA, cell surface or intracellular proteins and/or metabolites, a barcoded labelling agent (e.g., the MHC multimers described herein), and a V(D)J sequence of an immune cell receptor (e.g., T-cell receptor) or antigen binding molecule (ABM).

There are at least two methods to associate a spatial barcode with one or more neighboring cells, such that the spatial barcode identifies the one or more cells, and/or contents of the one or more cells, as associated with a particular spatial location. One method is to promote analytes or analyte proxies (e.g., intermediate agents) out of a cell and towards a spatially-barcoded array (e.g., including spatially-barcoded capture probes). Another method is to cleave spatially-barcoded capture probes from an array and promote the spatially-barcoded capture probes towards and/or into or onto the biological sample.

In some cases, capture probes may be configured to prime, replicate, and consequently yield optionally barcoded extension products from a template (e.g., a DNA or RNA template, such as an analyte or an intermediate agent (e.g., a connected probe (e.g., a ligation product) or an analyte capture agent), or a portion thereof), or derivatives thereof (see, e.g., Section (II)(b)(vii) of WO 2020/176788 and/or U.S. Patent Application Publication No. 2020/0277663 regarding extended capture probes). In some cases, capture probes may be configured to form a connected probe (e.g., a ligation product) with a template (e.g., a DNA or RNA template, such as an analyte or an intermediate agent, or portion thereof), thereby creating ligations products that serve as proxies for a template.

As used herein, an “extended capture probe” refers to a capture probe having additional nucleotides added to the terminus (e.g., 3′ or 5′ end) of the capture probe thereby extending the overall length of the capture probe. For example, an “extended 3′ end” indicates additional nucleotides were added to the most 3′ nucleotide of the capture probe to extend the length of the capture probe, for example, by polymerization reactions used to extend nucleic acid molecules including templated polymerization catalyzed by a polymerase (e.g., a DNA polymerase or a reverse transcriptase). In some embodiments, extending the capture probe includes adding to a 3′ end of a capture probe a nucleic acid sequence that is complementary to a nucleic acid sequence of an analyte or intermediate agent specifically bound to the capture domain of the capture probe. In some embodiments, the capture probe is extended using reverse transcription. In some embodiments, the capture probe is extended using one or more DNA polymerases. The extended capture probes include the sequence of the capture probe and the sequence of the spatial barcode of the capture probe.

In some embodiments, extended capture probes are amplified (e.g., in bulk solution or on the array) to yield quantities that are sufficient for downstream analysis, e.g., via DNA sequencing. In some embodiments, extended capture probes (e.g., DNA molecules) act as templates for an amplification reaction (e.g., a polymerase chain reaction).

Additional variants of spatial analysis methods, including in some embodiments, an imaging step, are described in Section (II)(a) of WO 2020/176788 and/or U.S. Patent Application Publication No. 2020/0277663. Analysis of captured analytes (and/or intermediate agents or portions thereof), for example, including sample removal, extension of capture probes, sequencing (e.g., of a cleaved extended capture probe and/or a cDNA molecule complementary to an extended capture probe), sequencing on the array (e.g., using, for example, in situ hybridization or in situ ligation approaches), temporal analysis, and/or proximity capture, is described in Section (II)(g) of WO 2020/176788 and/or U.S. Patent Application Publication No. 2020/0277663. Some quality control measures arc described in Section (II)(h) of WO 2020/176788 and/or U.S. Patent Application Publication No. 2020/0277663.

For spatial array-based methods, a substrate may function as a support for direct or indirect attachment of capture probes to features of the array. A “feature” is an entity that acts as a support or repository for various molecular entities used in spatial analysis. In some embodiments, some or all of the features in an array are functionalized for analyte capture. Exemplary substrates are described in Section (II)(c) of WO 2020/176788 and/or U.S. Patent Application Publication No. 2020/0277663. Exemplary features and geometric attributes of an array can be found in Sections (II)(d)(i), (II)(d)(iii), and (II)(d)(iv) of WO 2020/176788 and/or U.S. Patent Application Publication No. 2020/0277663.

Generally, analytes and/or intermediate agents (or portions thereof) can be captured when contacting a biological sample (e.g., a tissue sample) with a substrate including capture probes (e.g., a substrate with capture probes embedded, spotted, printed, fabricated on the substrate, or a substrate with features (e.g., beads, wells) comprising capture probes). As used herein, “contact,” “contacted,” and/or “contacting,” a biological sample with a substrate refers to any contact (e.g., direct or indirect) such that capture probes can interact (e.g., bind covalently or non-covalently (e.g., hybridize)) with analytes from the biological sample. Capture can be achieved actively (e.g., using electrophoresis) or passively (e.g., using diffusion). Analyte capture is further described in Section (II)(e) of WO 2020/176788 and/or U.S. Patent Application Publication No. 2020/0277663.

In some cases, spatial analysis can be performed by attaching and/or introducing a molecule (e.g., a peptide, a lipid, or a nucleic acid molecule) having a barcode (e.g., a spatial barcode) to a biological sample (e.g., a tissue sample). In some embodiments, a plurality of molecules (e.g., a plurality of nucleic acid molecules) having a plurality of barcodes (e.g., a plurality of spatial barcodes) are introduced to a biological sample (e.g., to a plurality of cells in a biological sample) for use in spatial analysis. In some embodiments, after attaching and/or introducing a molecule having a barcode to a biological sample, the biological sample can be physically separated (e.g., dissociated) into single cells or cell groups for analysis. Some such methods of spatial analysis are described in Section (III) of WO 2020/176788 and/or U.S. Patent Application Publication No. 2020/0277663.

During analysis of spatial information, sequence information for a spatial barcode associated with an analyte is obtained, and the sequence information can be used to provide information about the spatial distribution of the analyte in the biological sample. Various methods can be used to obtain the spatial information. In some embodiments, specific capture probes and the analytes they capture are associated with specific locations in an array of features on a substrate. For example, specific spatial barcodes can be associated with specific array locations prior to array fabrication, and the sequences of the spatial barcodes can be stored (e.g., in a database) along with specific array location information, so that each spatial barcode uniquely maps to a particular array location.

Alternatively, specific spatial barcodes can be deposited at predetermined locations in an array of features during fabrication such that at each location, only one type of spatial barcode is present so that spatial barcodes are uniquely associated with a single feature of the array. Where necessary, the arrays can be decoded using any of the methods described herein so that spatial barcodes are uniquely associated with array feature locations, and this mapping can be stored as described above.

When sequence information is obtained for capture probes and/or analytes during analysis of spatial information, the locations of the capture probes and/or analytes can be determined by referring to the stored information that uniquely associates each spatial barcode with an array feature location. In this manner, specific capture probes and captured analytes are associated with specific locations in the array of features. Each array feature location represents a position relative to a coordinate reference point (e.g., an array location, a fiducial marker) for the array. Accordingly, each feature location has an “address” or location in the coordinate space of the array.

Exemplary spatial methodologies for generating immune cell data (e.g., spatial datasets of at least one of immune cell receptors, antibodies, or fragments thereof from a tissue sample) are further described in WO2021247568 and WO2021247543, which are hereby incorporated by reference in their entirety. Such immune cell data may be obtained from tissue samples, e.g., tissue sections. The tissue section can be a fresh frozen tissue section, a fixed tissue section, or an FFPE tissue section. In some embodiments, the tissue sample is fixed and/or stained (e.g., a fixed and/or stained tissue section). Non-limiting examples of stains include histological stains (e.g., hematoxylin and/or eosin) and immunological stains (e.g., fluorescent stains). In some embodiments, a biological sample (e.g., a fixed and/or stained biological sample) can be imaged. Tissue samples are also described in Section (I)(d) of WO 2020/176788 and/or U.S. Patent Application Publication No. 2020/0277663.

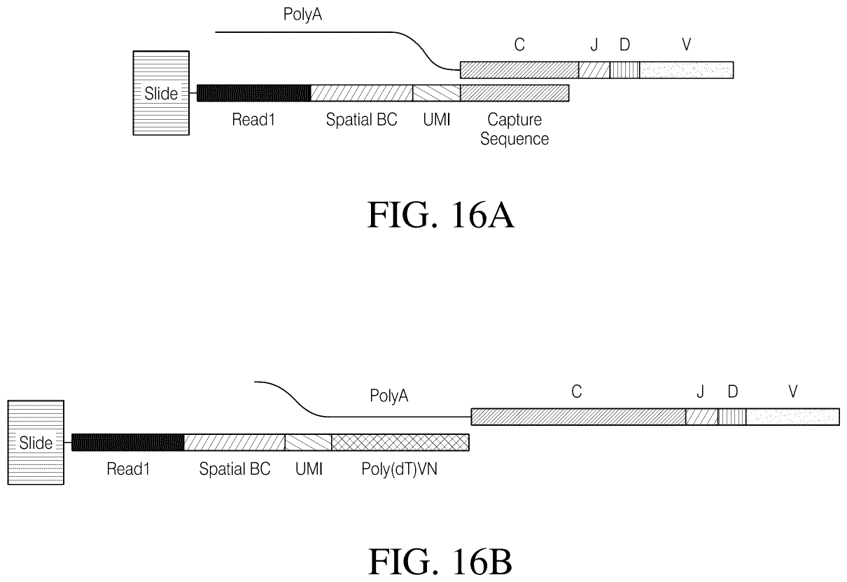

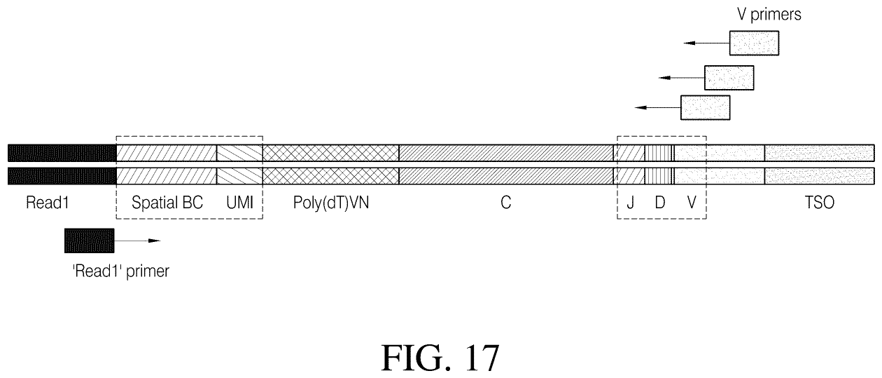

An exemplary embodiment of a spatial methodology for generating immune cell data (e.g., sequence data for an antigen binding molecule (ABM)) is depicted in FIG. 16A. An exemplary capture probe with a capture sequence that specifically binds to a nucleic acid sequence encoding a constant region of an ABM is depicted in FIG. 16A. In some embodiments, the ABM is selected from: a TCR alpha chain, a TCR beta chain, a TCR gamma chain, a TCR delta chain an immunoglobulin kappa light chain, an immunoglobulin lambda light chain, an immunoglobulin heavy chain. In some embodiments, the first capture sequence binds specifically to a nucleic acid sequence encoding a constant region of the T cell receptor alpha chain. In some embodiments, the first capture sequence binds specifically to a nucleic acid sequence encoding a constant region of the T cell receptor beta chain. In some embodiments, the first capture sequence binds specifically to a nucleic acid sequence encoding a constant region of the T cell receptor delta chain. In some embodiments, the first capture sequence binds specifically to a nucleic acid sequence encoding a constant region of the T cell receptor gamma chain. In some embodiments, the first capture sequence binds specifically to a nucleic acid sequence encoding a constant region of the immunoglobulin kappa light chain. In some embodiments, the first capture sequence binds specifically to a nucleic acid sequence encoding a constant region of the immunoglobulin lambda light chain. In some embodiments, the first capture sequence binds specifically to a nucleic acid sequence encoding a constant region of the immunoglobulin heavy chain.

Another exemplary embodiment of a spatial methodology for generating immune cell data is depicted in FIG. 16B. In such embodiments, the capture sequence sequence is a homopolymeric sequence, e.g., a polyT sequence. FIG. 16B shows an exemplary poly(A) capture with a poly(T) capture domain. A poly(T) capture domain can capture other analytes, including analytes encoding ABMs within the tissue sample.

In some embodiments, following capture of analytes by capture probes, capture probes can be extended, e.g., via reverse transcription. Second strand synthesis can generate double stranded cDNA products that are spatially barcoded. The double stranded cDNA products, which may comprise ABM encoding sequences and non-ABM related analytes, can be enriched for ABM encoding sequences.

An exemplary enrichment workflow may comprise amplifying the cDNA products (or amplicons thereof) with a first primer that specifically binds to a functional sequence of the first capture probe or reverse complement thereof and a second primer that binds to a nucleic acid sequence encoding a variable region of the ABM expressed by the ABM-expressing cell or reverse complement thereof. In some embodiments, the first primer and the second primer flank the spatial barcode of the first spatially barcoded polynucleotide or amplicon thereof. In some embodiments, the first primer and the second primer flank a J junction, a D junction, and/or a V junction.

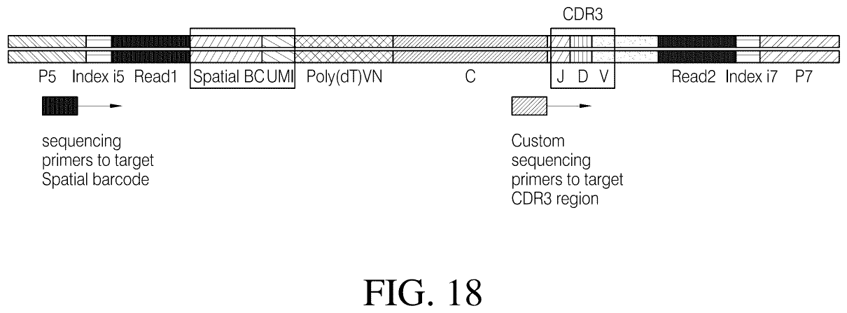

FIG. 17 shows an exemplary analyte enrichment strategy following analyte capture on the array. The portion of the immune cell analyte of interest includes the sequence of the V(D)J region, including CDR sequences. As described herein, a poly(T) capture probe captures an analyte encoding an ABM, an extended capture probe is generated by a reverse transcription reaction, and a second strand is generated. The resulting nucleic acid library can be enriched by the exemplary scheme shown in FIG. 17, where an amplification reaction including a Read 1 primer complementary to the Read 1 sequence of the capture probe and a primer complementary to a portion of the variable region of the immune cell analyte, can enrich the library via PCR. While FIG. 17 depicts a Read 1 primer, it is understood that a primer complementary to other functional sequences, such as other sequencing primer sequences, or sequencer specific flow cell attachment sequences, or portions of such functional sequences, may also be used. While FIG. 17 depicts a polyT capture sequence, it is understood that other capture sequences disclosed herein may be present in library members. The enriched library can be further enriched by nested primers complementary to a portion of the variable region internal (e.g., 5′) to the initial variable region primer for practicing nested PCR.

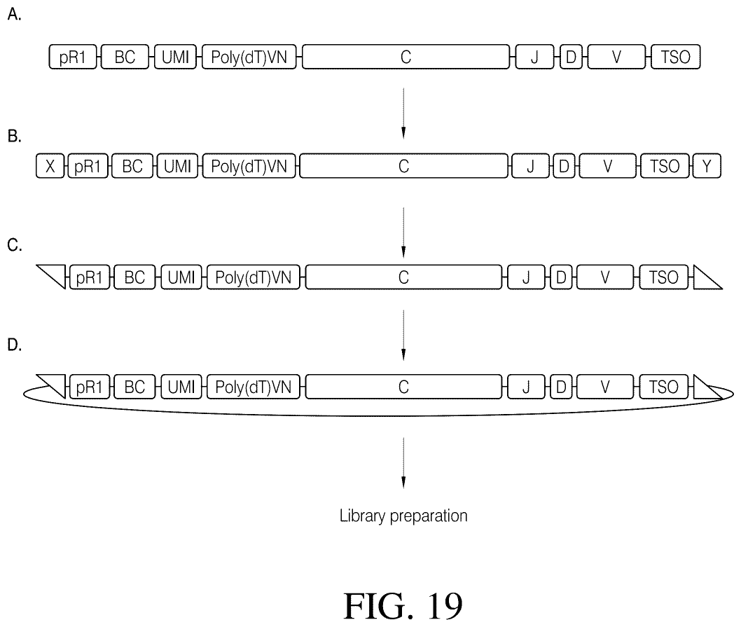

FIG. 18 shows a sequencing strategy with a primer specific complementary to the sequencing flow cell attachment sequence (e.g., P5) and a custom sequencing primer complementary to a portion of the constant region of the analyte. This sequencing strategy targets the constant region to obtain the sequence of the CDR regions, including CDR3, while concurrently or sequentially sequencing the spatial barcode (BC) and/or unique molecular identifier (UMI) of the capture probe. By capturing the sequence of a spatial barcode, UMI and a V(D)J region the receptor is not only determined, but its spatial location and abundance within a cell or tissue is also identified.

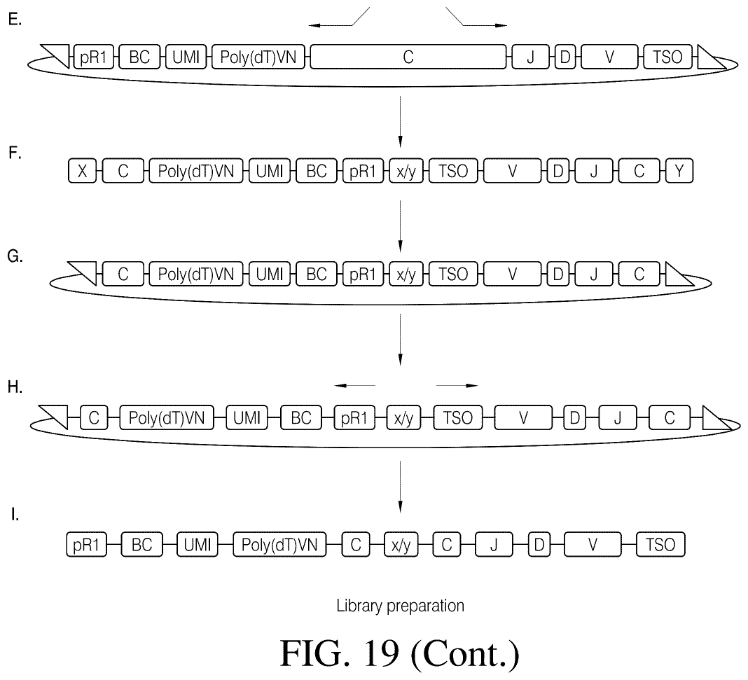

FIG. 19 shows an exemplary nucleic acid library preparation method to remove a portion of an analyte sequence via double circularization of a member of a nucleic acid library. Panel A shows an exemplary member of a nucleic acid library including, in a 5′ to 3′ direction, a first adaptor (e.g., primer sequence R1, pRl (e.g., Read 1)), a barcode (e.g., a spatial barcode or a cell barcode), a unique molecular identifier (UMI), a capture domain (e.g., poly(T) VN sequence), a sequence complementary to an analyte (C, J, D and V), and a second adaptor (e.g., template switching oligonucleotide sequence (TSO)). For purposes of this example an analyte including a constant region (C) and V(D)J sequence are shown, however, the methods described herein can be equally applied to other analyte sequences in a nucleic acid library. Panel B shows the exemplary member of a nucleic acid library where additional sequences can be added to both the 3′ and 5′ ends of the nucleic acid member (shown as a X and Y) via a PCR reaction. The additional sequences added can include a recognition sequence for a restriction enzyme (e.g., restriction endonuclease). The restriction recognition sequence can be for a rare restriction enzyme. The exemplary member of the nucleic acid library shown in Panel B can be digested with a restriction enzyme to generate sticky ends shown in Panel C (shown as triangles) and can be intramolecularly circularized by ligation to generate the circularized member of the nucleic acid library shown in Panel D. The ligation can be performed with a DNA ligase. The ligase can be T4 ligase. A primer pair can be hybridized to a circularized nucleic acid member, where a first primer hybridizes to a 3′ portion of a sequence encoding the constant region (C) and includes a second restriction enzyme (e.g., restriction endonuclease) sequence that is non-complementary to the analyte sequence, and where a second primer hybridized to a 5′ portion of a sequence encoding the constant region (C), and where the second primer includes a second restriction enzyme sequence (Panel E). The first primer and the second primer can generate a linear amplification product (e.g., a first double-stranded nucleic acid product) as shown in Panel F, which includes the second restriction enzyme recognition sequences (shown as X and Y end sequences). The linear amplification product (Panel F) can be digested with a second restriction enzyme to generate sticky ends and can be intramolecularly ligated with a ligase (e.g.. T4 DNA ligase) to generate a second double-stranded circularized nucleic acid product as shown in Panel G. The second double-stranded circularized nucleic product (Panel G) can be amplified with a third primer, pRl, substantially complementary to the first adaptor (e.g., Read 1) sequence and a fourth primer substantially complementary to the second adapter (e.g., TSO) as shown in Panel H to generate a version of the double-stranded member of the nucleic acid library lacking all, or a portion of, the sequence encoding the constant region (C) of the analyte (Panel I). The resulting double-stranded member of the nucleic acid library lacking all or a portion of the constant region can undergo library preparation methods, such as library preparation methods used in single-cell or spatial analyses. For example, the double-stranded member of the nucleic acid library lacking all. or a portion of, the sequence encoding the constant region of the analyte can be fragmented, followed by end repair, a-tailing, adaptor ligation, and/or additional amplification (e.g., PCR). The fragments can then be sequenced using, for example, paired-end sequencing using TruSeq Read 1 and TruSeq Read 2 as sequencing primer sites or any other sequencing method described herein. As such, sequences can be determined from regions more than about 1 kb away from the end of an analyte (e.g., 3′ end) and can link such a sequence to a barcode sequence (e.g., a spatial barcode, a cell barcode) in library preparation methods (e.g., sequencing preparation). For purposes of this example an analyte including a constant region (C) and V(D)J sequences are shown, however, the methods described herein can be equally applied to other analyte sequences in a nucleic acid library.

An exemplary member of a nucleic acid library can be prepared as shown in FIG. 18 to generate a first double-stranded circularized nucleic acid product shown in Panel D of FIG. 18 as previously described.

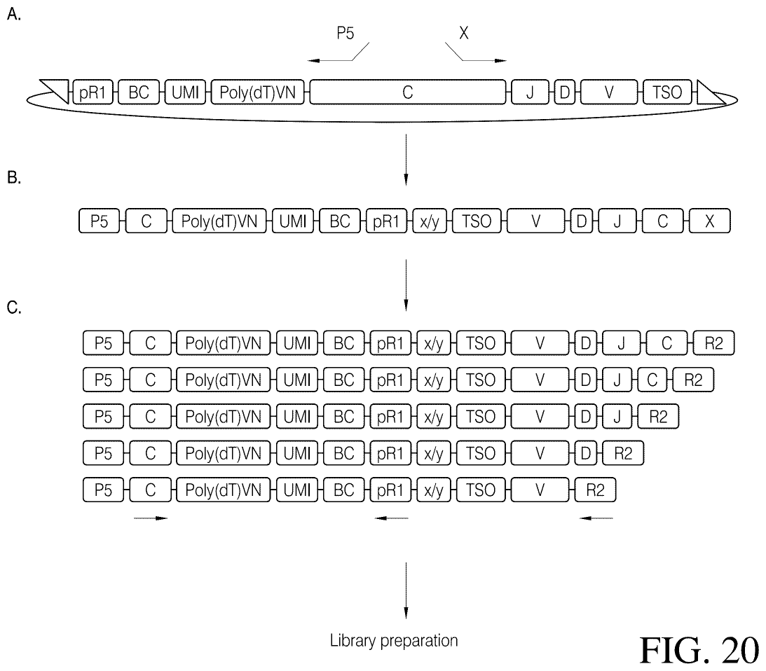

FIG. 20 depicts another exemplary workflow for processing such double-stranded circularized nucleic acid product. A primer pair can be contacted with the double-stranded circularized nucleic acid produce with a first primer that can hybridize to a sequence from a 3′ region of the sequence encoding the constant region of the analyte and a sequence including a first functional domain (e.g., P5). The second primer can hybridize to a sequence from a 5′ region of the sequence encoding the constant region of the analyte, and includes a sequence including a second functional domain (shown as “X”) as shown in Panel A. Amplification of the double-stranded circularized nucleic acid product results in a linear product as shown in Panel B, where all, or a portion of, the constant region (C) is removed. The first functional domain can include a sequencer specific flow cell attachment sequence (e.g., P5). The second functional domain can include an amplification domain such as a primer sequence to amplify the nucleic acid library prior to further sequencing preparation. The resulting double-stranded member of the nucleic acid library lacking all or a portion of the constant region can undergo library preparation methods, such as library preparation methods used in single-cell or spatial analyses. For example, the double-stranded member of the nucleic acid library lacking all, or a portion of, the sequence encoding the constant region of the analyte can be fragmented, followed by end repair, A-tailing, adaptor ligation, and/or amplification (e.g., PCR) (Panel C). The fragments can then be sequenced using, for example, paired-end sequencing using TruSeq Read 1 and TruSeq Read 2 as sequencing primer sites (Panel C, arrows), or any other sequencing method described herein. After library preparation methods described herein, a different sequencing primer for the first adaptor (e.g., Read 1) is used since the orientation of the first adaptor (e.g., Read 1) sequence will be reversed. Accordingly, sequences can be determined from regions more than about 1 kb away from the end of an analyte (e.g., 3′ end) and can link such a sequence to a barcode sequence (e.g., a spatial barcode, a cell barcode) in further library preparation methods (e.g., sequencing preparation). For purposes of this example an analyte including a constant region (C) and V(D)J sequence are shown, however, the methods described herein can be applied to other analyte sequences in a nucleic acid library as well.

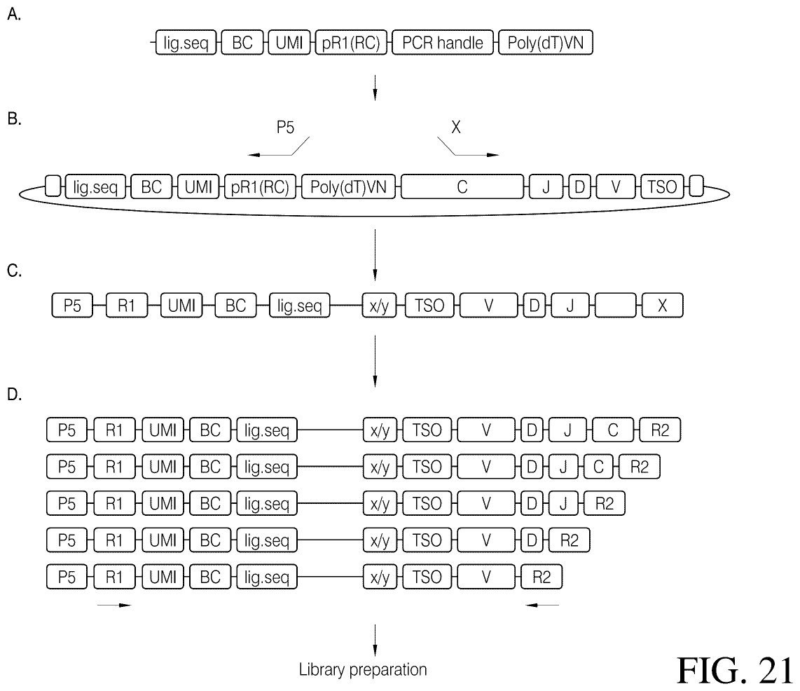

FIG. 21 shows an exemplary nucleic acid library preparation method to remove all or a portion of a constant sequence of an analyte from a member of a nucleic acid library via circularization. Panels A and B shows an exemplary member of a nucleic acid library including, in a 5′ to 3′ direction, a ligation sequence, a barcode sequence, a unique molecular identifier, a reverse complement of a first adaptor (e.g., primer sequence pRl (e.g., Read 1)), a capture domain, a sequence complementary to the captured analyte sequence, and a second adapter (e.g., TSO sequence). The ends of the double-stranded nucleic acid can be ligated together via a ligation reaction where the ligation sequence splints the ligation to generate a circularized double-stranded nucleic acid as shown in Panel B. The circularized double-stranded nucleic acid can be amplified with a pair of primers to generate a linear nucleic acid product lacking all or a portion of the constant region of the analyte (Panels B and C). The first primer can include a sequence substantially complementary to the reverse complement of the first adaptor and a first functional domain. The first functional domain can be a sequencer specific flow cell attachment sequence (e.g., P5). The second primer can include a sequence substantially complementary to a sequence from a 5′ region of the sequence encoding the constant region of the analyte, and a second functional domain. The second functional domain can include an amplification domain such as a primer sequence to amplify the nucleic acid library prior to further sequencing preparation. The resulting double-stranded member of the nucleic acid library lacking all or a portion of the constant region can undergo library preparation methods, such as library preparation methods used in single-cell or spatial analyses. For example, the double-stranded member of the nucleic acid library lacking all, or a portion of, the sequence encoding the constant region of the analyte can be fragmented, followed by end repair, A-tailing, adaptor ligation, and/or amplification (e.g., PCR) (Panel C). The fragments can then be sequenced using, for example, paired-end sequencing using TruSeq Read 1 and TruSeq Read 2 as sequencing primer sites, or any other sequencing method described herein (Panel D). After library preparation methods (e.g., described herein), sequencing primers can be used since the orientation of Read 1 will be in the proper orientation for sequencing primer pRl. Accordingly, sequences can be determined from regions more than about 1 kb away from the end of an analyte (e.g., 3′ end) and can link such a sequence to a barcode sequence (e.g., a spatial barcode, a cell barcode) in further library preparation methods (e.g., sequencing preparation). For purposes of this example an analyte including a constant region (C) and V(D)J sequence are shown, however, the methods described herein can be applied to other analyte sequences in a nucleic acid library as well.

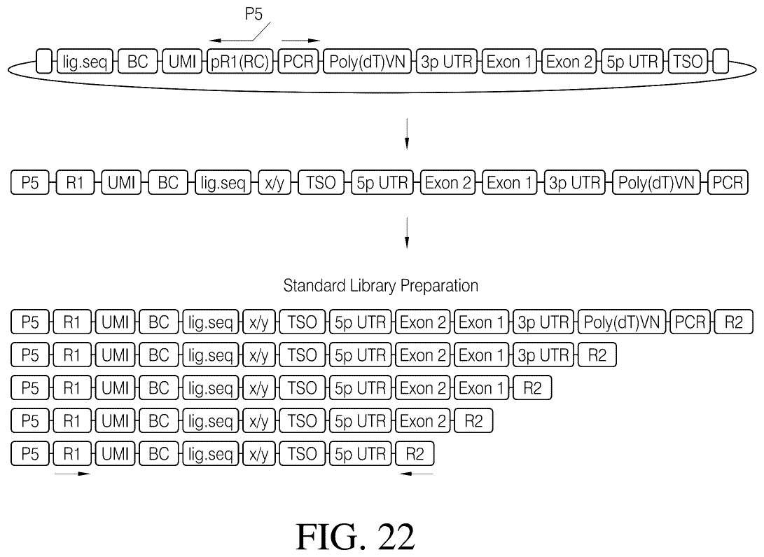

FIG. 22 shows an exemplary nucleic acid library method to reverse the orientation of an analyte sequence in a member of a nucleic acid library. Panel A shows an exemplary member of a nucleic acid library including, in a 5′ to 3′ direction, a ligation sequence, a barcode (e.g., a spatial barcode or a cell barcode), unique molecular identifier, a reverse complement of a first adaptor, an amplification domain, a capture domain, a sequence complementary to an analyte, and a second adapter. The ends of the double-stranded nucleic acid can be ligated together via a ligation reaction where the ligation sequence splints the ligation to generate a circularized double-stranded nucleic acid also shown in Panel A. The circularized double-stranded nucleic acid can be amplified to generate a linearized double-stranded nucleic acid product, where the orientation of the analyte is reversed such that the 5′ sequence (e.g., 5′ UTR) is brought in closer proximity to the barcode (e.g., a spatial barcode or a cell barcode) (Panel B). The first primer includes a sequence substantially complementary to the reverse complement of the first adaptor and a functional domain. The functional domain can be a sequencer specific flow cell attachment sequence (e.g., P5). The second primer includes a sequence substantially complementary to the amplification domain. The resulting double-stranded member of the nucleic acid library including a reversed analyte sequence (e.g., the 5′ end of the analyte sequence is brought in closer proximity to the barcode) can undergo library preparation methods, such as library preparation methods used in single-cell or spatial analyses. For example, the double-stranded member of the nucleic acid library lacking all, or a portion of, the sequence encoding the constant region of the analyte can be fragmented, followed by end repair, A-tailing, adaptor ligation, and/or amplification (e.g., PCR) (Panel C). The fragments can then be sequenced using, for example, paired-end sequencing using TruSeq Read 1 and TruSeq Read 2 as sequencing primer sites, or any other sequencing method described herein. Accordingly, sequences from the 5′ end of an analyte will be included in sequencing libraries (e.g., paired end sequencing libraries). Any type of analyte sequence in a nucleic acid library can be prepared by the methods described in this Example (e.g., reversed).

### IV. Selecting Cells of Interest Based on Immune Cell Data Visualization

Various method and system embodiments described herein enable improved methods to select cell candidates with therapeutic potential from a clonotype based on visualization of immune cell data. For example, visualization schemes and methods can be used for displaying the location of specific mutations, the type of amino acids present, the abundance (e.g., quantities or frequency) and location of protein motifs that may pose developability challenges for therapeutic purposes and for displaying evolutionary distances of various exact subclonotypes from a reference sequence. In one or more embodiments, the visualization methods and systems described herein may be used to develop therapeutics for SARS-CoV-2. The visualization methods and systems described herein may be used to identify or otherwise select anti-SARS-CoV-2 S antibodies and antigen-binding fragments thereof and to provide therapeutic methods of using such antibodies and fragments for treating viral infections based on SARS-CoV-2. For example, one or more of the embodiments described herein provide visualization schemas that enable faster and more efficient isolation of antibodies or antigen-binding fragments thereof, that bind specifically to a spike (S) protein of severe acute respiratory syndrome coronavirus 2 (SARS-CoV-2).

In various embodiments, current visualization schemes achieve horizontal and vertical compactions. As there are only so many letters (represented bases or amino acids) that can be view in a row before the GUI becomes visually overwhelming, the letters/positions that are variable are displayed within a clonotype, hence, horizontal compaction. Since each exact subclonotype is comprised of a set of one or more cells, inclusion of additional data to display, such as gene expression, antigen capture, surface protein/antibody capture, etc. could be used to display this data for each cell rather than a single line with summary statistics for an exact subclonotype in vertical compaction.

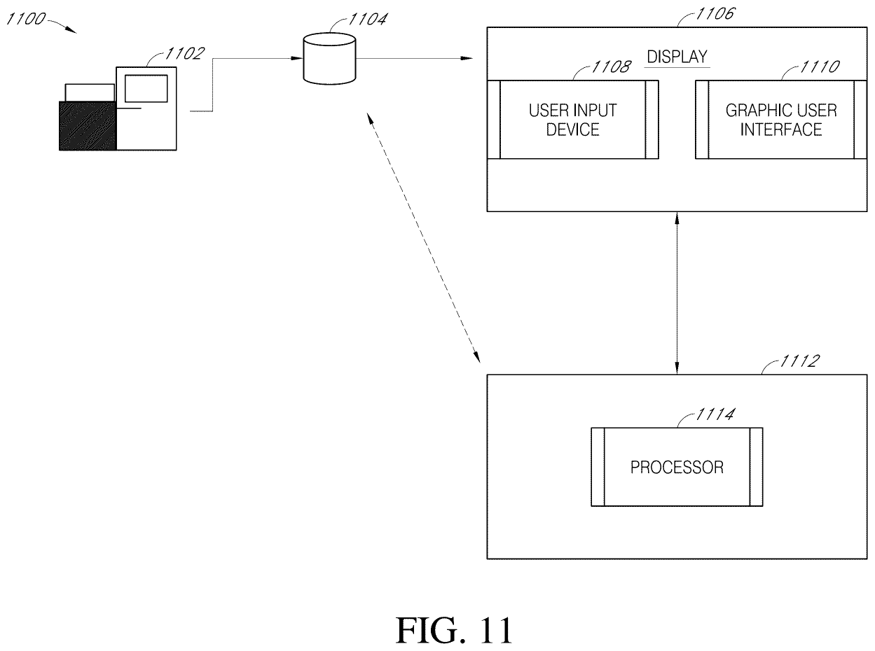

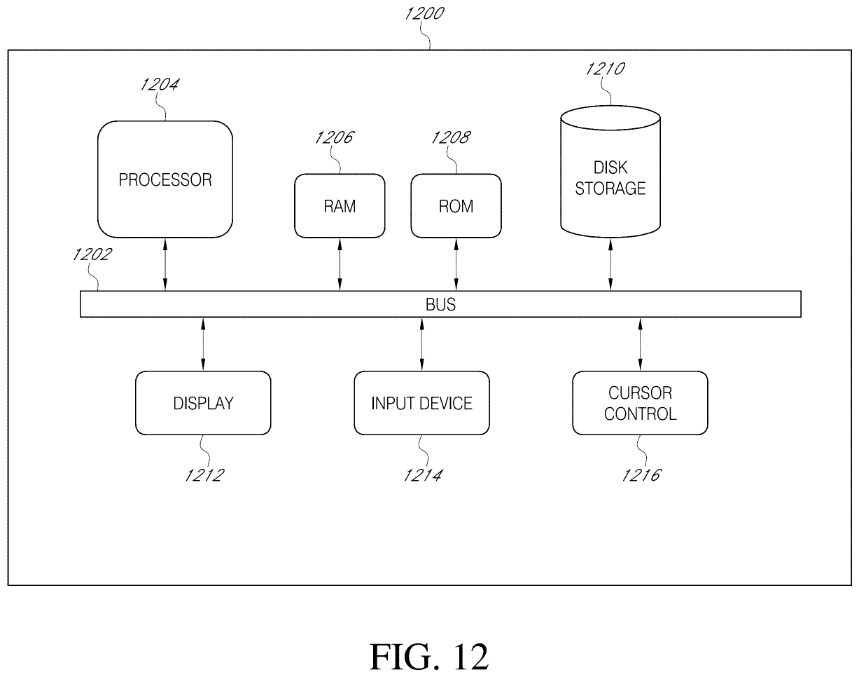

The embodiments described herein provide a clonotyping information visualizer, which may be implemented using, for example, without limitation, computer system 1100 in FIG. 11 or computer system 1200 in FIG. 12. The clonotyping information visualizer, which may be implemented using software, hardware, firmware, or a combination thereof, may generate the visualization of sequences (e.g., V(D)J sequences) that can be rendered for display on a display system (e.g., display system 1106 in FIG. 11 or display 1212 in FIG. 12). The visualization that is generated uses one or more visualization schemas to provide information about a sequence (e.g., V(D)J sequence).

Unlike currently available systems for visualization V(D)J sequences, the visualization schema(s) used by the clonotyping information visualizer described herein enable a user to quickly identify or recognize which nucleotides, codons, and/or amino acids are different from the germline alleles present in a sample. The visualization schema(s) also highlights changes that have originated from somatic hypermutation and from possible molecular biology artifacts and/or errors in V(D)J references. The visualization schema(s) also enables easy and efficient visualization with side-by-side CSV (comma-separated values) (and/or FASTA and phylogenetic tree file formats) output. This information may be displayed on a display system in a manner that enables at least a portion of the information or all of this information (e.g., all of the CSV output) to be copied and pasted into a variety of tools. The visualization schema(s) separates the clonotypes from each other and helps prevent users from accidentally copying the wrong sequences.

The clonotyping information visualizer may receive user input data associated with clonotypes and/or barcodes (e.g., cell barcodes or spatial barcodes) within the same display as the visualization of the sequence, giving users a side-by-side rapid approach to combine their functional data and explore new hypotheses without having to write any additional code. The visualization generated by the clonotyping information visualizer is a compact display that enables viewing without having to scroll side-to-side through multiple spreadsheets, and without requiring users to write a macro (e.g., VBA macro) for a spreadsheet program, such as Excel. Existing spreadsheet systems are unable to provide the same level of detailed information about sequences using, for example, color, without requiring entirely separate plotting code and architecture in other languages (e.g., Python, R, Julia etc.).

The clonotyping information visualizer may be capable of processing hundreds of thousands of clonotypes from, for example, over 1 million cells in a matter of minutes. For example, the clonotyping information visualizer may be capable of processing hundreds of thousands of cells from approximately 1.3 million cells in about 7 minutes using 16-30 GB of RAM. Other existing tools do not have the same efficiency as the clonotyping information visualizer described herein and, in some instances, have been known to fail due to out-of-memory errors on a server with 72 cores, occupying and then overflowing 384 GB of RAM. As one example, an existing tool may take about 65 minutes or more when parallelized to analyze about 300,000 sequences. However, the clonotyping information visualizer may take about 11 minutes to analyze the sequences of about 1.3 million cells, while also being capable of providing other information, such as the FR and CDR annotations, germline inference, other types of information, or a combination thereof.

The clonotyping information visualizer may be configured to only use a defined number of cores and threads, unlike certain existing software. In one or more embodiments, when the clonotyping information visualizer is combining multiple data modalities from a sample, the clonotyping information visualizer can check to see whether the data can be compressed and reformatted to accelerate processing and thereby use less RAM and fewer threads on re-analysis.

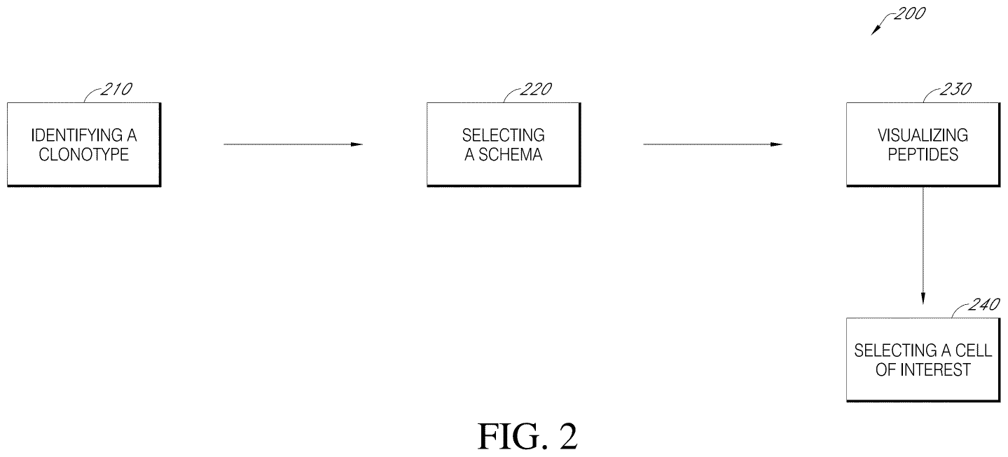

FIG. 2 illustrates non-limiting exemplary embodiments of a general schematic workflow for selecting cell candidates with therapeutic potential from a clonotype in accordance with various embodiments. The workflow 200 can comprise, at step 210, identifying a clonotype using single cell data. An immune cell receptor clonotype is a group of T or B cells sharing a fully rearranged common ancestor immune cell; a clonotype contains one or more exact subclonotypes, each of which bears a unique nucleotide sequence. The combination of nucleotide sequences for the surface-expressed immune receptor heterodimer would define the immune cell receptor clonotype.

Clonotyping is a process to identify which cells share a fully rearranged common ancestral cell given the unique nucleotide full-length or CDR3 sequences of one or more immune cell receptor chains belonging to one or more immune cells. This can involve template-switching for generating full-length immune cell receptor sequences that are amplified using nested PCR reactions targeting barcode regions and each of the constant region genes, followed by nucleotide sequencing of the amplicon, or alternatively, and additionally, identification and comparison of unique CDR3 sequences from single cell data sets.

Single cell data sets can include a data set of immune cell receptors or fragments thereof from single cells. For example, the single cell data set comprises a data set of a B cell receptor, a T cell receptor, a single-chain variable fragment (ScFv), an antigen-binding fragment (Fab), or a combination thereof. Single cell data can be obtained by any single cell sequencing methods, such as exemplified in FIG. 1.

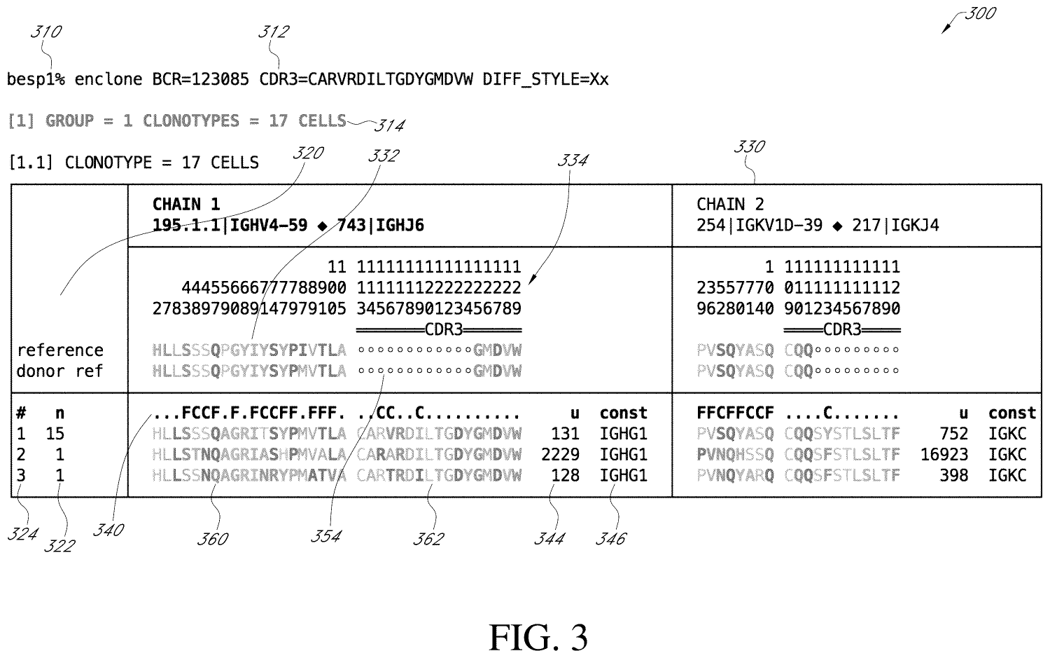

The workflow 200 can comprise, at step 220, selecting a schema from several schemas as exemplified in FIGS. 3-8 or a phylogenetic tree. At least a portion of workflow 200 and the visualization schemas described in FIGS. 3-8 may be implemented using the clonotyping information visualizer, which may be implemented using, for example, without limitation, computer system 1100 in FIG. 11 or computer system 1200 in FIG. 12. The schema is selected to visualize amino acids of mutations or protein motifs in the clonotype group based on at least one of positions or chemical identity of the selected amino acids. For example, the schema may be selected to label (e.g., highlight) locations and numbers of mutations or protein motifs. The schema can also be a schema for generating a phylogenetic tree that can be represented as nucleotide distances between exact subclonotypes. The phylogenetic tree can be used to visualize and compare antibody sequences and can also be combined with other information present in the graphic representation.

The workflow 200 can comprise, at step 230, visualizing the selected amino acids in a graphic representation according to the schema as exemplified in FIGS. 3-8. For example, the graphic representation can be a graphic user interface (GUI). The graphic representation can include an alignment of several amino acid sequences in the clonotype and include labeling of mutations and protein motifs in amino acid sequences to provide information for therapeutic development.

The workflow 200 can comprise, at least step 240, selecting one or more cells of interest. The cell of interest can be selected based on a pre-defined criterion using the graphic representation. For example, the pre-defined criterion may include a rank of the number of mutations in a defined region such as CDRs, or a minimum or maximum number of mutations in defined locations (e.g., FWR or CDR) as determined by known immune receptors with therapeutic significance or a known nucleotide distance between exact subclonotypes or from a reference sequence.

One way of selecting cells of interest is to choose cells from the phylogeny that have both a specific constant region (conferring unique functional activity) and which are furthest from the unmutated common ancestor (longest distance along the branches). Distance from donor reference is the same thing as lower % identity to germline/higher occurrence of somatic hypermutations. Choosing cell clones with the highest number of mutations in the CDRs is another selection strategy, which can be coupled with the number of nonsynonymous mutations/codons relative to the reference sequence.

Additionally, or alternatively, choosing members of the phylogeny that occur within broader branches is helpful in selecting for clones with higher affinity to the antigen of interest. This can be achieved using a Bayesian phylogenetic hidden Markov model for B cell receptor sequence analysis, using B cell receptor lineage structures to predict affinity, or using Likelihood-Based Inference of B Cell Clonal Families, or a combination thereof (Ralph DK, Matsen FA IV (2016) Likelihood-Based Inference of B Cell Clonal Families. PLoS Comput Biol 12(10): e1005086, available through https://doi.org/10.1371/journal.pcbi.1005086; Dhar A, Ralph DK, Minin VN, Matsen FA IV (2020) A Bayesian phylogenetic hidden Markov model for B cell receptor sequence analysis. PLoS Comput Biol 16(8): e1008030, available through https://doi.org/10.1371/journal.pcbi.1008030; Ralph DK, Matsen FA IV (2020) Using B cell receptor lineage structures to predict affinity. PLOS Computational Biology 16(11): e1008391, available through https://doi.org/10. 1371/j ournal.pcbi.1008391).

The view and schemas presented in this disclosure allow straightforward presentation of the differences within a clonotype relative to a reference sequence (such as a universal reference, a donor-derived germline reference, or a combination thereof) or other exact subclonotypes. FIGS. 3-8 provide exemplary visualization schemas to visualize identified clonotypes and the differences.

Additionally, and alternatively, a phylogenetic tree can be a schema to visualize and compare sequences. A phylogenetic tree can be combined with the other information present in the display for ease of visualization and legibility. The phylogenetic tree is one of many possible implementations of the result from a calculation comparing the nucleotide distances between exact subclonotypes. Distances between exact subclonotypes can be calculated using a number of different distance metrics (Jaro-Winkler, cosine similarity, Levenshtein/Damerau-Levenshtein/optimal string alignment, Hamming, q-gram, longest common substring, and Jaccard distances), and a number of phylogenetic tree algorithms (maximum parsimony, maximum likelihood estimation, UPGMA, Bayesian maximum likelihood estimation).

Additionally, and alternatively, comparing the nucleotide distances between exact subclonotypes also enables the grouping of cells into clonotypes—the identification of immune cells which share a set of common ancestors. Clonotypes frequently share antigen specificities, and sublineages within a clonotype may have differing fine specificities or cross-reactivities. This can also be used to find similar immune cell receptors in other datasets to identify new immune receptors with diagnostic or therapeutic potential.

Schemas as exemplified in FIGS. 3-6 display the marking of framework and CDR mutations, which can enable users to easily tell whether or not a mutation of interest in one or more exact subclonotypes may occur in an antigen-binding region of the immune cell receptor, and to identify common positions or amino acids within a clonotype that are associated with antigen binding (or lack thereof).

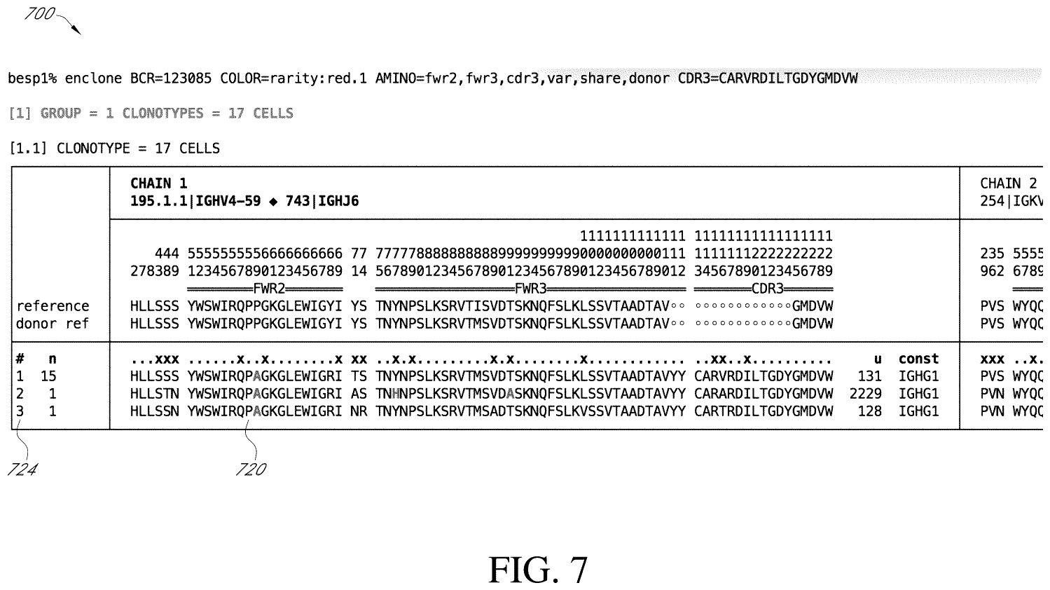

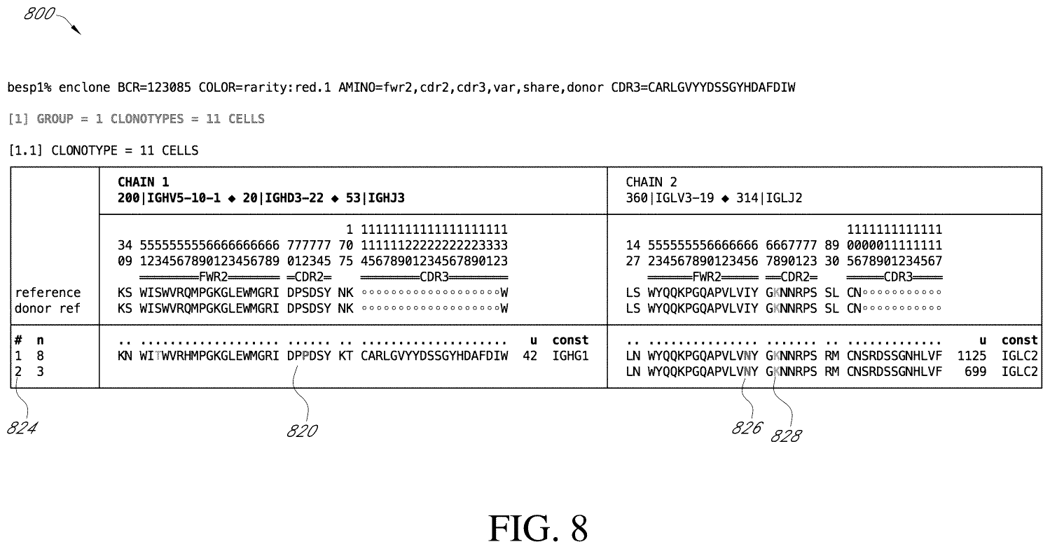

Schemas as exemplified in FIGS. 7-8 include the display of different properties of the sequences within a clonotype (including, but not limited to, codon usage, amino acid property, developability flags) in a compact and digestible space, and the ability to easily add user-specified information in a table-like format.

Separately, being able to identify whether or not an amino acid that induces possible developability issues occurs frequently within a set of reference sequences or reference species provides information as to the “effect size” or actual likelihood of that amino acid or motif posing a problem in therapeutic development (e.g., if it occurs commonly in the germline it is structurally conserved and likely not a real problem). Additional filters can be added to a command producing this display to sort by the number of mutations in the sequence, the number of FWR mutations or CDR mutations in the sequence, etc.

It should be noted that many details about the display features, fields, parameters, customizations, etc. are discussed elsewhere can be applied to this discussion of the visualization schema of FIGS. 3-8. It should be understood that while many of these details are discussed in different sections, the display features, fields, parameters, customizations, etc., and the associated descriptions are relevant to all embodiments herein and can be implemented in any combination per user need. Referring to FIG. 3, an example visualization schema 300 of identified clonotypes

is provided, in accordance with various embodiments. Visualization schema 300 can include a command line 310 configured to accept a user input, in accordance with various embodiments. This user input can include, for example, one or more user-selected parameters 312 for customizing the output in visualization schema 300. As will be discussed below, specifying data sets can be done various ways including, for example, on the command line 310 or via a supplementary metadata file. In the example visualization schema 300, the command line 310 includes BCR and CDR3 parameters 312. Based on this example command line entry 310, the output of the visualization schema 300 could exhibit all cells in a particular identified clonotype, in which at least one antibody chain in the cells has the given CDR3 sequence. The output can be in a compressed view (e.g., streamlined visualization of query results to include useful information for specific analytical purposes).

Visualization schema 300 can include a clonotype grouping statement 314, which can include information such as, for example, the number of clonotype groups (one in FIG. 3), the number of clonotypes in the noted group (one in FIG. 3), and the number of cells in the noted clonotype (“17 cells” in FIG. 3). Clonotypes can be grouped into similar families sharing putatively similar function, with the grouping done automatically or via user-specified filters. These filters can include grouping clonotypes based on V gene, similarity across the CDR3/junction sequence or the full-length heavy and/or light chains, reporting of singleton chains matching higher-frequency subclonotypes, detection and identification of indels within exact subclonotypes, quantity of expressed gene, quantity of detected antigen, and more. In accordance with various embodiments, a display can conceptually distinguish between clonotypes (e.g., as evolutionary families) and clonotype groups (e.g., as functional families).

As discussed above, visualization schema 300 can also include a subclonotype listing frame 320 for an immune cell clonotype, in accordance with various embodiments. The exact subclonotypes can share identical V(D)J transcripts. The listing of exact subclonotypes can include a number of cells 322 associated with each exact subclonotype as listed in 324. Each line of 324 can be configured to represent an exact subclonotype, a set of cells having identical V(D)J transcripts.

Further, the subclonotype listing frame 320 can include subclonotype information selected from the group consisting of gene expression, Hamming distance, antibody, and combinations thereof. The gene expression subclonotype information can be selected from the group consisting of median gene expression, maximum gene expression, mean gene expression, and combinations thereof. The gene expression subclonotype information can be reported as a unique molecular identifier (UMI) count, such as a median, maximum, mean UMI count. Median, maximum, mean, and similar summary statistics thereof can also be used in accordance with various embodiments to visualize and report the aforementioned features in addition to gene expression. Those with ordinary skill in the art would recognize that there are many additional such features that could be reported such as a percentage of a given set of features within a single cell and other user-provided annotations for a set of single cells, such as manual annotation or description of information relevant to one or more exact subclonotypes, as specified in a variety of file formats.

As discussed above, visualization schema 300 can also include a listing of one or more frames 330, in accordance with various embodiments. Frames 330 can include information about chains common to each member of the immune cell clonotype population. Frames 330 can include an amino acid or nucleotide sequence for the variable and constant regions of each exact subclonotype. Visualization schema 300 can generally output one, two, three, four, five, six, seven, or more frames 330. FIGS. 3-8 illustrate two textual frames for chain 1 and chain 2.

Frames 330 can display many different types of information and can also be readily configured via user instruction to display those many different types of information in virtually any combination. Frames 330 can show positional information 334 for each member of the amino acid sequence. Numbered columns of the position information 334 show the position of a particular amino acid. For example, reading vertically, the third column of the first chain shows a “43”, which can represent amino acid at position 43 in the first chain (where position 0 is the start codon). The position information 334 can be displayed horizontally or in any other ways known in the art.

Frames 330 can include a listing of amino acid or nucleotide differences 340 between sequences of each exact subclonotype 324 of the clonotype population. A symbol, such as “C” or “F” is shown in FIG. 1 at a column position where variation occurs within the clonotype or where a somatic hypermutation occurs. These notations can indicate the raw evolutionary history of the clonotype and indicated the positions containing information relevant to calculating an antibody phylogeny. For example, these differences 340 can represent whether detected mutations in an antibody or a T cell receptor (TCR) clonotype occurs in complementarity determining regions (CDR) as represented as “R” or in surrounding framework regions (FWR) as represented as “F.”

This visualization (i.e., display) can be functional and helpful in selection of a cell of interest from the clonotype group based on a pre-defined criterion using the graphic representation in the visualization schema 400. In particular, the visual format allows users to prioritize and sort antibodies and TCRs based on the number of FWR and CDR mutations and select cells of interest based on FWR and CDR mutations. For example, ranking cells based on the number of mutations in the CDRs and choosing cell clones with the highest number of mutations (or a predefined rank number, e.g., top 1, 2, 3, 4, 5, 6, 7, 8, 9, 10% or any intermediate ranges or numbers) in the CDRs can be a selection strategy, which can be coupled with the number of nonsynonymous mutations/codons relative to the reference sequence.

Amino acids can be colored in a fashion dependent on which detected codon represents a given amino acid. Moreover, synonymous changes can be displayed using different colors to display variability between exact subclonotypes with different nucleotide sequences at variable positions but identical amino acid sequences. A synonymous mutation is a change in the DNA sequence that codes for amino acids in a protein sequence but does not change the encoded amino acid. Due to the redundancy of the genetic code (multiple codons code for the same amino acid), these changes usually occur in the third position of a codon. On frames 330, amino acids are displayed as being associated with a specific exact subclonotype if the displayed amino acid 360 differs from the universal reference sequence or the displayed amino acid 362 is also in the CDR provided (see “CDR3=CARVRDILTGDYGMDVW” in the parameters 312).

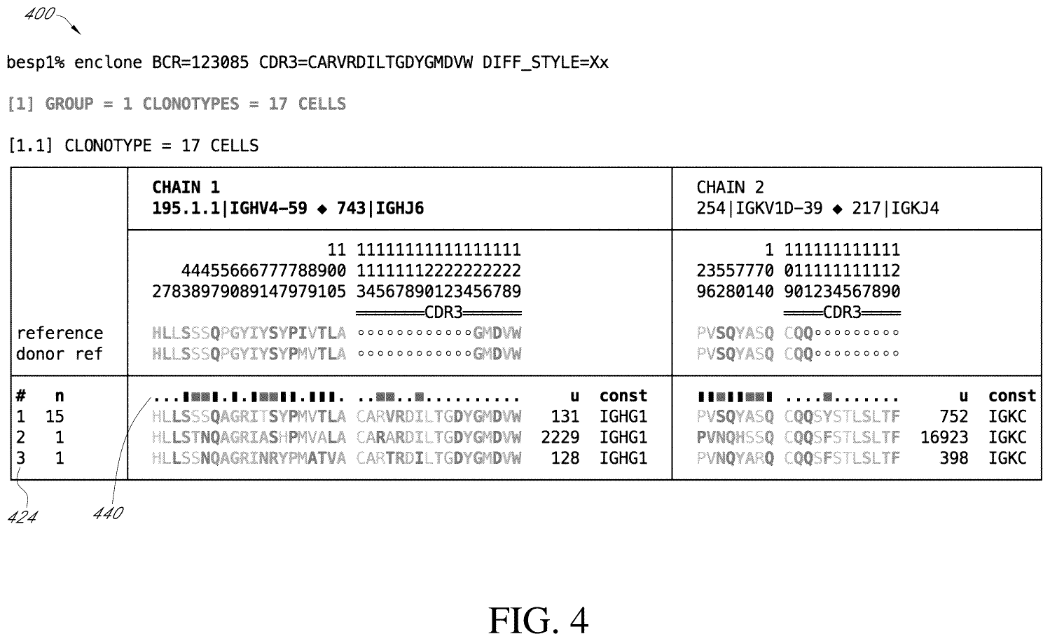

Frames 330 can show a comparison of at least one reference sequence 332 to each sequence associated with exact subclonotypes 324. The at least one reference sequence can include a reference sequence listing selected from the group consisting of a universal reference sequence, a donor reference sequence, and combinations thereof. A reference sequence is a sequence found in a public database and often the single sequence for a given genomic segment that is found in the reference sequence for the given species. A donor reference sequence is a modified version of this universal reference sequence that has mutations introduced and that are believed to have arisen in the germline sequence of the donor. The donor reference sequence is derived using data from the immune receptor dataset, where V segments (in various embodiments, also D and J segments) from multiple cells are used to impute shared mutations between different clonotypes, where the shared mutations represent the germline mutations found in a given V, D, or J gene of a donor. These mutations are found by observing mutations that are common to several different clonotypes sharing a given segment. FIG. 3, for example, displays both reference sequences and donor reference sequences, as does FIGS. 4-8. The symbol [°] 354 represents gaps in the recombined region where display of the reference sequence would be unhelpful, specifically where it is too difficult to confidently identify where the reference sequence ends and where the junction region begins.

Frames 330 can display germline changes as well, which are allelic variations distinct from variations caused by somatic hypermutation. For example, the notation “195.1.1” for chain 1 on FIG. 3 can mean that this V reference sequence is an alternate allele derived from a reference sequence (contig in the reference file) numbered 195, which is from donor 1 (hence “195.1”) and is the alternate allele 1 for that donor (hence “195.1.1”).

For each exact subclonotype, the textual frames 330 can provide chain-specific subclonotype information selected from the group consisting of V(D)J unique molecular identifier (UMI) count, V(D)J read count, constant region name, complementarity-determining region (CDR) sequence, constant sequence length, 5′UTR sequence length, differences from a universal reference constant region, differences from the 5′UTR sequence, base differences between exact subclonotypes, and combinations thereof. Referring to FIG. 3, for example, the provided chain-specific subclonotype information includes median UMI read count 344 for each exact clonotype and constant region name 346 associated with each chain in the given exact subclonotype. Median, maximum, mean, and similar summary statistics thereof can also be used in accordance with various embodiments to visualize and report the aforementioned features in addition to exact subclonotypes. Those knowledgeable in the art recognize that there are many additional features that could be reported such as percentage of a given set of features within a single cell and other user-provided annotations for a set of single cells such as manual annotation or description of information relevant to one or more exact subclonotypes, as specified in a variety of file formats.

Regarding UMI, for a given chain, a given cell contains a certain number of mRNA molecules representing that chain. Each of those that is reverse transcribed is tagged with a UMI, and the total number of UMIs that is found is thus a downward-biased estimate, for a given chain in a given cell, of the number of mRNA molecules that were present. For a given chain in a given exact subclonotype, the median of the UMI counts for all the cells in the exact subclonotype (for the given chain) is recorded and displayed. In accordance with various embodiments, it should be noted that, at times, some chains are missing from exact clonotypes.

For more detail regarding customization of visualizations, in accordance with various embodiments, refer to the Additional Features section below for detailed discussion. It should be noted that the various parameters, variables, fields, values, filters, etc. discussed in detail herein are independent and interchangeable in any contemplated fashion or combination. Moreover, the various parameters, variables, fields, values, filters, etc. discussed in detail herein are applicable to any and all the various embodiments discussed or contemplated herein.

Referring to FIG. 4, another example visualization schema 400 of identified clonotypes is provided, in accordance with various embodiments. This visualization schema 400 shares many similar characteristics to visualization schema 300 of FIG. 3 except as noted below. Of note is a listing of amino acid or nucleotide differences 440 between sequences of each exact subclonotype 424 of the clonotype population. These differences 440 can represent whether detected mutations in an antibody or a T-Cell receptor (TCR) occurs in complementarity determining regions (CDR) as represented by a first symbol or in surrounding framework regions (FWR) as represented as a second symbol. The first symbol may be a first shape, a first color, or a combination thereof, such as a red square box; the second symbol may be a second shape, a second color, or combination thereof, such as a narrow rectangular box. This visualization (i.e., display) can be functional and helpful in selection of a cell of interest from the clonotype group based on a pre-defined criterion using the graphic representation in the visualization schema 400. In particular, the visual format allows for easy visual comparison of the location and quantity of CDR and FWR mutations, and to prioritize and sort antibodies and TCRs based on the location and number of CDR and FWR mutations.

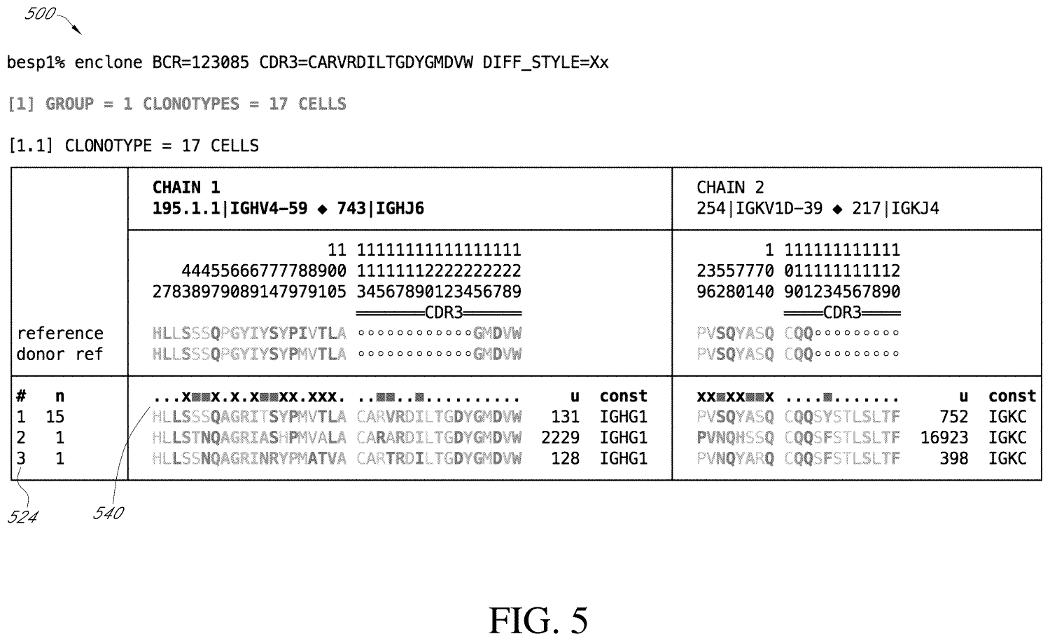

Referring to FIG. 5, another example visualization schema 500 of identified clonotypes is provided, in accordance with various embodiments. This visualization schema 500 shares many similar characteristics to visualization schema 300 of FIG. 3 except as noted below. Of note is a listing of amino acid or nucleotide differences 540 between sequences of each exact subclonotype 524 of the clonotype population. These differences 540 can represent whether detected mutations in an antibody or a T-Cell receptor (TCR) occurs in complementarity determining regions (CDR) as represented by a non-character symbol or in surrounding framework regions (FWR) as represented as a character symbol. The non-character symbol may be a selected shape, a selected color, or a combination thereof, such as a red square box; the character symbol may be a selected number or a selected letter character, such as a letter “x.” This visualization (i.e., display) can be functional and helpful in selection of a cell of interest from the clonotype group based on a pre-defined criterion using the graphic representation in the visualization schema 400. In particular, the visual format allows for easy visual comparison of the location and quantity of CDR and FWR mutations, and to prioritize and sort antibodies and TCRs based on the location and number of CDR and FWR mutations.

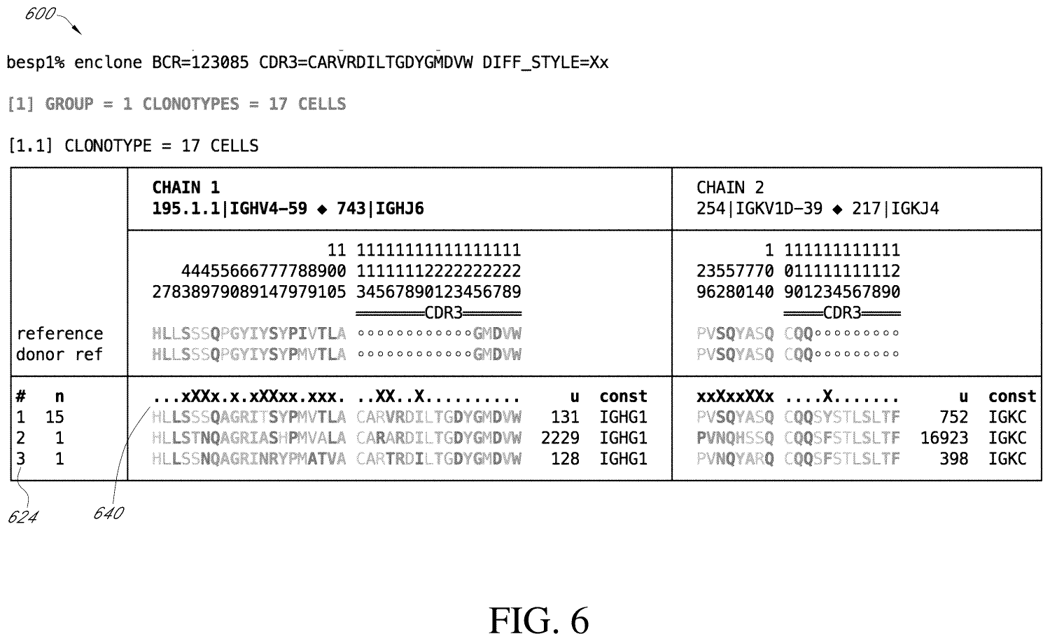

Referring to FIG. 6, another example visualization schema 600 of identified clonotypes is provided, in accordance with various embodiments. This visualization schema 600 shares many similar characteristics to visualization schema 300 of FIG. 3 except as noted below. Of note is a listing of amino acid or nucleotide differences 640 between sequences of each exact subclonotype 624 of the clonotype population. These differences 640 can represent whether detected mutations in an antibody or a T-Cell receptor (TCR) occurs in complementarity determining regions (CDR) as represented by a first character symbol or in surrounding framework regions (FWR) as represented as a second character symbol. The first character symbol may be a selected number or letter character, such as an upper-case X; the second character symbol may be a different selected number or letter character, such as a lowercase x. This visualization (i.e., display) can be functional and helpful in selection of a cell of interest from the clonotype group based on a pre-defined criterion using the graphic representation in the visualization schema 400. In particular, the visual format allows for easy visual comparison of the location and quantity of CDR and FWR mutations, and to prioritize and sort antibodies and TCRs based on the location and number of CDR and FWR mutations.

Referring to FIG. 7, another example visualization schema 700 of identified clonotypes is provided, in accordance with various embodiments. This visualization schema 700 shares many similar characteristics to visualization schema 300 of FIG. 3 except as noted below. Of note is a way of display amino acids sequences 720 of each exact subclonotype 724 of the clonotype population. Amino acids that occur with a frequency meeting a predetermined or user-selected threshold can be identified and displayed, e.g., highlighted with color, underlining, bold, or any known visual formatting. The threshold can be selected or adjusted by a user to highlight amino acids occurring in less than, greater than, or equal to a frequency at which the particular amino acid is occurring in a corresponding reference sequence (e.g., a single reference sequence or a reference set of sequences) at the same position. For example, the threshold is less than 1% frequency of the corresponding reference sequence at the same position. The amino acids occurring in less than 1% of the reference sequence can be identified and labeled (e.g., highlighted) in this example as shown in FIG. 7. This visualization (e.g., display) can be functional and helpful in selection of a cell of interest from the clonotype group based on a pre-defined criterion using the graphic representation in the visualization schema 700. In particular*, the visual format allows users to facilitate analysis of how conserved an amino acid appearing at certain position should be and to rapidly identify suspicious amino acids.

Referring to FIG. 8, another example visualization schema 800 of identified clonotypes is provided, in accordance with various embodiments. This visualization schema 700 shares many similar characteristics to visualization schema 700 of FIG. 7 except as noted below. Of note is a way of display amino acids sequences 820 of each exact subclonotype 824 of the clonotype population. Amino acids that occur with a frequency meeting a first predetermined or user-selected threshold can be identified and displayed by a first label 826 (e.g., a color, underlining, bold, or any known visual formatting), and amino acids that occur with a frequency meeting a second predetermined or user-selected threshold can be identified and displayed by a second label 828 (e.g., a different color, underlining, bold, or any known visual formatting). For example, amino acids that occur in less than 1% of a corresponding reference sequence is labeled with a first color, such as blue, and amino acids that do not occur in any of the reference sequences are labeled with a second color, such as red. This visualization (i.e., display) can be functional and helpful in selection of a cell of interest from the clonotype group based on a pre-defined criterion using the graphic representation in the visualization schema 800. In particular, the visual format allows users to facilitate analysis of how conserved an amino acid appearing at certain position should be and to rapidly identify suspicious amino acids.

Additionally, and alternatively, these visualization schemas can be used to color and mark protein motifs that encode post-translational modifications so that users can visualize whether or not and where mutations that arc undesirable for drug development occur within an antibody or T cell receptor sequence. This visualization (i.e., display) can be functional and helpful in selection of a cell of interest from the clonotype group based on a pre-defined criterion using the graphic representation.

Additionally, and alternatively, these visualization schemas can be used to highlight regions or amino acids or nucleotides of an immune cell receptor with poor read or UMI support, and combined with filters that show one or more FWR and CDR sequences, b. highlight structurally conserved regions by setting a high % conservation threshold, c. highlight regions that arc newly identified to bind antigen(s) or known sequences and positions that bind antigen(s). d. highlight recombination signal sequences in an immune cell receptor, e. highlight sequences or chains of an immune cell receptor that are frequently (at some % threshold) associated with the presence of an additional chain of the same type

In accordance with various embodiments, these visualization schemes (e.g., displays) can also be vertically expanded to display the same information at the per-barcode level in place of the per-exact subclonotype level. In accordance with various embodiments, these visualizations can be customized to group cells based on sample-level, clonotype-level, or barcode-level information (e.g., how many cells in an exact subclonotype are from a given time point or a given donor, etc.).

In accordance with various embodiments, a GUI is provided for displaying immune cell clonotyping information. The GUI can include a listing of exact subclonotypes of an immune cell clonotype, wherein the exact subclonotypes share identical V(D)J transcripts, and wherein the listing of exact subclonotypes includes a number of cells associated with each exact subclonotype. The GUI can further include a listing of one or more textual frames with information about chains common to each member of the immune cell clonotype, wherein the textual frame contains an amino acid sequence for the variable and constant regions of each exact subclonotype. The GUI can further include a positional information for selected amino acids of the amino acid sequence, wherein the selected amino acids are selected based on at least one of positions or chemical identity of the selected amino acids.

In accordance with various embodiments the nucleotide sequences and accompanying positional information for the variable and constant regions of each exact subclonotype can be displayed in place of or in parallel to the amino acid sequences for these regions.

In accordance with various embodiments, the listing of one or more textual frames can comprise two or more textual frames. In accordance with various embodiments, the listing of one or more textual frames can comprise two textual frames. In accordance with various embodiments, the listing of one or more textual frames can comprise three textual frames. It should be understood, however, that the listing of textual frames can include any number of textual frames as long as it can be rendered on a computer display in a manner that can be navigated by a user.

In accordance with various embodiments, the listing of one or more textual frames can include a comparison of at least one reference sequence to an exact subclonotype. The at least one reference sequence can include a reference sequence listing selected from the group consisting of a universal reference sequence or user-supplied reference, a donor reference sequence, and combinations thereof. In accordance with various embodiments, the listing of one or more textual frames includes a listing of amino acid differences between each exact subclonotype within a clonotype. In accordance with various embodiments, the listing of one or more textual frames includes a listing of nucleotide differences between each exact subclonotype within a clonotype.

In accordance with various embodiments, the listing of exact subclonotypes includes subclonotype information selected from the group consisting of gene expression, Hamming distance, Levenshtein distance or similar edit distance, antibody counts, antigen counts, CRISPR guide or directly captured feature counts, and combinations thereof. The gene expression subclonotype information can be selected from the group consisting of median gene expression, maximum gene expression, mean gene expression, and combinations thereof. The gene expression subclonotype information can be reported as a UMI count for each cell belonging to a given exact subclonotype; the features listed above can also be reported in this fashion. These features can also be reported as percentages of a library, as a score or percentile or normalized value calculated elsewhere, or as a value from a matrix or appropriately formatted dataset that provides this information for each cell or for each set of cells within a clonotype or an exact subclonotype.

In accordance with various embodiments, for each exact subclonotype, the textual frame can provide chain-specific subclonotype information selected from the group consisting of V(D)J UMI count, V(D)J read count, constant region name, complementarity-determining region (CDR) sequences for any of the CDR1/CDR2/CDR3 regions, constant sequence length, 5′UTR sequence length, differences from a universal reference constant region, differences from the 5′UTRsequence, base differences between exact subclonotypes, framework region amino acid and nucleotide sequences and lengths for any of FWR1/FWR2/FWR3/FWR4, and combinations thereof.

In accordance with various embodiments, the GUI can further include a user input section to receive information (e.g., via user input) configured to customize the display of immune cell clonotyping information relevant to one or more clonotypes, exact subclonotypes, or barcodes.

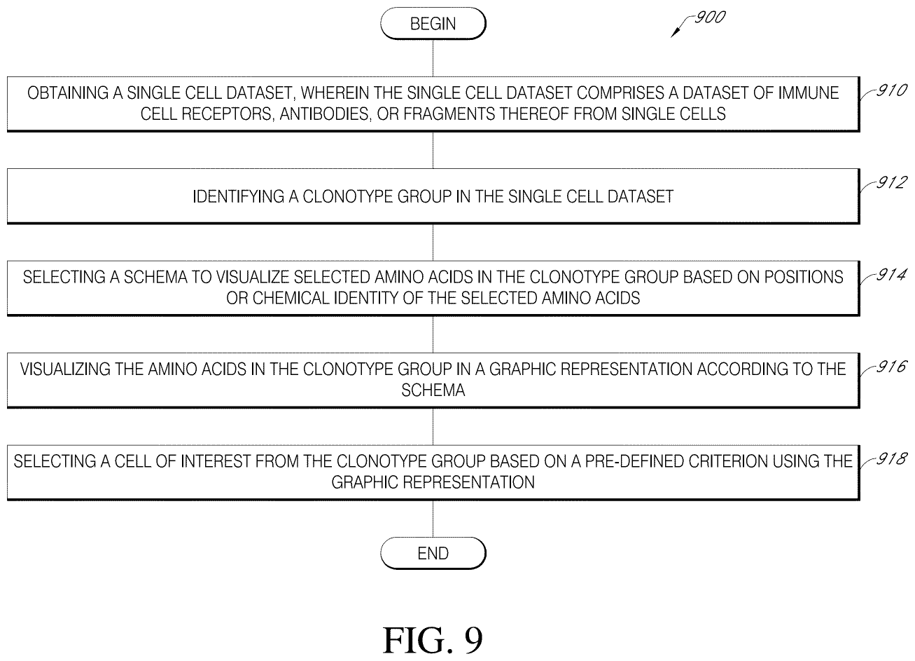

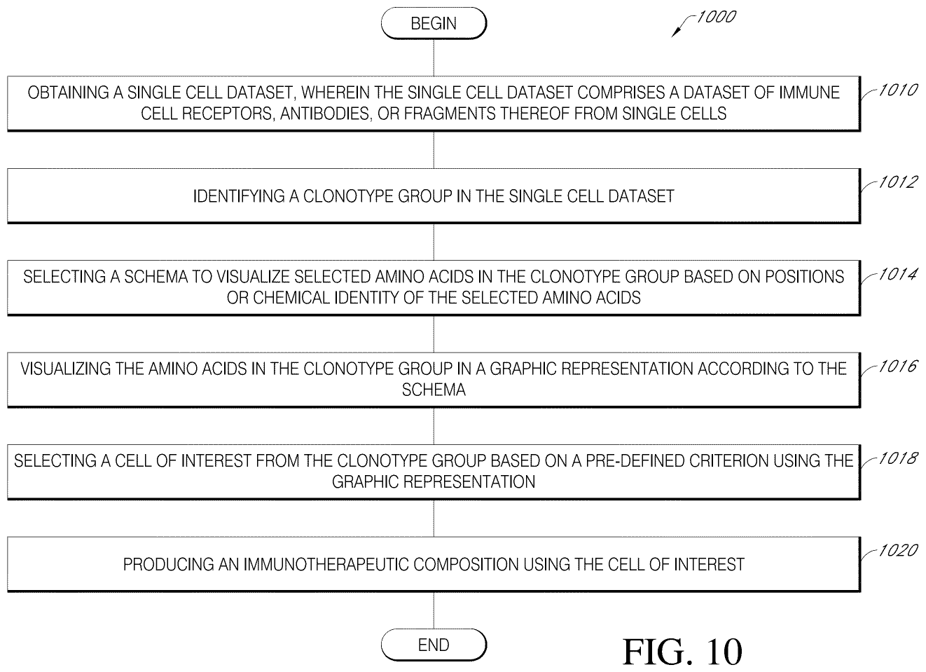

Methods (e.g., FIG. 9 or FIG. 10) are provided for selecting cells of interest or developing therapeutic compositions based on visualization of immune cell data (e.g., single cell dataset(s) or spatial dataset(s)). The exemplary methods in FIG. 9 and FIG. 10 are described with respect to a single cell dataset obtained from a sample of single cells. These methods may be similarly implemented for a spatial dataset that is obtained from a sample such as a tissue sample. For example, the same steps in FIGS. 9 and 10 may be implemented but with respect to a spatial dataset instead of a single cell dataset. The methods can be implemented via computer software or hardware. The methods can also be implemented on a computing device/system (e.g., FIG. 11 or FIG. 12) that can include a combination of engines or devices selecting cells of interest based on visualization of immune cell data. In one or more embodiments, at least a portion or all steps of the methods may be implemented using the clonotyping information visualizer, which may be implemented using, for example, without limitation, computer system 1100 in FIG. 11 or computer system 1200 in FIG. 12. In various embodiments, the computing device/system can be communicatively connected to one or more of a data source, sample analyzer (e.g., a genomic sequence analyzer), and display device via a direct connection or through an internet connection.

Referring now to FIG. 9, a flowchart illustrating a non-limiting example method 900 for selecting a cell of interest based on a single cell dataset is disclosed, in accordance with various embodiments. The method 900 can comprise, at step 910, obtaining a single cell dataset. The single cell dataset can include a dataset of at least one of immune cell receptors, antibodies, or fragments thereof from single cells. For example, the single cell dataset comprises a dataset of a B cell receptor, a T cell receptor, an antibody, a single-chain variable fragment (ScFv), an antigen-binding fragment (Fab), or a combination thereof.

The method 900 can comprise, at step 912, identifying a clonotype group in the single cell dataset. For example, there are several approaches for extracting CDR data from sequencing reads and determining the clonotype. One of the commonly used strategies for characterizing CDR3 sequences is antibody or TCR profiling, which amplifies cDNA or genomic DNA from the antibody or TCR (β-chain CDR3 (β-CDR3) locus using predesigned PCR primers, followed by deep sequencing. Another approach, antibody or TCR profiling based on RNA sequencing (RNA-seq), is more informative, providing data from all transcribed genes present in the sample as well as enabling simultaneous analysis of antibody and TCR chains.

The method 900 can comprise, at step 914, selecting a schema to visualize selected amino acids in the clonotype group based on at least one of positions or chemical identity of the selected amino acids. For example, the schema comprises selecting amino acids with a frequency meeting a preselected frequency threshold in the clonotype, selecting amino acids with a selected chemical identity in the clonotype, selecting amino acids of protein motifs that encode post-translational modification in the clonotype, displaying positions of selected amino acids in the clonotype group, displaying positions of selected amino acids in the clonotype group as in complementarity determining regions (CDR) or framework regions (FWR), highlighting selected amino acids in the clonotype group, or a combination thereof.

The method 900 can comprise, at step 916, visualizing the selected amino acids in the clonotype group in a graphic representation according to the schema. For example, the graphic representation comprises an alignment of one or more amino acid sequences of exact subclonotypes in the clonotype group, a comparison of at least one reference sequence to one or more amino acid sequences in the clonotype group, a phylogenetic tree of the clonotype group, or a combination thereof. The method 900 can further comprise building a phylogenetic tree of the clonotype group according to the schema and calculating a distance between each two exact subclonotypes in the clonotype group and between each two exact subclonotypes and a reference sequence.

The method 900 can comprise, at step 918, selecting a cell of interest from the clonotype group based on a pre-defined criterion using the graphic representation. For example, selecting the cell of interest from the clonotype group comprises selecting the cell of interest that have a constant region meeting a pre-defined constant region criterion and that have a distance between the cell of interest and a reference sequence at a heavy chain and a light chain level meeting a pre-defined distance criterion.

Referring now to FIG. 10, a flowchart illustrating a non-limiting example method 1000 for producing an immunotherapeutic composition from cells selected from a single cell dataset is disclosed, in accordance with various embodiments.

The method 1000 can comprise, at step 1010, obtaining a single cell dataset, wherein the single cell dataset comprises a dataset of at least one of immune cell receptors, antibodies, or fragments thereof from single cells. The method 1000 can comprise, at step 1012, identifying a clonotype group in the single cell dataset. The method 1000 can comprise, at step 1014, selecting a schema to visualize selected amino acids in the clonotype group based on positions or chemical identity of the selected amino acids. The method 1000 can comprise, at step 1016, visualizing the selected amino acids in the clonotype group in a graphic representation according to the schema. The method 1000 can comprise, at step 1018, selecting a cell of interest from the clonotype group based on a pre-defined criterion using the graphic representation. The method 1000 can comprise, at step 1020, producing an immunotherapeutic composition using the cell of interest.

While the exemplary methods and visualization schemas described above have been generally described with respect to single cell datasets, such methods or similar methods and visualization schemas may be used for spatial datasets, where a spatial dataset includes a dataset of at least one of immune cell receptors, antibodies, or fragments thereof from a tissue sample. For example, the spatial dataset may include a dataset of an antigen binding molecule (ABM), e.g., B cell receptor, a T cell receptor, an antibody, a single-chain variable fragment (ScFv), an antigen-binding fragment (Fab), or a combination thereof as obtained from a sample (e.g., a tissue sample).

### V. Methodologies for Treatment

The visualization methods and systems discussed herein may be used for treating or preventing disease or illness. The visualization methods and systems discussed herein may be used for treating or preventing disease or illness via the administration of one or more antibodies and/or one or more antigen-binding fragments to a subject, which have been isolated and selected based on the visualization schemas described herein.

For example, visualization methods and systems discussed herein may be used for treating or preventing a viral infection (e.g., reducing the likelihood of a viral infection such as coronavirus infection) by administering a composition comprising therapeutically effective amount of an anti-CoV-S antigen-binding polypeptide, e.g., antibody or antigen-binding fragment to a subject in need of such treatment or prevention. One or more embodiments may relate to methods for reducing binding of the SARS-Co-2V S protein to and/or reducing SARS-CoV-2 entry into a cell of a subject, the method including administering to the subject a composition comprising a therapeutically effective amount of at least one antibody or antigen-binding fragment isolated and/or selected based on the visualization(s) provided by the visualization methods and systems described herein. The composition may include, for example, at least one, e.g., at least two, at least three, at least four, at least five, at least six, at least seven antibodies or antigen-binding fragments. In some embodiments, the composition includes (a) a first antibody or antigen-binding fragment having a binding affinity to a receptor binding domain (RBD) and a second antibody or antigen-binding fragment having a binding affinity to a full-length SARS-CoV-2 S protein (e.g. to the Si subunit of the full-length SARS-CoV-2 S protein); (b) a first antibody or antigen-binding fragment having a binding affinity to a RBD and a second antibody or antigen-binding fragment having a binding affinity to a nucleoprotein N-terminal domain (NTD) of a SARS-CoV-2 S protein; or (c) a first antibody or antigen-binding fragment having a binding affinity to a NTD and a second antibody or antigen-binding fragment having a binding affinity to a full-length SARS-CoV-2 S protein (e.g. to the Si subunit of the full-length SARS-CoV-2 S protein).

One or more embodiments may include administering a treatment, which may include, for example, administering an anti-CoV-S antigen-binding polypeptide to a subject having one or more signs or symptoms of a disease or infection, e.g., viral infection, for which the antigen-binding polypeptide is effective when administered to the subject at an effective or therapeutically effective amount or dose. An effective or therapeutically effective dose of anti-CoV-S antigen-binding polypeptide, e.g., antibody or antigen-binding fragment, for treating or preventing a viral infection refers to the amount of the antibody or fragment sufficient to alleviate one or more signs and/or symptoms of the infection in the treated subject, whether by inducing the regression or elimination of such signs and/or symptoms or by inhibiting the progression of such signs and/or symptoms. Health conditions and symptoms associated with SARS-CoV-2 infection include respiratory tract infections, often in the lower respiratory tract. Accordingly, some embodiments of the disclosure relate to methods of for reducing one or more signs or symptoms associated with coronavirus infection, such as high fever, dry cough, shortness of breath, pneumonia, gastro-intestinal symptoms such as diarrhea, organ failure (kidney failure and renal dysfunction), septic shock, and death in severe cases. In some embodiments, a sign or symptom of a coronavirus infection in a subject is survival or proliferation of virus in the body of the subject, e.g., as determined by viral titer assay (e.g., coronavirus propagation in embryonated chicken eggs or coronavirus spike protein assay). Other signs and symptoms of viral infection include, but are not limited to, fever or feeling feverish/chills, cough, sore throat, runny or stuffy nose, sneezing, muscle or body aches, headaches, fatigue (tiredness), vomiting, diarrhea, respiratory tract infection, chest discomfort, shortness of breath, bronchitis, and pneumonia.

One or more embodiments may also encompass prophylactically administering an anti-CoV-S antigen-binding polypeptide, e.g., antibody or antigen-binding fragment thereof of the present disclosure, to a subject who is at risk of viral infection so as to prevent such infection (e.g., reducing the likelihood of a viral infection). Passive antibody-based immunoprophylaxis has proven an effective strategy for preventing subject from viral infection. The preventive methods of the disclosure involve administering a composition comprising an anti-CoV-S antigen-binding polypeptide, e.g., antibody or antigen-binding fragment of the present disclosure, to a subject to inhibit the manifestation of a disease or infection (e.g., viral infection) in the body of a subject, for which the antibody or antigen-binding fragment is effective when administered to the subject at an effective or therapeutically effective amount or dose.

Although the above methods of treatment have been described with respect to treating or preventing, for example, a coronavirus infection, the visualization methods and systems discussed herein may be similarly used for treating or preventing other types of infections or other diseases or illnesses.

### VI. Computer-Implemented System

In various embodiments, any methods described herein can be implemented via software, hardware, firmware, or a combination thereof. That is, as depicted in FIG. 11, the methods disclosed herein can be implemented on a computer system such as computer system 1100 (e.g., a computing device/analytics server). The computer system 1100 can include a computing dcvicc/analytics server 1112, which can be communicatively connected to a data source 1104 and a display system 1106 via a direct connection or through a network connection (e.g., LAN, WAN, Internet, etc.). It should be appreciated that the computer system 1100 (e.g., including a computing device/analytics server 1112) depicted in FIG. 11 can comprise additional engines or components as needed by the particular application or system architecture.

The data source 1104 can be configured to obtain a single cell dataset, wherein the single cell dataset comprises a dataset of at least one of immune cell receptors, antibodies, or fragments thereof from single cells. The processor 1113 can be configured to perform a method, wherein the method comprises identifying a clonotype group in the single cell dataset; selecting a schema to visualize selected amino acids in the clonotype group based on positions or chemical identity of the selected amino acids; and visualizing the selected amino acids in the clonotype group in a graphic representation according to the schema. The display system 1106 can be configured to render a visualization of the amino acids in the clonotype group in a graphic representation according to the schema.

In accordance with various embodiments, processor 1114 of computing device/analytics server 1112 can be communicatively connected to data source 1104, display 1106, user input device 1108, and/or graphic user interface 1110. In various embodiments, processor 1114 can include various engines configured to carry out the functionality of processor 1114. It should be appreciated that each component (e.g.. engine, module, unit etc.) depicted as part of system 1100 (and described herein) can be implemented as hardware, firmware, software, or any combination thereof.

In various embodiments, processor 1114 can be implemented as an integrated instrument system assembly with any of data storage 1104, display 1106, user input device 1108, and/or graphic user interface 1110. That is. any combination of processor 1114, data storage 1104, display 1106, user input device 1108, and/or graphic user interface 1110 can be boused in the same housing assembly and communicate via conventional device/component connection means (e.g. serial bus, optical cabling, electrical cabling, etc.).

In various embodiments, processor 1114 can be implemented as a standalone computing device (as shown in FIG. 11) that can be communicatively connected to the data source 1004 (and likewise display 1106, user input device 1108, and graphic user interface 1110) via an optical, serial port, network or modem connection. For example, the processor 1114 can be connected via a LAN or WAN connection that allows for the transmission of data to and from the data source 1104, and likewise display 1106 and user input device 1108.

In various embodiments, the functions of processor 1114 can be implemented on a distributed network of shared computer processing resources (such as a cloud computing network) that is communicatively connected to the data storage 1104 via a WAN (or equivalent) connection. For example, the functionalities of processor 1114 can be divided up to be implemented in one or more computing nodes on a cloud processing service such as AMAZON WEB SERVICES™.

Within the processor 1114, any internal engines can be implemented as separate engines or a single multi-functional engine. As such, FIG. 11 simply provides one example implementation of a system in accordance with various embodiments, and should be not be read to limit the interchangeability, interoperability and/or functionality of all the components therein.

FIG. 12 is a block diagram that illustrates a computer system 1200, upon which embodiments of the present disclosure may be implemented. In various embodiments of the present teachings, computer system 1200 can include a bus 1202 or other communication mechanism for communicating information, and a processor 1204 coupled with bus 1202 for processing information. In various embodiments, computer system 1200 can also include a memory, which can be a random access memory (RAM) 1206 or other dynamic storage device, coupled to bus 1202 for determining instructions to be executed by processor 1204. Memory also can be used for storing temporary variables or other intermediate information during execution of instructions to be executed by processor 1204. In various embodiments, computer system 1200 can further include a read only memory (ROM) 1208 or other static storage device coupled to bus 1202 for storing static information and instructions for processor 1204. A storage device 1210, such as a magnetic disk or optical disk, can be provided and coupled to bus 1202 for storing information and instructions.

In various embodiments, computer system 1200 can be coupled via bus 1202 to a display 1212, such as a cathode ray tube (CRT) or liquid crystal display (LCD), for displaying information to a computer user. An input device 1214, including alphanumeric and other keys, can be coupled to bus 1202 for communicating information and command selections to processor 404. Another type of user input device is a cursor control 1216, such as a mouse, a trackball or cursor direction keys for communicating direction information and command selections to processor 1204 and for controlling cursor movement on display 1212. This input device 1214 typically has two degrees of freedom in two axes, a first axis (i.e., x) and a second axis (i.e., y), that allows the device to specify positions in a plane. However, it should be understood that input devices 1214 allowing for three dimensional (x, y and z) cursor movement are also contemplated herein.

Consistent with certain implementations of the present teachings, results can be provided by computer system 1200 in response to processor 1204 executing one or more sequences of one or more instructions contained in memory 1206. Such instructions can be read into memory 406 from another computer-readable medium or computer-readable storage medium, such as storage device 1210. Execution of the sequences of instructions contained in memory 1206 can cause processor 1204 to perform the processes described herein. Alternatively, hard-wired circuitry can be used in place of or in combination with software instructions to implement the present teachings. Thus, implementations of the present teachings are not limited to any specific combination of hardware circuitry and software.

The term “computer-readable medium” (e.g., data store, data storage, etc.) or “computer-readable storage medium” as used herein refers to any media that participates in providing instructions to processor 1204 for execution. Such a medium can take many forms, including but not limited to, non-volatile media, volatile media, and transmission media. Examples of non-volatile media can include, but are not limited to, optical, solid state, magnetic disks, such as storage device 1210. Examples of volatile media can include, but are not limited to, dynamic memory, such as memory 406. Examples of transmission media can include, but are not limited to, coaxial cables, copper wire, and fiber optics, including the wires that comprise bus 1202.

Common forms of computer-readable media include, for example, a floppy disk, a flexible disk, hard disk, magnetic tape, or any other magnetic medium, a CD-ROM, any other optical medium, punch cards, paper tape, any other physical medium with patterns of holes, a RAM, PROM, and EPROM, a FLASH-EPROM, any other memory chip or cartridge, or any other tangible medium from which a computer can read.

In addition to computer readable medium, instructions or data can be provided as signals on transmission media included in a communications apparatus or system to provide sequences of one or more instructions to processor 1204 of computer system 1200 for execution. For example, a communication apparatus may include a transceiver having signals indicative of instructions and data. The instructions and data are configured to cause one or more processors to implement the functions outlined in the disclosure herein. Representative examples of data communications transmission connections can include, but are not limited to. telephone modem connections, wide area networks (WAN), local area networks (LAN), infrared data connections, NFC connections, etc.

It should be appreciated that the methodologies described herein flow charts, diagrams and accompanying disclosure can be implemented using computer system 1200 as a standalone device or on a distributed network of shared computer processing resources such as a cloud computing network.

The methodologies described herein may be implemented by various means depending upon the application. For example, these methodologies may be implemented in hardware, firmware, software, or any combination thereof. For a hardware implementation, the processing unit may be implemented within one or more application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, electronic devices, other electronic units designed to perform the functions described herein, or a combination thereof.

In various embodiments, the methods of the present teachings may be implemented as firmware and/or a software program and applications written in conventional programming languages such as C, C++, Rust, Python, etc. If implemented as firmware and/or software, the embodiments described herein can be implemented on a non-transitory computer-readable medium in which a program is stored for causing a computer to perform the methods described above. It should be understood that the various engines described herein can be provided on a computer system, such as computer system 1200, whereby processor 1204 would execute the analyses and determinations provided by these engines, subject to instructions provided by any one of, or a combination of, memory components 1206/1208/1210 and user input provided via input device 1214.

### Digital Processing Device

In various embodiments, the systems and methods described herein can include a digital processing device, or use of the same. In various embodiments, the digital processing device can include one or more hardware central processing units (CPUs) or general-purpose graphics processing units (GPGPUs) that carry out the device's functions. In various embodiments, the digital processing device further comprises an operating system configured to perform executable instructions. In various embodiments, the digital processing device can be optionally connected a computer network. In various embodiments, the digital processing device can be optionally connected to the Internet such that it accesses the World Wide Web. In various embodiments, the digital processing device can be optionally connected to a cloud computing infrastructure. In various embodiments, the digital processing device can be optionally connected to an intranet. In various embodiments, the digital processing device can be optionally connected to a data storage device.

In accordance with various embodiments, suitable digital processing devices can include, by way of non-limiting examples, server computers, desktop computers, laptop computers, notebook computers, sub-notebook computers, netbook computers, netpad computers, handheld computers, Internet appliances, mobile smartphones, tablet computers, and personal digital assistants. Those of ordinary skill in the art will recognize that many smartphones are suitable for use in the system described herein. Those of ordinary skill in the art will also recognize that select televisions, video players, and digital music players with optional computer network connectivity are suitable for use in the system described herein. Suitable tablet computers include those with booklet, slate, and convertible configurations, known to those of ordinary skill in the art.

In various embodiments, the digital processing device includes an operating system configured to perform executable instructions. The operating system can be, for example, software, including programs and data, which manages the device's hardware and provides services for execution of applications. Those of ordinary skill in the art will recognize that suitable server operating systems include, by way of non-limiting examples, FreeB SD, OpenBSD, Net-BSD, Linux, Apple® Mac OS X Server®, Oracle® Solaris®, Windows Server®, and Novell® NetWare®. Those of ordinary skill in the art will recognize that suitable personal computer operating systems include, by way of non-limiting examples, Microsoft® Windows®, Apple® Mac OS X®, UNIX®, and UNIX-like operating systems such as GNU/Linux®. In various embodiments, the operating system is provided by cloud computing. Those of ordinary skill in the art will also recognize that suitable mobile smart phone operating systems include, by way of non-limiting examples, Nokia® Symbian® OS, Apple® iOS®, Research In Motion® Black- Berry OS®, Google® Android®, Microsoft® Windows Phone® OS, Microsoft® Windows Mobile® OS, Linux®, and Palm® WebOS®.

In various embodiments, the device includes a storage and/or memory device. The storage and/or memory device is one or more physical apparatuses used to store data or programs on a temporary or permanent basis. In various embodiments, the device is volatile memory and requires power to maintain stored information. In various embodiments, the device is non-volatile memory and retains stored information when the digital processing device is not powered. In various embodiments, the non-volatile memory comprises flash memory. Tn some embodiments, the non-volatile memory comprises dynamic random-access memory (DRAM). In various embodiments, the non-volatile memory comprises ferroelectric random access memory (FRAM). In various embodiments, the nonvolatile memory comprises phase-change random access memory (PRAM). In various embodiments, the device is a storage device including, by way of non-limiting examples, CD-ROMs, DVDs, flash memory devices, magnetic disk drives, magnetic tapes drives, optical disk drives, and cloud computing-based storage. In various embodiments, the storage and/or memory device is a combination of devices such as those disclosed herein.

In various embodiments, the digital processing device includes a display to send visual information to a user. In various embodiments, the display is a cathode ray tube (CRT). In various embodiments, the display is a liquid crystal display (LCD). In various embodiments, the display is a thin film transistor liquid crystal display (TFT-LCD). In various embodiments, the display is an organic light emitting diode (OLED) display. In various embodiments, on OLED display is a passive-matrix OLED (PMOLED) or active- matrix OLED (AMOLED) display. In various embodiments, the display is a plasma display. In various embodiments, the display is a video projector. In various embodiments, the display is a combination of devices such as those disclosed herein.

In various embodiments, the digital processing device includes an input device to receive information from a user. In various embodiments, the input device is a keyboard. In various embodiments, the input device is a pointing device including, by way of non-limiting examples, a mouse, trackball, track pad, joystick, game controller, or stylus. In various embodiments, the input device is a touch screen or a multi-touch screen. In various embodiments, the input device is a microphone to capture voice or other sound input. In various embodiments, the input device is a video camera or other sensor to capture motion or visual input. In various embodiments, the input device is a Kinect, Leap Motion, or the like. In various embodiments, the input device is a combination of devices such as those disclosed herein.

### Non-Transitory Computer Readable Storage Medium

In various embodiments, and as stated above, the systems and methods disclosed herein can include, and the methods herein can be run on, one or more non-transitory computer readable storage media encoded with a program including instructions executable by the operating system of an optionally networked digital processing device. In various embodiments, a computer readable storage medium is a tangible component of a digital processing device. In various embodiments, a computer readable storage medium is optionally removable from a digital processing device. In various embodiments, a computer readable storage medium includes, by way of non-limiting examples, CD-ROMs, DVDs, flash memory devices, solid state memory, magnetic disk drives, magnetic tape drives, optical disk drives, cloud computing systems and services, and the like. In various embodiments, the program and instructions are permanently, substantially permanently, semi-permanently, or non-transitorily encoded on the media.

### Computer Program

In various embodiments, the systems and methods disclosed herein can include at least one computer program, or use at least one computer program. A computer program includes a sequence of instructions, executable in the digital processing device's CPU, written to perform a specified task. Computer readable instructions may be implemented as program modules, such as functions, objects, Application Programming Interfaces (APis), data structures, and the like, that perform particular tasks or implement particular abstract data types. Those of ordinary skill in the art will recognize that a computer program may be written in various versions of various languages.

The functionality of the computer readable instructions may be combined or distributed as desired in various environments. In various embodiments, a computer program comprises one sequence of instructions. In various embodiments, a computer program comprises a plurality of sequences of instructions. In various embodiments, a computer program is provided from one location. In various embodiments, a computer program is provided from a plurality of locations. In various embodiments, a computer program includes one or more software modules. In various embodiments, a computer program includes, in part or in whole, one or more web applications, one or more mobile applications, one or more standalone applications, one or more web browser plug-ins, extensions, add-ins, or add-ons, or combinations thereof.

### Web Application

In various embodiments, a computer program includes a web application. Those of ordinary skill in the art will recognize that a web application, in various embodiments, utilizes one or more software frameworks and one or more database systems. In various embodiments, a web application is created upon a software framework such as Microsoft® .NET or Ruby on Rails (RoR). In various embodiments, a web application utilizes one or more database systems including, by way of non-limiting examples, relational, non-relational, object oriented, associative, and XML database systems. In various embodiments, suitable relational database systems include, by way of non-limiting examples, Microsoft® SQL Server, mySQL™, and Oracle®. Those of ordinary skill in the art will also recognize that a web application, in various embodiments, is written in one or more versions of one or more languages. A web application may be written in one or more markup languages, presentation definition languages, client- side scripting languages, server-side coding languages, data- base query languages, or combinations thereof. In various embodiments, a web application is written to some extent in a markup language such as Hypertext Markup Language (HTML), Extensible Hypertext Markup Language (XHTML), or extensible Markup Language (XML). In various embodiments, a web application is written to some extent in a presentation definition language such as Cascading Style Sheets (CS S). In various embodiments, a web application is written to some extent in a client-side scripting language such as Asynchronous Javascript and XML (AJAX), Flash® Actionscript, Javascript, or Silverlight®. In various embodiments, a web application is written to some extent in a server-side coding language such as Active Server Pages (ASP), ColdFusion®, Perl, Java™, JavaServer Pages (JSP), Hypertext Preprocessor (PHP), Python™, Ruby, Tel, Smalltalk, WebDNA®, or Groovy. In various embodiments, a web application is written to some extent in a database query language such as Structured Query Language (SQL). In various embodiments, a web application integrates enterprise server products such as IBM® Lotus Domino®. In various embodiments, a web application includes a media player element. In various embodiments, a media player element utilizes one or more of many suitable multimedia technologies including, by way of non-limiting examples, Adobe® Flash®, HTML 5, Apple® QuickTime®, Microsoft® Silverlight®, Java™, and Unity®.

### Mobile Application

In various embodiments, a computer program includes a mobile application provided to a mobile digital processing device. In various embodiments, the mobile application is provided to a mobile digital processing device at the time it is manufactured. In various embodiments, the mobile application is provided to a mobile digital processing device via the computer network described herein.

A mobile application can be created by techniques known to those of ordinary skill in the art using hardware, languages, and development environments known to the art. Those of ordinary skill in the art will recognize that mobile applications can be written in several languages. Suitable programming languages include, by way of non-limiting examples, C, C++, C#, Objective-C, Java™, JavaScript, Pascal, Object Pascal, Rust, Python™, Ruby, VB.NET, WML, and XHTML/HTML with or without CSS, or combinations thereof.

Suitable mobile application development environments are available from several sources. Commercially available development environments include, by way of non-limiting examples, AirplaySDK, alcheMo, Appcelera-tor®, Celsius, Bedrock, Flash Lite, .NET Compact Frame-work, Rhomobile, and WorkLight Mobile Platform. Other development environments are available without cost including, by way of non-limiting examples, Lazarus, Mobi-Flex, MoSync, and Phonegap. Also, mobile device manufacturers distribute software developer kits including, by way of non-limiting examples, iPhone and iPad (iOS) SDK, Android™ SDK, BlackBerry® SDK. BREW SDK, Palm® OS SDK, Symbian SDK, webOS SDK, and Windows® Mobile SDK.

Those of ordinary skill in the art will recognize that several commercial forums are available for distribution of mobile applications including, by way of non-limiting examples, Apple® App Store, Google® Play, Chrome WebStore, BlackBerry® App World, App Store for Palm devices, App Catalog for webOS, Windows® Marketplace for Mobile, Ovi Store for Nokia® devices, Samsung® Apps, and Nin-tendo DSi Shop.

sStandalone Application

In various embodiments, a computer program includes a standalone application, which is a program that is run as an independent computer process, not an add-on to an existing process, e.g., not a plug-in. Those of ordinary skill in the art will recognize that standalone applications are often compiled. A compiler is a computer program(s) that transforms source code written in a programming language into binary object code such as assembly language or machine code. Suitable compiled programming languages include, by way of non-limiting examples, Rust, C, C++, Objective-C, COBOL, Delphi, Eiffel, Java™, Lisp, Python™, Visual Basic, and VB.NET, or combinations thereof. Compilation is often per- formed, at least in part, to create an executable program. In various embodiments, a computer program includes one or more executable complied applications.

### Web Browser Plug-in

In various embodiments, the computer program includes a web browser plug-in (e.g., extension, etc.). In computing, a plug-in is one or more software components that add specific functionality to a larger software application. Makers of software applications support plug-ins to enable third-party developers to create abilities, which extend an application, to support easily adding new features, and to reduce the size of an application. When supported, plug-ins enable customizing the functionality of a software application. For example, plug-ins are commonly used in web browsers to play video, generate interactivity, scan for viruses, and display particular file types. Those of ordinary skill in the art will be familiar with several web browser plug-ins including, Adobe® Flash® Player, Microsoft® Silver- light®, and Apple® QuickTime®. In various embodiments, the toolbar comprises one or more web browser extensions, add-ins, or add-ons. In various embodiments, the toolbar comprises one or more explorer bars, tool bands, or desk bands.

Those of ordinary skill in the art will recognize that several plug-in frame works are available that enable development of plug-ins in various programming languages, including, by way of non-limiting examples, C++, Delphi, Java™, PHP, Python™, and VB .NET, or combinations thereof.

Web browsers (also called Internet browsers) are software applications, designed for use with network-connected digital processing devices, for retrieving, presenting, and traversing information resources on the World Wide Web. Suitable web browsers include, by way of non-limiting examples, Microsoft® Internet Explorer®, Mozilla® Fire- fox®, Google® Chrome, Apple® Safari®, Opera Soft- ware® Opera®, and KDE Konqueror. In various embodiments, the web browser is a mobile web browser. Mobile web browsers (also called mircrobrowsers, mini-browsers, and wireless browsers) are designed for use on mobile digital processing devices including, by way of non-limiting examples, handheld computers, tablet computers, netbook computers, subnotebook computers, smartphones, and personal digital assistants (PDAs). Suitable mobile web browsers include, by way of non-limiting examples, Google® Android® browser, RIM BlackBerry® Browser, Apple® Safari®, Palm® Blazer, Palm® WebOS® Browser, Mozilla® Firefox® for mobile, Microsoft® Internet Explorer® Mobile. Amazon® Kindle® Basic Web. Nokia® Browser, Opera Software® Opera® Mobile, and Sony PSP™ browser.

### Software Modules

In various embodiments, the systems and methods disclosed herein include a software, server and/or database modules, or incorporate use of the same in methods according to various embodiments disclosed herein. Software modules can be created by techniques known to those of ordinary skill in the art using machines, software, and languages known to the art. The software modules disclosed herein are implemented in a multitude of ways. In various embodiments, a software module comprises a file, a section of code, a programming object, a programming structure, or combinations thereof. In further various embodiments, a software module comprises a plurality of files, a plurality of sections of code, a plurality of programming objects, a plurality of programming structures, or combinations thereof. In various embodiments, the one or more software modules comprise, by way of non-limiting examples, a web application, a mobile application, and a standalone application. In various embodiments, software modules are in one computer program or application. In various embodiments, software modules are in more than one computer program or application. In various embodiments, software modules are hosted on one machine. In various embodiments, software modules are hosted on more than one machine. In various embodiments, software modules are hosted on cloud computing platforms. In various embodiments, software modules are hosted on one or more machines in one location. In various embodiments, software modules are hosted on one or more machines in more than one location.

### Databases

In various embodiments, the systems and methods disclosed herein include one or more databases, or incorporate use of the same in methods according to various embodiments disclosed herein. Those of ordinary skill in the art will recognize that many databases are suitable for storage and retrieval of user, query, token, and result information. In various embodiments, suitable databases include, by way of non-limiting examples, relational databases, non-relational databases, object oriented databases, object databases, entity-relation- ship model databases, associative databases, and XML databases. Further non-limiting examples include SQL, Postgr-eSQL, MySQL, Oracle, DB2, and Sybase. In various embodiments, a database is internet-based. In further Web. Suitable web browsers include, by way of non-limiting examples, Microsoft®

Internet Explorer®, Mozilla® Fire- fox®, Google® Chrome, Apple® Safari®, Opera Soft- ware® Opera®, and KDE Konqueror. In various embodiments, the web browser is a mobile web browser. Mobile web browsers (also called microbrowsers, mini-browsers, and wireless browsers) are designed for use on mobile digital processing devices including, by way of non-limiting examples, handheld computers, tablet computers, netbook computers, subnotebook computers, smartphones, and personal digital assistants (PDAs). Suitable mobile web browsers include, by way of non-limiting examples, Google® Android® browser, RIM BlackBerry® Browser, Apple® Safari®, Palm® Blazer, Palm® WebOS® Browser, Mozilla® Firefox® for mobile, Microsoft® Internet Explorer® Mobile, Amazon® Kindle® Basic Web, Nokia® Browser, Opera Software® Opera® Mobile, and Sony P SP™ browser.

In various embodiments, a database is web-based. In various embodiments, a database is cloud computing-based. In other embodiments, a database is based on one or more local computer storage devices.

### Data Security

In various embodiments, the systems and methods disclosed herein include one or features to prevent unauthorized access. The security measures can, for example, secure a user's data. In various embodiments, data is encrypted. In various embodiments, access to the system requires multi-factor authentication and access control layer. In various embodiments, access to the system requires two-step authentication (e.g., web-based interface). In various embodiments, two-step authentication requires a user to input an access code sent to a user's e-mail or cell phone in addition to a username and password. In some instances, a user is locked out of an account after failing to input a proper username and password. The systems and methods disclosed herein can, in various embodiments, also include a mechanism for protecting the anonymity of users' genomes and of their searches across any genomes.

### VII. Recitation of Embodiments

Embodiment 1. A method for selecting a cell of interest based on a single cell dataset, the method comprising: obtaining a single cell dataset, wherein the single cell dataset comprises a dataset of immune cell receptors, antibodies, or fragments thereof from single cells;

identifying a clonotype group in the single cell dataset; selecting a schema to visualize selected amino acids in the clonotype group based on positions or chemical identity of the selected amino acids; visualizing the selected amino acids in the clonotype group in a graphic representation according to the schema; and selecting a cell of interest from the clonotype group based on a pre-defined criterion using the graphic representation.

Embodiment 2. The method of embodiment 1, wherein the single cell dataset comprises a dataset of a B cell receptor, a T cell receptor, an antibody, a single-chain variable fragment (ScFv), an antigen-binding fragment (Fab), or a combination thereof

Embodiment 3. The method of embodiment 1 or embodiment 2, wherein the schema comprises selecting amino acids with a frequency meeting a pre-selected frequency threshold in the clonotype group.

Embodiment 4. The method of any one of embodiments 1-3, wherein the schema comprises selecting amino acids with a selected chemical identity in the clonotype group.

Embodiments 5. The method of any one of embodiments 1-4, wherein the schema comprises selecting amino acids of protein motifs that encode post-translational modification in the clonotype group.

Embodiment 6. The method of any one of embodiments 1-5, wherein the schema comprises displaying positions of the selected amino acids in the clonotype group.

Embodiment 7. The method of any one of embodiments 1-6, wherein the schema comprises displaying positions of the selected amino acids in the clonotype group as in complementarity determining regions (CDR) or framework regions (FWR).

Embodiment 8. The method of any one of embodiments 1-7, wherein the schema comprises highlighting selected amino acids in the clonotype group.

Embodiment 9. The method of any one of embodiments 1-8, wherein the graphic representation comprises an alignment of one or more amino acid sequences of exact subclonotypes in the clonotype group.

Embodiment 10. The method of any one of embodiments 1-9, wherein the graphic representation comprises a comparison of at least one reference sequence to one or more amino acid sequences in the clonotype group.

Embodiment 11. The method of any one of embodiments 1-10, wherein the graphic representation comprises a phylogenetic tree of the clonotype group.

Embodiment 12. The method of any one of embodiments 1-11, further comprising: building a phylogenetic tree of the clonotype group according to the schema; and calculating a distance between each two exact subclonotypes in the clonotype group and between each exact subclonotype and a reference sequence.

Embodiment 13. The method of any one of embodiments 1-12, wherein selecting the cell of interest from the clonotype group comprises: selecting the cell of interest that has a constant region meeting a pre-defined constant region criterion and that has a distance between the cell of interest and a reference sequence at a heavy chain and a light chain level meeting a pre-defined distance criterion.

Embodiment 14. An interactive visualization system comprising:
a data source for obtaining a single cell dataset, wherein the single cell dataset comprises a dataset of immune cell receptors, antibodies, or fragments thereof from single cells;
one or more data processors;
a computing device communicatively connected to the data source and configured to receive the single cell dataset, the computing device comprising a non-transitory computer readable storage medium containing instructions which, when executed on the one or more data processors, cause the one or more data processors to perform a method, the method comprising:
   identifying a clonotype group in the single cell dataset;
   selecting a schema to visualize selected amino acids in the clonotype group based on positions or chemical identity of the selected amino acids; and
   visualizing the selected amino acids in the clonotype group in a graphic representation according to the schema; and
   a display for rendering a visualization of the selected amino acids in the clonotype group in the graphic representation according to the schema.

Embodiment 15. The system of embodiment 14, further comprising: a user input device for receiving a user-selected parameter under which to analyze the dataset.

Embodiment 16. The system of any one of embodiment 14 or embodiment 15, wherein the single cell dataset comprises a dataset of a B cell receptor, a T cell receptor, an antibody, a single-chain variable fragment (ScFv), an antigen-binding fragment (Fab), or a combination thereof

Embodiment 17. The system of any one of embodiments 14-16, wherein the schema comprises selecting amino acids with a frequency meeting a pre-selected frequency threshold in the clonotype group.

Embodiment 18. The system of any one of embodiments 14-17, wherein the schema comprises selecting amino acids with a selected chemical identity in the clonotype group.

Embodiment 19. The system of any one of embodiments 14-18, wherein the schema comprises selecting amino acids of protein motifs that encode post-translational modification in the clonotype group.

Embodiment 20. The system of any one of embodiments 14-19, wherein the schema comprises displaying positions of the selected amino acids in the clonotype group.

Embodiment 21. The system of any one of embodiments 14-20, wherein the schema comprises displaying positions of the selected amino acids in the clonotype group as in complementarity determining regions (CDR) or framework regions (FWR).

Embodiment 22. The system of any one of embodiments 14-21, wherein the schema comprises highlighting the selected amino acids in the clonotype group.

Embodiment 23. The system of any one of embodiments 14-22, wherein the graphic representation comprises an alignment of one or more amino acid sequences in the clonotype group.

Embodiment 24. The system of any one of embodiments 14-23, wherein the graphic representation comprises a comparison of at least one reference sequence to one or more sequences of peptides in the clonotype group.

Embodiment 25. The system of any one of embodiments 14-24, wherein the graphic representation comprises a phylogenetic tree of the clonotype group.

Embodiment 26. The system of any one of embodiments 14-25, wherein the method further comprises building a phylogenetic tree of the clonotype group according to the schema.

Embodiment 27. A method for producing an immunotherapeutic composition from cells selected from a single cell dataset, the method comprising: obtaining a single cell dataset, wherein the single cell dataset comprises a dataset of immune cell receptors, antibodies, or fragments thereof from single cells; identifying a clonotype group in the single cell dataset; selecting a schema to visualize selected amino acids in the clonotype group based on positions or chemical identity of the selected amino acids; visualizing the selected amino acids in the clonotype group in a graphic representation according to the schema; selecting a cell of interest from the clonotype group based on a pre-defined criterion using the graphic representation; and producing an immunotherapeutic composition using the cell of interest.

Embodiment 28. A graphical user interface (GUI) for displaying immune cell clonotyping information, the GUI comprising: a listing of exact subclonotypes of an immune cell clonotype, wherein the exact subclonotypes share identical V(D)J transcripts; a listing of one or more textual frames with information about chains common to each member of the immune cell clonotype, wherein a textual frame of the one or more textual frames contains an amino acid sequence for variable and constant regions of each exact subclonotype of the exact subclonotypes; and positional information for selected amino acids of the amino acid sequence, wherein the selected amino acids are selected based on positions or chemical identity of the selected amino acids.

Embodiment 29. The graphical user interface of embodiment 28, wherein the listing of the one or more textual frames comprises two or more textual frames.

Embodiment 30. The graphical user interface of embodiment 28 or embodiment 29, wherein the listing of the one or more textual frames comprises two textual frames.

Embodiment 31. The graphical user interface of any one of embodiments 28-30, wherein the listing of the one or more textual frames comprises three textual frames.

Embodiment 32. The graphical user interface of any one of embodiments 28-31, wherein the listing of the one or more textual frames includes a comparison of at least one reference sequence to an exact subclonotype of the exact subclonotypes.

Embodiment 3 3. The graphical user interface of embodiment 3 2, wherein the at least one reference sequence includes a reference sequence listing selected from the group consisting of a universal reference sequence, a donor reference sequence, and combinations thereof.

Embodiment 34. The graphical user interface of any one of embodiments 28-33, wherein the listing of the one or more textual frames includes a listing of amino acid alignments of each exact subclonotypes of the immune cell clonotype.

Embodiment 35. The graphical user interface of any one of embodiments 28-34, wherein the listing of the exact subclonotypes includes subclonotype information selected from the group consisting of gene expression, Hamming distance, antibody, and combinations thereof.

Embodiment 36. The graphical user interface of embodiment 35, wherein the gene expression of the subclonotype information is selected from the group consisting of median gene expression, maximum gene expression, mean gene expression, and combinations thereof

Embodiment 37. The graphical user interface of embodiment 35 or embodiment 36, wherein the gene expression of the subclonotype information is reported as a UMI count.

Embodiment 38. The graphical user interface of any one of embodiments 28-37, wherein for each exact subclonotype of the exact subclonotypes, the textual frame provides chain-specific subclonotype information selected from the group consisting of V(D)J UMI count, V(D)J read count, constant region name, complementarity-determining region (CDR) sequence, constant sequence length, 5′UTR sequence length, differences from a universal reference constant region, differences from the 5′UTR sequence, base differences between exact subclonotypes, and combinations thereof.

Embodiment 39. The graphical user interface of any one of embodiments 28-38, further comprising a user input section to receive information configured to customize a display of immune cell clonotyping information.

Embodiment 40. A computer-program product tangibly embodied in a non-transitory machine-readable storage medium, including instructions configured to cause one or more data processors to perform a method for selecting a cell of interest based on a single cell dataset, the method comprising: obtaining a single cell dataset, wherein the single cell dataset comprises a dataset of immune cell receptors, antibodies, or fragments thereof from single cells; identifying a clonotype group in the single cell dataset; selecting a schema to visualize selected amino acids in the clonotype group based on positions or chemical identity of the selected amino acids; visualizing the selected amino acids in the clonotype group in a graphic representation according to the schema; and selecting a cell of interest from the clonotype group based on a pre-defined criterion using the graphic representation.

Embodiment 41. A method for selecting a cell of interest based on immune cell data, the method comprising: obtaining the immune cell data, wherein the immune cell data comprises a dataset of immune cell receptors, antibodies, or fragments thereof from a sample; identifying a clonotype group in the immune cell data; selecting a schema to visualize selected amino acids in the clonotype group based on positions or chemical identity of the selected amino acids; visualizing the selected amino acids in the clonotype group in a graphic representation according to the schema; and selecting a cell of interest from the clonotype group based on a pre-defined criterion using the graphic representation.

Embodiment 42. The method of embodiment 41, wherein the immune cell data comprises a dataset of a B cell receptor, a T cell receptor, an antibody, a single-chain variable fragment (ScFv), an antigen-binding fragment (Fab), or a combination thereof

Embodiment 43. The method of embodiment 41 or embodiment 42, wherein the schema comprises selecting amino acids with a frequency meeting a pre-selected frequency threshold in the clonotype group.

Embodiment 44. The method of any one of embodiments 41-43, wherein the schema comprises selecting amino acids with a selected chemical identity in the clonotype group.

Embodiment 45. The method of any one of embodiments 41-44, wherein the schema comprises selecting amino acids of protein motifs that encode post-translational modification in the clonotype group.

Embodiment 46. The method of any one of embodiments 41-45, wherein the schema comprises displaying positions of the selected amino acids in the clonotype group.

Embodiment 47. The method of any one of embodiments 41-46, wherein the schema comprises displaying positions of the selected amino acids in the clonotype group as in complementarity determining regions (CDR) or framework regions (FWR).

Embodiment 48. The method of any one of embodiments 41-47, wherein the schema comprises highlighting selected amino acids in the clonotype group.

Embodiment 49. The method of any one of embodiments 41-48, wherein the graphic representation comprises an alignment of one or more amino acid sequences of exact subclonotypes in the clonotype group.

Embodiment 50. The method of any one of embodiments 41-49, wherein the graphic representation comprises a comparison of at least one reference sequence to one or more amino acid sequences in the clonotype group.

Embodiment 51. The method of any one of embodiments 41-50, wherein the graphic representation comprises a phylogenetic tree of the clonotype group.

Embodiment 52. The method of any one of embodiments 41-51, further comprising: building a phylogenetic tree of the clonotype group according to the schema; and calculating a distance between each two exact subclonotypes in the clonotype group and between each exact subclonotype and a reference sequence.

Embodiment 53. The method of any one of embodiments 41-52, wherein selecting the cell of interest from the clonotype group comprises: selecting the cell of interest that has a constant region meeting a pre-defined constant region criterion and that has a distance between the cell of interest and a reference sequence at a heavy chain and a light chain level meeting a pre-defined distance criterion.

Embodiment 54. The method of any one of embodiments 41-53, wherein the sample comprises single cells and the immune cell data comprises a single cell dataset obtained from the single cells.

Embodiment 55. The method of any one of embodiments 41-53, wherein the sample comprises a tissue sample and the immune cell data comprises a spatial dataset obtained from the tissue sample.

Embodiment 56. An interactive visualization system comprising:
a data source for obtaining immune cell data, wherein the immune cell data comprises a dataset of immune cell receptors, antibodies, or fragments thereof from a sample;
a computing device communicatively connected to the data source and configured to receive the immune cell data, the computing device comprising a set of processors and a non-transitory computer readable storage medium containing instructions which, when executed by the set of processors, cause the set of processors to perform a method comprising:
   identifying a clonotype group in the immune cell data;
   selecting a schema to visualize selected amino acids in the clonotype group based on positions or chemical identity of the selected amino acids; and
   visualizing the selected amino acids in the clonotype group in a graphic representation according to the schema; and
   a display for rendering a visualization of the selected amino acids in the clonotype group in the graphic representation according to the schema.

Embodiment 57. The interactive visualization system of embodiment 56, wherein the sample comprises single cells and the immune cell data comprises a single cell dataset obtained from the single cells.

Embodiment 58. The interactive visualization system of embodiment 56, wherein the sample comprises a tissue sample and the immune cell data comprises a spatial dataset obtained from the tissue sample.

Embodiment 59. The interactive visualization system of any one of embodiments 56-58, further comprising: a user input device for receiving a user-selected parameter under which to analyze the immune cell data.

Embodiment 60. The interactive visualization system of any one of embodiments 56-59, wherein the immune cell data comprises a dataset of a B cell receptor, a T cell receptor, an antibody, a single-chain variable fragment (ScFv), an antigen-binding fragment (Fab), or a combination thereof

Embodiment 61. The interactive visualization system of any one of embodiments 56-60, wherein the schema comprises selecting amino acids with a frequency meeting a pre-selected frequency threshold in the clonotype group.

Embodiment 62. The interactive visualization system of any one of embodiments 56-61, wherein the schema comprises selecting amino acids with a selected chemical identity in the clonotype group.

Embodiment 63. The interactive visualization system of any one of embodiments 56-62, wherein the schema comprises selecting amino acids of protein motifs that encode post-translational modification in the clonotype group.

Embodiment 64. The interactive visualization system of any one of embodiments 56-63, wherein the schema comprises displaying positions of the selected amino acids in the clonotype group.

Embodiment 65. The interactive visualization system of any one of embodiments 56-64, wherein the schema comprises displaying positions of the selected amino acids in the clonotype group as in complementarity determining regions (CDR) or framework regions (FWR).

Embodiment 66. The interactive visualization system of any one of embodiments 56-65, wherein the schema comprises highlighting the selected amino acids in the clonotype group.

Embodiment 67. The interactive visualization system of any one of embodiments 56-66, wherein the graphic representation comprises an alignment of one or more amino acid sequences in the clonotype group.

Embodiment 68. The interactive visualization system of any one of embodiments 56-67, wherein the graphic representation comprises a comparison of at least one reference sequence to one or more sequences of peptides in the clonotype group.

Embodiment 69. The interactive visualization system of any one of embodiments 56-68, wherein the graphic representation comprises a phylogenetic tree of the clonotype group.

Embodiment 70. The interactive visualization system of any one of embodiments 56-69, wherein the method further comprises building a phylogenetic tree of the clonotype group according to the schema.

Embodiment 71. A method for producing an immunotherapeutic composition from cells selected from immune cell data, the method comprising: obtaining the immune cell data, wherein the immune cell data comprises a dataset of immune cell receptors, antibodies, or fragments thereof from a sample; identifying a clonotype group in the immune cell data; selecting a schema to visualize selected amino acids in the clonotype group based on positions or chemical identity of the selected amino acids; visualizing the selected amino acids in the clonotype group in a graphic representation according to the schema; selecting a cell of interest from the clonotype group based on a pre-defined criterion using the graphic representation; and producing an immunotherapeutic composition using the cell of interest.

Embodiment 72. The method of embodiment 71, wherein the sample comprises single cells and the immune cell data comprises a single cell dataset obtained from the single cells.

Embodiment 73. The method of embodiment 71, wherein the sample comprises a tissue sample and the immune cell data comprises a spatial dataset obtained from the tissue sample.

Embodiment 74. A computer-program product tangibly embodied in a non-transitory machine-readable storage medium, including instructions configured to cause one or more data processors to perform a method for selecting a cell of interest based on immune cell data, the method comprising: obtaining the immune cell data, wherein the immune cell data comprises a dataset of immune cell receptors, antibodies, or fragments thereof from a sample; identifying a clonotype group in the immune cell data; selecting a schema to visualize selected amino acids in the clonotype group based on positions or chemical identity of the selected amino acids; visualizing the selected amino acids in the clonotype group in a graphic representation according to the schema; and selecting a cell of interest from the clonotype group based on a pre-defined criterion using the graphic representation.

Embodiment 75. The method of embodiment 74, wherein the sample comprises single cells and the immune cell data comprises a single cell dataset obtained from the single cells.

Embodiment 76. The method of embodiment 74, wherein the sample comprises a tissue sample and the immune cell data comprises a spatial dataset obtained from the tissue sample.

Further, an embodiment may include part or all of any one or more of embodiments 1-76

### VIII. Additional Considerations

While the present teachings are described in conjunction with various embodiments, it is not intended that the present teachings be limited to such embodiments. On the contrary, the present teachings encompass various alternatives, modifications, and equivalents, as will be appreciated by those of skill in the art.

In describing various embodiments, the specification may have presented a method and/or process as a particular sequence of steps. However, to the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps set forth in the specification should not be construed as limitations on the claims. In addition, the claims directed to the method and/or process should not be limited to the performance of their steps in the order written, and one skilled in the art can readily appreciate that the sequences may be varied and still remain within the spirit and scope of the various embodiments.

1. A method for selecting a cell of interest based on a single cell dataset, the method comprising:
obtaining a single cell dataset, wherein the single cell dataset comprises a dataset of immune cell receptors, antibodies, or fragments thereof from single cells;
identifying a clonotype group in the single cell dataset;
selecting a schema to visualize selected amino acids in the clonotype group based on positions or chemical identity of the selected amino acids;
visualizing the selected amino acids in the clonotype group in a graphic representation according to the schema; and
selecting a cell of interest from the clonotype group based on a pre-defined criterion using the graphic representation.

2. The method of claim 1, wherein the single cell dataset comprises a dataset of a B cell receptor, a T cell receptor, an antibody, a single-chain variable fragment (ScFv), an antigen-binding fragment (Fab), or a combination thereof

3. The method of claim 1, wherein the schema comprises selecting amino acids with a frequency meeting a pre-selected frequency threshold in the clonotype group.

4. The method of claim 1, wherein the schema comprises selecting amino acids with a selected chemical identity in the clonotype group.

5. The method of claim 1, wherein the schema comprises selecting amino acids of protein motifs that encode post-translational modification in the clonotype group.

6. The method of claim 1, wherein the schema comprises displaying positions of the selected amino acids in the clonotype group.

7. The method of claim 1, wherein the schema comprises displaying positions of the selected amino acids in the clonotype group as in complementarity determining regions (CDR) or framework regions (FWR).

8. The method of claim 1, wherein the schema comprises highlighting selected amino acids in the clonotype group.

9. The method of claim 1, wherein the graphic representation comprises an alignment of one or more amino acid sequences of exact subclonotypes in the clonotype group.

10. The method of claim 1, wherein the graphic representation comprises a comparison of at least one reference sequence to one or more amino acid sequences in the clonotype group.

11. The method of claim 1, wherein the graphic representation comprises a phylogenetic tree of the clonotype group.

12. The method of claim 1, further comprising:
building a phylogenetic tree of the clonotype group according to the schema; and
calculating a distance between each two exact subclonotypes in the clonotype group and between each exact subclonotype and a reference sequence.

13. The method of claim 1, wherein selecting the cell of interest from the clonotype group comprises:
selecting the cell of interest that has a constant region meeting a pre-defined constant region criterion and that has a distance between the cell of interest and a reference sequence at a heavy chain and a light chain level meeting a pre-defined distance criterion.

14. An interactive visualization system comprising:
a data source for obtaining a single cell dataset, wherein the single cell dataset comprises a dataset of immune cell receptors, antibodies, or fragments thereof from single cells;
one or more data processors;
a computing device communicatively connected to the data source and configured to receive the single cell dataset, the computing device comprising a non-transitory computer readable storage medium containing instructions which, when executed on the one or more data processors, cause the one or more data processors to perform a method, the method comprising:
identifying a clonotype group in the single cell dataset;
selecting a schema to visualize selected amino acids in the clonotype group based on positions or chemical identity of the selected amino acids; and
visualizing the selected amino acids in the clonotype group in a graphic representation according to the schema; and
a display for rendering a visualization of the selected amino acids in the clonotype group in the graphic representation according to the schema.

15. The system of claim 14, further comprising:
a user input device for receiving a user-selected parameter under which to analyze the dataset.

16. The system of claim 14, wherein the single cell dataset comprises a dataset of a B cell receptor, a T cell receptor, an antibody, a single-chain variable fragment (ScFv), an antigen-binding fragment (Fab), or a combination thereof.

17. The system of claim 14, wherein the schema comprises selecting amino acids with a frequency meeting a pre-selected frequency threshold in the clonotype group.

18. The system of claim 14, wherein the schema comprises selecting amino acids with a selected chemical identity in the clonotype group.

19. The system of claim 14, wherein the schema comprises selecting amino acids of protein motifs that encode post-translational modification in the clonotype group.

20. The system of claim 14, wherein the schema comprises displaying positions of the selected amino acids in the clonotype group.

21. The system of claim 14, wherein the schema comprises displaying positions of the selected amino acids in the clonotype group as in complementarity determining regions (CDR) or framework regions (FWR).

22. The system of claim 14, wherein the schema comprises highlighting the selected amino acids in the clonotype group.

23. The system of claim 14, wherein the graphic representation comprises an alignment of one or more amino acid sequences in the clonotype group.

24. The system of claim 14, wherein the graphic representation comprises a comparison of at least one reference sequence to one or more sequences of peptides in the clonotype group.

25. The system of claim 14, wherein the graphic representation comprises a phylogenetic tree of the clonotype group.

26. The system of claim 14, wherein the method further comprises building a phylogenetic tree of the clonotype group according to the schema.

27. A method for producing an immunotherapeutic composition from cells selected from a single cell dataset, the method comprising:
obtaining a single cell dataset, wherein the single cell dataset comprises a dataset of immune cell receptors, antibodies, or fragments thereof from single cells;
identifying a clonotype group in the single cell dataset;
selecting a schema to visualize selected amino acids in the clonotype group based on positions or chemical identity of the selected amino acids;
visualizing the selected amino acids in the clonotype group in a graphic representation according to the schema;
selecting a cell of interest from the clonotype group based on a pre-defined criterion using the graphic representation; and
producing an immunotherapeutic composition using the cell of interest.

28. A graphical user interface (GUI) for displaying immune cell clonotyping information, the GUI comprising:
a listing of exact subclonotypes of an immune cell clonotype, wherein the exact subclonotypes share identical V(D)J transcripts;
a listing of one or more textual frames with information about chains common to each member of the immune cell clonotype, wherein a textual frame of the one or more textual frames contains an amino acid sequence for variable and constant regions of each exact subclonotype of the exact subclonotypes; and
positional information for selected amino acids of the amino acid sequence, wherein the selected amino acids are selected based on positions or chemical identity of the selected amino acids.

29. The graphical user interface of claim 28, wherein the listing of the one or more textual frames comprises two or more textual frames. The graphical user interface of claim 28, wherein the listing of the one or more textual frames comprises two textual frames.

31. The graphical user interface of claim 28, wherein the listing of the one or more textual frames comprises three textual frames.

32. The graphical user interface of claim 28, wherein the listing of the one or more textual frames includes a comparison of at least one reference sequence to an exact subclonotype of the exact subclonotypes.

33. The graphical user interface of claim 32, wherein the at least one reference sequence includes a reference sequence listing selected from the group consisting of a universal reference sequence, a donor reference sequence, and combinations thereof.

34. The graphical user interface of claim 28, wherein the listing of the one or more textual frames includes a listing of amino acid alignments of each exact subclonotypes of the immune cell clonotype.

35. The graphical user interface of claim 28, wherein the listing of the exact subclonotypes includes subclonotype information selected from the group consisting of gene expression, Hamming distance, antibody, and combinations thereof.

36. The graphical user interface of claim 35, wherein the gene expression of the subclonotype information is selected from the group consisting of median gene expression, maximum gene expression, mean gene expression, and combinations thereof.

37. The graphical user interface of claim 35, wherein the gene expression of the subclonotype information is reported as a UMI count.

38. The graphical user interface of claim 28, wherein for each exact subclonotype of the exact subclonotypes, the textual frame provides chain-specific subclonotype information selected from the group consisting of V(D)J UMI count, V(D)J read count, constant region name, complementarity-determining region (CDR) sequence, constant sequence length, 5′UTR sequence length, differences from a universal reference constant region, differences from the 5′UTR sequence, base differences between exact subclonotypes, and combinations thereof.

39. The graphical user interface of claim 28, further comprising a user input section to receive information configured to customize a display of immune cell clonotyping information.

40. A computer-program product tangibly embodied in a non-transitory machine-readable storage medium, including instructions configured to cause one or more data processors to perform a method for selecting a cell of interest based on a single cell dataset, the method comprising:
obtaining a single cell dataset, wherein the single cell dataset comprises a dataset of immune cell receptors, antibodies, or fragments thereof from single cells;
identifying a clonotype group in the single cell dataset;
selecting a schema to visualize selected amino acids in the clonotype group based on positions or chemical identity of the selected amino acids;
visualizing the selected amino acids in the clonotype group in a graphic representation according to the schema; and
selecting a cell of interest from the clonotype group based on a pre-defined criterion using the graphic representation.

41. A method for selecting a cell of interest based on immune cell data, the method comprising:
obtaining the immune cell data, wherein the immune cell data comprises a dataset of immune cell receptors, antibodies, or fragments thereof from a sample;
identifying a clonotype group in the immune cell data;
selecting a schema to visualize selected amino acids in the clonotype group based on positions or chemical identity of the selected amino acids;
visualizing the selected amino acids in the clonotype group in a graphic representation according to the schema; and
selecting a cell of interest from the clonotype group based on a pre-defined criterion using the graphic representation.

42. The method of claim 41, wherein the immune cell data comprises a dataset of a B cell receptor, a T cell receptor, an antibody, a single-chain variable fragment (ScFv), an antigen-binding fragment (Fab), or a combination thereof

43. The method of claim 41 or claim 42, wherein the schema comprises selecting amino acids with a frequency meeting a pre-selected frequency threshold in the clonotype group.

44. The method of any one of claims 41-43, wherein the schema comprises selecting amino acids with a selected chemical identity in the clonotype group.

45. The method of any one of claims 41-44, wherein the schema comprises selecting amino acids of protein motifs that encode post-translational modification in the clonotype group.

46. The method of any one of claims 41-45, wherein the schema comprises displaying positions of the selected amino acids in the clonotype group.

47. The method of any one of claims 41-46, wherein the schema comprises displaying positions of the selected amino acids in the clonotype group as in complementarity determining regions (CDR) or framework regions (FWR).

48. The method of any one of claims 41-47, wherein the schema comprises highlighting selected amino acids in the clonotype group.

49. The method of any one of claims 41-48, wherein the graphic representation comprises an alignment of one or more amino acid sequences of exact subclonotypes in the clonotype group.

50. The method of any one of claims 41-49, wherein the graphic representation comprises a comparison of at least one reference sequence to one or more amino acid sequences in the clonotype group.

51. The method of any one of claims 41-50, wherein the graphic representation comprises a phylogenetic tree of the clonotype group.

52. The method of any one of claims 41-51, further comprising:
building a phylogenetic tree of the clonotype group according to the schema; and
calculating a distance between each two exact subclonotypes in the clonotype group and between each exact subclonotype and a reference sequence.

53. The method of any one of claims 41-52, wherein selecting the cell of interest from the clonotype group comprises:
selecting the cell of interest that has a constant region meeting a pre-defined constant region criterion and that has a distance between the cell of interest and a reference sequence at a heavy chain and a light chain level meeting a pre-defined distance criterion.

54. The method of any one of claims 41-53, wherein the sample comprises single cells and the immune cell data comprises a single cell dataset obtained from the single cells.

55. The method of any one of claims 41-53, wherein the sample comprises a tissue sample and the immune cell data comprises a spatial dataset obtained from the tissue sample.

56. An interactive visualization system comprising:
a data source for obtaining immune cell data, wherein the immune cell data comprises a dataset of immune cell receptors, antibodies, or fragments thereof from a sample;
a computing device communicatively connected to the data source and configured to receive the immune cell data, the computing device comprising a set of processors and a non-transitory computer readable storage medium containing instructions which, when executed by the set of processors, cause the set of processors to perform a method comprising:
identifying a clonotype group in the immune cell data;
selecting a schema to visualize selected amino acids in the clonotype group based on positions or chemical identity of the selected amino acids; and
visualizing the selected amino acids in the clonotype group in a graphic representation according to the schema; and
a display for rendering a visualization of the selected amino acids in the clonotype group in the graphic representation according to the schema.

57. The interactive visualization system of claim 56, wherein the sample comprises single cells and the immune cell data comprises a single cell dataset obtained from the single cells.

58. The interactive visualization system of claim 56, wherein the sample comprises a tissue sample and the immune cell data comprises a spatial dataset obtained from the tissue sample.

59. The interactive visualization system of any one of claims 56-58, further comprising:
a user input device for receiving a user-selected parameter under which to analyze the immune cell data.

60. The interactive visualization system of any one of claims 56-59, wherein the immune cell data comprises a dataset of a B cell receptor, a T cell receptor, an antibody, a single-chain variable fragment (ScFv), an antigen-binding fragment (Fab), or a combination thereof.

61. The interactive visualization system of any one of claims 56-60, wherein the schema comprises selecting amino acids with a frequency meeting a pre-selected frequency threshold in the clonotype group.

62. The interactive visualization system of any one of claims 56-61, wherein the schema comprises selecting amino acids with a selected chemical identity in the clonotype group.

63. The interactive visualization system of any one of claims 56-62, wherein the schema comprises selecting amino acids of protein motifs that encode post-translational modification in the clonotype group.

64. The interactive visualization system of any one of claims 56-63, wherein the schema comprises displaying positions of the selected amino acids in the clonotype group.

65. The interactive visualization system of any one of claims 56-64, wherein the schema comprises displaying positions of the selected amino acids in the clonotype group as in complementarity determining regions (CDR) or framework regions (FWR).

66. The interactive visualization system of any one of claims 56-65, wherein the schema comprises highlighting the selected amino acids in the clonotype group.

67. The interactive visualization system of any one of claims 56-66, wherein the graphic representation comprises an alignment of one or more amino acid sequences in the clonotype group.


SEQUENCE LISTING (34 sequences)

SEQ ID NO: 1 - protein, 17 aa, synthetic construct
  REGION 1..17: note = Description of Artificial Sequence: Synthetic peptide
CARVRDILTGDYGMDVW

SEQ ID NO: 2 - protein, 20 aa, synthetic construct
  REGION 1..20: note = Description of Artificial Sequence: Synthetic peptide
HLLSSSQPGYIYSYPIVTLA

SEQ ID NO: 3 - protein, 5 aa, synthetic construct
  REGION 1..5: note = Description of Artificial Sequence: Synthetic peptide
GMDVW

SEQ ID NO: 4 - protein, 20 aa, synthetic construct
  REGION 1..20: note = Description of Artificial Sequence: Synthetic peptide
HLLSSSQPGYIYSYPMVTLA

SEQ ID NO: 5 - protein, 8 aa, synthetic construct
  REGION 1..8: note = Description of Artificial Sequence: Synthetic peptide
PVSQYASQ

SEQ ID NO: 6 - protein, 20 aa, synthetic construct
  REGION 1..20: note = Description of Artificial Sequence: Synthetic peptide
HLLSSSQAGRITSYPMVTLA

SEQ ID NO: 7 - protein, 20 aa, synthetic construct
  REGION 1..20: note = Description of Artificial Sequence: Synthetic peptide
HLLSTNQAGRIASHPMVALA

SEQ ID NO: 8 - protein, 17 aa, synthetic construct
  REGION 1..17: note = Description of Artificial Sequence: Synthetic peptide
CARARDILTGDYGMDVW

SEQ ID NO: 9 - protein, 20 aa, synthetic construct
  REGION 1..20: note = Description of Artificial Sequence: Synthetic peptide
HLLSSNQAGRINRYPMATVA

SEQ ID NO: 10 - protein, 17 aa, synthetic construct
  REGION 1..17: note = Description of Artificial Sequence: Synthetic peptide
CARTRDILTGDYGMDVW

SEQ ID NO: 11 - protein, 12 aa, synthetic construct [sequence omitted]

SEQ ID NO: 12 - protein, 8 aa, synthetic construct
  REGION 1..8: note = Description of Artificial Sequence: Synthetic peptide
PVNQHSSQ

SEQ ID NO: 13 - protein, 12 aa, synthetic construct
  REGION 1..12: note = Description of Artificial Sequence: Synthetic peptide
CQQSFSTLSLTF

SEQ ID NO: 14 - protein, 8 aa, synthetic construct
  REGION 1..8: note = Description of Artificial Sequence: Synthetic peptide
PVNQYARQ

SEQ ID NO: 15 - protein, 6 aa, synthetic construct
  REGION 1..6: note = Description of Artificial Sequence: Synthetic peptide
HLLSSS

SEQ ID NO: 16 - protein, 19 aa, synthetic construct
  REGION 1..19: note = Description of Artificial Sequence: Synthetic peptide
YWSWIRQPPGKGLEWIGYI

SEQ ID NO: 17 - protein, 36 aa, synthetic construct
  REGION 1..36: note = Description of Artificial Sequence: Synthetic polypeptide
TNYNPSLKSRVTISVDTSKNQFSLKLSSVTAADTAV

SEQ ID NO: 18 - protein, 36 aa, synthetic construct
  REGION 1..36: note = Description of Artificial Sequence: Synthetic polypeptide
TNYNPSLKSRVTMSVDTSKNQFSLKLSSVTAADTAV

SEQ ID NO: 19 - protein, 4 aa, synthetic construct
  REGION 1..4: note = Description of Artificial Sequence: Synthetic peptide
WYQQ

SEQ ID NO: 20 - protein, 19 aa, synthetic construct
  REGION 1..19: note = Description of Artificial Sequence: Synthetic peptide
YWSWIRQPAGKGLEWIGRI

SEQ ID NO: 21 - protein, 38 aa, synthetic construct
  REGION 1..38: note = Description of Artificial Sequence: Synthetic polypeptide
TNYNPSLKSRVTMSVDTSKNQFSLKLSSVTAADTAVYY

SEQ ID NO: 22 - protein, 6 aa, synthetic construct
  REGION 1..6: note = Description of Artificial Sequence: Synthetic peptide
HLLSTN

SEQ ID NO: 23 - protein, 38 aa, synthetic construct
  REGION 1..38: note = Description of Artificial Sequence: Synthetic polypeptide
TNHNPSLKSRVTMSVDASKNQFSLKLSSVTAADTAVYY

SEQ ID NO: 24 - protein, 6 aa, synthetic construct
  REGION 1..6: note = Description of Artificial Sequence: Synthetic peptide
HLLSSN

SEQ ID NO: 25 - protein, 38 aa, synthetic construct
  REGION 1..38: note = Description of Artificial Sequence: Synthetic polypeptide
TNYNPSLKSRVTMSADTSKNQFSLKVSSVTAADTAVYY

SEQ ID NO: 26 - protein, 20 aa, synthetic construct
  REGION 1..20: note = Description of Artificial Sequence: Synthetic peptide
CARLGVYYDSSGYHDAFDIW

SEQ ID NO: 27 - protein, 19 aa, synthetic construct
  REGION 1..19: note = Description of Artificial Sequence: Synthetic peptide
WISWVRQMPGKGLEWMGRI

SEQ ID NO: 28 - protein, 6 aa, synthetic construct
  REGION 1..6: note = Description of Artificial Sequence: Synthetic peptide
DPSDSY

SEQ ID NO: 29 - protein, 15 aa, synthetic construct
  REGION 1..15: note = Description of Artificial Sequence: Synthetic peptide
WYQQKPGQAPVLVIY

SEQ ID NO: 30 - protein, 7 aa, synthetic construct
  REGION 1..7: note = Description of Artificial Sequence: Synthetic peptide
GKNNRPS

SEQ ID NO: 31 - protein, 19 aa, synthetic construct
  REGION 1..19: note = Description of Artificial Sequence: Synthetic peptide
WITWVRHMPGKGLEWMGRI

SEQ ID NO: 32 - protein, 6 aa, synthetic construct
  REGION 1..6: note = Description of Artificial Sequence: Synthetic peptide
DPPDSY

SEQ ID NO: 33 - protein, 15 aa, synthetic construct
  REGION 1..15: note = Description of Artificial Sequence: Synthetic peptide
WYQQKPGQAPVLVNY

SEQ ID NO: 34 - protein, 13 aa, synthetic construct [sequence omitted]